(19) United States
(12) Patent Application Publication
(10) Pub. No.: US 2012/0311737 A1
(43) Pub. Date: Dec. 6, 2012

(54) INDUCTION OF APOMIXIS IN SEXUALLY REPRODUCING CULTIVATED PLANTS AND USE FOR PRODUCING TOTALLY OR PARTIALLY APOMICTIC PLANTS

(76) Inventors: Daniel Grimanelli, Assas (FR); Olivier Leblanc, Saint Croix de Quitillargues (FR); Marcelina Garcia Aguilar, Montpellier (FR)

(21) Appl. No.: 13/508,671

(22) PCT Filed: Nov. 9, 2010
(86) PCT No.: PCT/IB2010/055084

§ 371 Date: Aug. 6, 2012

(30) Foreign Application Priority Data
Nov. 9, 2009 (FR) 09/05375

(51) Int. Cl. C12N 15/82; A01H 5/10; A01H 5/00

(52) U.S. Cl. 800/278; 800/320.1; 800/320.2; 800/320.3

(57) ABSTRACT

The invention relates to the use, for producing partially or totally apomictic plants, of a gene, of a transcript of this gene, or of the ORF thereof, encoding a protein comprising a DNA methyltransferase motif.

The invention relates to means for regulating reproductive development in cultivated plants. More particularly, the invention relates to the development of plants reproducing totally or partially by gametophytic apomixis, i.e. asexually by means of seeds.

Gametophytic apomixis is a form of asexual reproduction by seeds. It occurs in many angiosperms, and nearly 400 apomictic species have been recorded. However, no apomictic plants are found among the principal cultivated cereals (maize, wheat, or rice), but only among wild plants, a few cultivated fodder species, and certain fruit species. Apomixis is a genetically controlled mechanism. Apomictic plants develop female gametes without prior meiosis. The gametes thus formed contain a genome identical to that of the somatic tissues from which they are derived. The embryo develops from these gametes without fertilization by a male gamete, i.e. by parthenogenesis. The genome of the embryo thus formed is therefore strictly identical to that of its mother plant, without any contribution from a father plant. Apomixis is therefore a seed-based means of cloning that ensures identical perpetuation of the genotypes through the generations.

The use of apomixis in a controlled manner in cultivated species offers numerous potential applications. These applications relate to propagation of unstable genotypes, control of pollen contamination, methods of improving plants, and methods of commercial seed production.

None of these applications is conceivable for the main cultivated species such as wheat, maize, rice, and others on the basis of current technologies. In fact, no apomictic forms are known among these various species, and no genetic system is known that would permit the induction of apomixis in sexual plants.

Many laboratories have endeavoured, over the years, to develop apomictic plants, either by trying to transfer the determinants of apomixis from wild plants to cultivated plants, or by inducing, by mutagenesis, apomictic phenotypes in sexual plants. Neither of these two approaches has produced an apomictic genotype in a species where this mode of reproduction did not exist before.

Recent results, published recently in the journal Nature (Ravi, M., Marimuthu, M. P., and Siddiqi, I. (2008). Gamete formation without meiosis in Arabidopsis. Nature 451: 1121-1124), show in Arabidopsis that the inactivation of a gene involved in meiosis, called DYAD, whose function is to regulate the cohesion of the chromatids during meiosis, makes it possible to produce about 0.1% of gametes that bypass meiosis (Ravi et al., Nature 2008-451:1121-1124). The rest of the gametes, and therefore 99.9% of the offspring, are sterile. It is even probable that the frequency of nonmeiotic gametes is not significantly different in these mutant plants from that in sexual plants, and that these gametes in fact only appear in the observation because, moreover, the mutation kills all the gametes normally resulting from sexuality. Another recent work (by Erfurth, I., Jolivet, S., Froger, N., Catrice, O., Novatchkova, M., and Mercier, R. (2009). Turning meiosis into mitosis. PLoS Biol. 7: e1000124.), which is much more promising, shows that it is possible, in Arabidopsis, to change meiotic division into mitotic division by simultaneous inactivation of three genes implicated in meiosis (osd1/Atspo11-1/Atrec8). The triple mutant produces functional diploid gametes. These gametes are, however, fertilized, therefore the descendants are not apomictic, and it is not known whether this result is transferable to species other than Arabidopsis.

The inventors' work in this field has shown that it is possible to induce a phenotype that is totally or partially apomictic in maize by manipulating the expression of several genes that are collectively involved in the regulation of gene expression in the female reproductive organs (ovules) of maize. The seeds produced do not go through meiotic reduction and are fertile. These results apply advantageously to other cultivated plants such as rice or wheat.

The invention therefore relates to the use of specific nucleotide sequences whose manipulation permits the development of plants reproducing totally or partially by gametophytic apomixis.

It also has the aim of providing a method of producing apomictic plants.

According to yet another aspect, the invention aims to use apomixis in a controlled manner in sexually reproducing cultivated species for developing a great many applications, as will be described below.

The invention thus relates, for the production of partially or totally apomictic plants, to the use of a gene coding for a protein with a DNA methyltransferase motif. It is more particularly a gene of the family of DNA methyltransferases coding for a protein of sequence SEQ ID No.1 or SEQ ID No.5.

According to one embodiment, the invention relates to the use of the DMT103 gene of the family of DNA methyltransferases corresponding to sequence SEQ ID No.2 or of the transcript of such a gene corresponding to sequence SEQ ID No.3, or of the ORF of sequence SEQ ID No.4.

In another embodiment, the invention relates to the use of the DMT102 gene of the family of DNA methyltransferases, corresponding to sequence SEQ ID No.6, or of the transcript of such a gene corresponding to sequence SEQ ID No.7, or of the ORF of sequence SEQ ID No.8.

These genes are expressed specifically in the ovules where the reproductive cells are determined.

The inactivation of these genes by mutagenesis and therefore of the transcripts and of the proteins in sexual plants leads to the formation of unreduced gametes and of multiple gametophytes in the ovule, which are characteristic of apomictic development.

The invention also relates to a method for inducing, in cultivated species such as maize, rice or wheat, a totally or partially apomictic phenotype, characterized in that it comprises the targeted inactivation, by a transposable element, for example of the Mutator type, of a gene, of a gene transcript or of its ORF, such as are defined above, and identification of the mutated locus.

The use of apomixis in a controlled manner in cultivated species offers numerous potential applications. These include propagation of unstable genotypes, control of pollen contaminations, methods of improving plants, and methods of commercial seed production.

The first application relates to clonal propagation, by seed, of genetically unstable genotypes. This is the case in particular for all hybrid plants; these hybrid plants produce, by genetic mixing during meiosis and fertilization, descendants that are different from one another, and different from their mother plant. It is also the case with cultivated species displaying unstable levels of ploidy in meiosis, such as triploid forms.

In the majority of cultivated species it is necessary, in order to maintain a high level of genetic purity, for pollination to be rigorously controlled, to avoid contamination by pollen from neighbouring fields, at varying distances, as the pollen can move over quite variable distances, depending on the species, the climatic conditions, or dissemination vectors such as insects. In the case of apomictic plants, however, the genome that comes from the male gametes does not participate in the next generation. The use of apomictic plants would therefore make it possible to avoid the risks of contamination. Apomixis therefore constitutes a completely unique method of controlling genetic purity. It is also potentially an effective method of avoiding undesirable flows of transgenes when growing genetically modified organisms.

Apomixis also offers new perspectives in plant improvement. It would in fact make it possible to use, as a new variety, any genotype selected as interesting, since it is a genetically determined criterion, regardless of its genetic structure, since the latter, as it is apomictic, becomes genetically stable. We can therefore envisage developing varieties directly from hybrid forms, optionally interspecific, avoiding the stabilization steps currently required, such as successive steps of self-fertilization, or the production of doubled haploids. This method therefore gives a considerable time saving, but also certainly opens the door to the introduction of completely new genetic materials in selection programs, and in particular of genetic materials which, in sexual plants, induce pronounced sterility. This is the case for example with most interspecific crosses.

A very important application relates to the production of hybrid seeds. As practiced today, the latter involves the large-scale controlled hybridization of genetically stable parental ecotypes. They are generally homozygotic lines, obtained by various methods (production of doubled haploids, self-fertilization, etc.). One of the two parents is used as the male, the other as the female. Only the females produce commercial seeds. The yield of the seed production plots is generally low compared with the hybrids, for three reasons: (1) the male lines are necessary but use a high proportion of the space without producing seeds; (2) the parental lines generally have a much lower yield than the hybrids as a result of depression of consanguinity; (3) the control of pollinations involves the physical or genetic castration of the lines used as female, a process that leads to a considerable loss of yield. In the case of apomictic plants, however, we could envisage producing seeds directly from hybrids, therefore with much higher yields, using 100% of the available area, without the need to control pollination, and without a castration step. The benefit of using apomixis for producing seed is very significant in species such as maize, where hybrid forms are already produced for reasons of lower costs, but also in autogamous species, for example wheat or rice, where large-scale controlled hybridizations are difficult. The production of a few apomictic hybrid plants would be sufficient for initiating the large-scale production of genetically stable hybrid seeds.

The plants or seeds of partially or totally apomictic plants of cultivated species such as maize, rice and wheat, characterized in that they comprise inactivated alleles of a gene, as defined above, also fall within the scope of the invention.

The plants or seeds of plants of the invention are advantageously those that are obtained by inactivation of the gene by mutagenesis or according to the method as defined above, for inducing a totally or partially apomictic phenotype in cultivated plants.

These plants and these seeds, whose protein with DNA methyltransferase motif is inactivated, produce unreduced gametes and multiple embryo sacs.

Other features and advantages of the invention are given in the following examples for purposes of illustration.

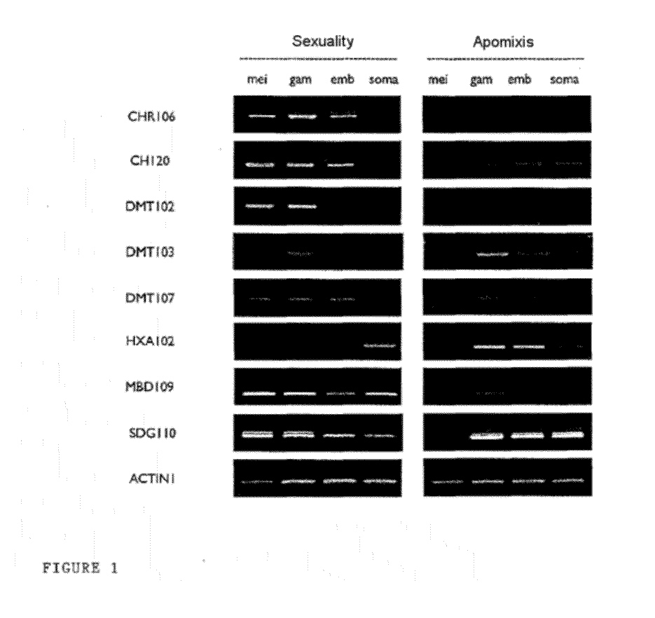

These examples refer to FIGS. 1 to 6, which show, respectively,

FIG. 1: comparison of the expression profiles of the chromatid regulators;

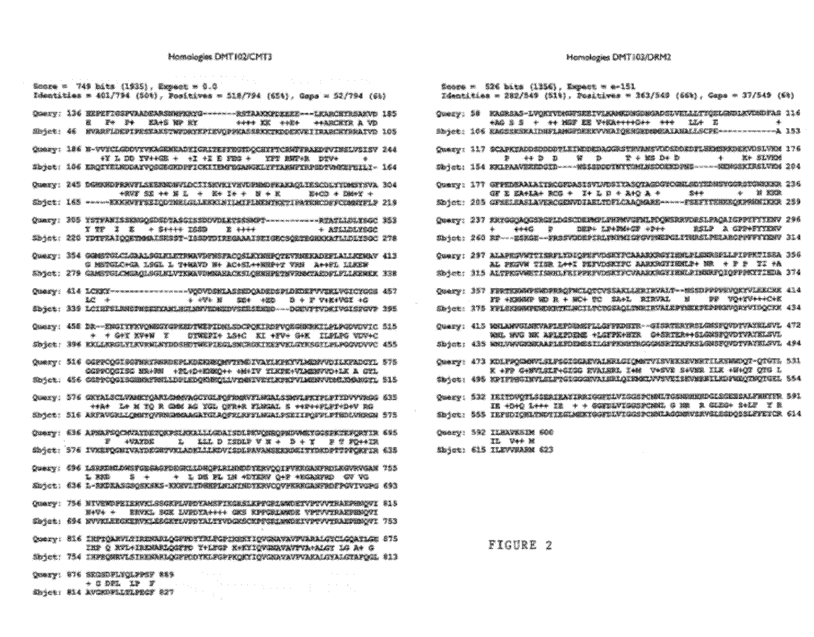

FIG. 2: comparison of protein sequences with the alignment of the protein sequences of DMT102 and CMT3, and DMT103 and DRM2;

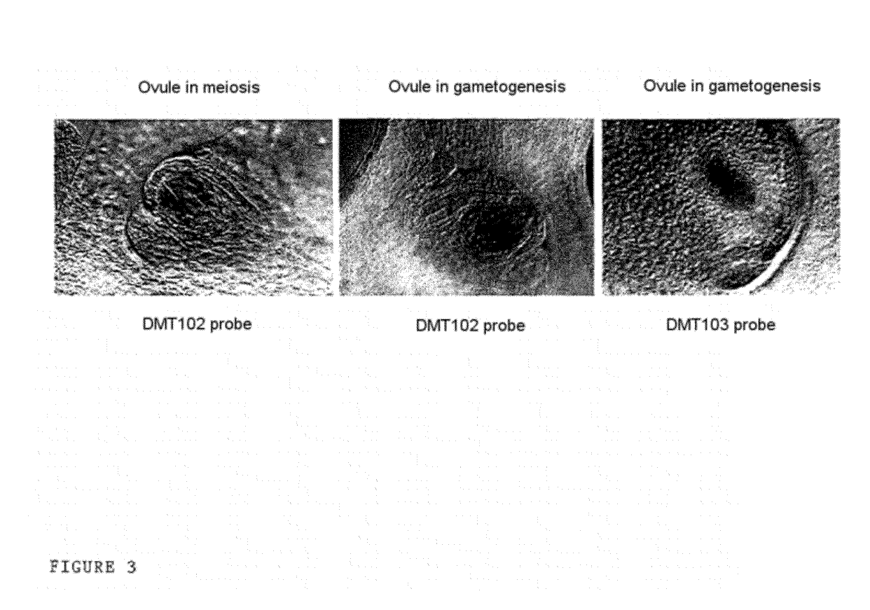

FIG. 3: the expression profile of the two genes by mRNA in situ;

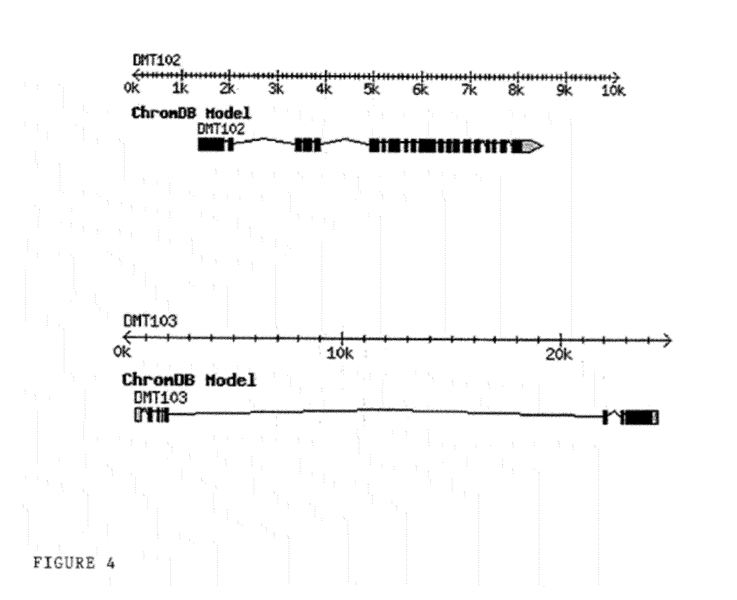

FIG. 4: the structure of the two genes DMT102 and DMT103;

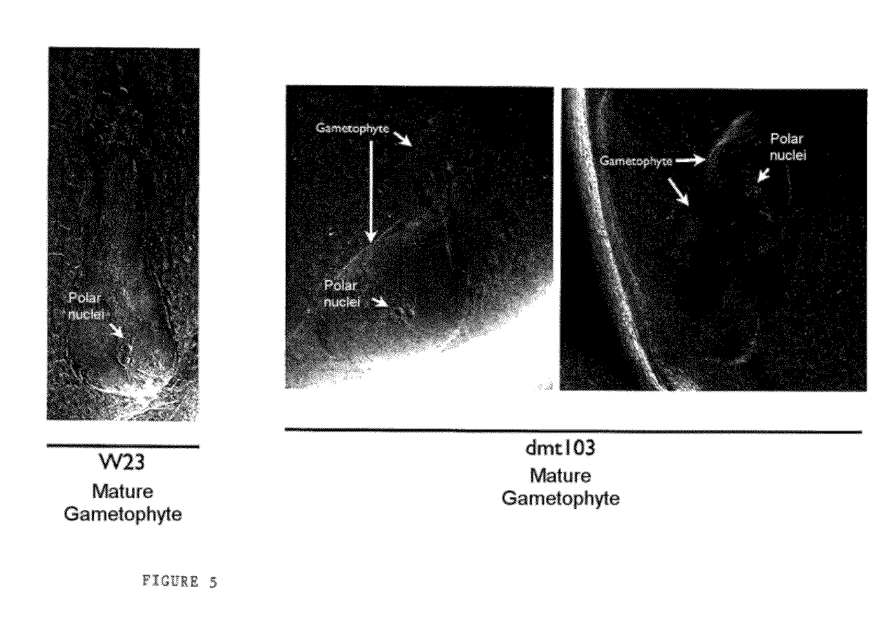

FIG. 5: phenotypes of the mutant plants with production of multiple embryo sacs; and

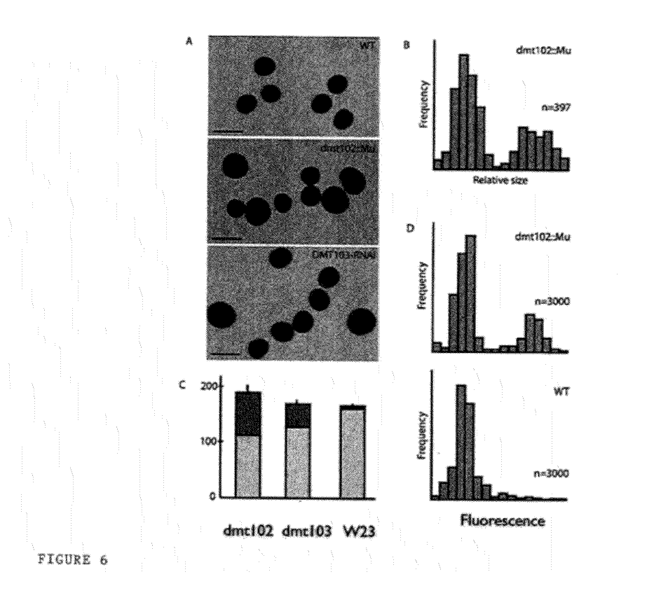

FIG. 6: phenotypes of the mutant plants with production of unreduced gametes.

The sequences SEQ ID No.9, 10, 11 and 12 correspond to those of mutants ago 104-752, 770, 775 and 1352. The sequences SEQ ID No.13 and 14 correspond to those of mutants of DMT103-1042 and DMT103-1342.

### DEFINITIONS

“Gametophyte” is the haploid structure that develops from the products of meiosis, and when mature contains the gametes. It is the embryo sac on the female side, and the pollen grain on the male side. In maize, the ovule only contains one embryo sac.

“Gametophytic apomixis” refers to a form of asexual reproduction by seeds in which the gametes produced in the female gametophytes have not undergone meiotic reduction, and therefore have the same ploidy and the same genetic constitution as the mother plant. Gametophytic apomixis involves two successive steps: apomeiosis and parthenogenesis.

“Apomeiosis” corresponds to the mechanisms by which apomictic plants bypass meiosis.

“Non-reduction” corresponds to gamete formation in the absence of meiotic reduction.

“Unreduced” gametes are therefore gametes which develop in the absence of meiosis, or via a non-reductional meiosis. Apomeiosis is therefore the specific form of non-reduction that we observe in apomictic seedlings.

“Diplospory” is a specific form of gametophytic apomixis, in which the apomeiotic gametes develop from the same cells as those that participate in sexual reproductive development, i.e. the archesporium.

“Apospory” is a specific form of gametophytic apomixis, in which the female gametophytes and the apomeiotic gametes develop from the somatic cells of the ovule. In aposporic plants, sexuality and apomixis coexist functionally, and therefore typically several embryo sacs are found in one and the same ovule, derived either from sexuality or from apospory.

“Parthenogenesis” corresponds to the development of the embryos without fertilization and without a paternal genetic contribution.

### EXAMPLE 1

### Identification of a Regulator of the Structure of Chromatin Deregulated in Apomictic Plants

The experiment described below relates to comparison of the expression profile of genes involved in determination of the structure of chromatin between sexual plants and apomictic plants.

The RNAs of samples corresponding to ovules of apomictic and sexual plants at the following stages of development were isolated: ovules containing a mother cell of the megaspore, ovules containing a functional megaspore, and ovules at the moment of fertilization.

The sexual plants used are two lines of maize with reference B73 and W23.

The apomictic plants are hybrid forms obtained by crossing an apomictic plant of the species Tripsacum dactyloides, a wild relative of maize in which apomictic forms are found, with a sexual maize; these plants were then backcrossed several times to maize, selecting the apomictic descendants, until plants were obtained containing a genome of diploid maize, and a genome of haploid Tripsacum dactyloides.

These plants have already been described in the literature (Grimanelli et al., Genetics, 2003, November, 163(3); 1521-31) and reproduce by apomixis with very strong penetrance of the character, close to 95%. The relatively large size of the reproductive organs in maize permits fine dissection of the tissues used, using a stereomicroscope. A random sample of the tissues sampled is used after each sampling to verify the stages of development.

In this experiment, the expression profile of 386 maize genes belonging to different families of genes that are known to affect the structure of chromatin was determined precisely. These genes were identified using the CHROMDB database, which lists all the genes belonging to these families in the various species whose genome is fully or partially sequenced.

The analysis was carried out in two steps, firstly with selection of the genes that are expressed specifically in the reproductive tissues; then, for these selected genes, analysis of their expression profile at the different stages mentioned above for the apomictic and sexual forms.

Out of 386 genes analyzed, 8 have a profile that is clearly altered.

These results are illustrated in FIG. 1: Legend: mei: ovule in meiosis (sexuality) or apomeiosis (apomixis); gam: ovules in gametogenesis; emb: start of embryogenesis, corresponding to the moment of fertilization; soma: somatic tissues of the plant.

Alteration of the expression profiles may imply complete absence of expression in one form of reproduction relative to another, which is the case for example for DMT102 and CHR 106, expression of which is cancelled completely in apomictic plants.

However, most of the examples illustrate deregulations that are more specific over time. This is the case with CHR 120, DMT103, DMT107, HXA 102, MBD 109 or SGD 110, expression of which is only cancelled at this specific stage of development.

### EXAMPLE 2

### Biological Function of the Genes, Identified by Sequence Homology

The search for homologies for these various genes, and in particular the use of BLAST for comparing them with public databases, makes it possible to ascribe a biological function. It is clear, on the basis of these homologies, that all of the genes identified are involved directly or indirectly in the silencing pathways, and in particular the establishment or maintenance of DNA methylation:

CHR 106 is a homolog in maize of DDM1 in Arabidopsis, an enzyme involved in maintenance of DNA methylation.

DMT102, DMT103 and DMT107 are the homologs in maize of CMT3, and DRM2 or DRM1 respectively in Arabidopsis. DRM1 and 2 and CMT3 act in a partially redundant manner in the control of asymmetric methylation (at the CHH or CHG site).

MBD 109 is a protein with a methyl binding domain, of unknown function, but therefore probably acts on methylated sites.

CHR 120 is a homolog in maize of MOM1 in Arabidopsis, a gene involved in silencing mechanisms.

HXA102 is a homolog in maize of AtADA2 in Arabidopsis, a component of the complex histone acetyltransferase ADA.

SDG110 is a histone methyltransferase, of unknown function.

### EXAMPLE 3

### Expression Profile of the DMT102 and DMT103 Genes in Sexual Maize

The results relating to DMT102 and DMT103 are presented below. They correspond to a pathway that is extremely well characterized in Arabidopsis, called the RdDM (RNA-dependent DNA methylation) pathway.

DMT102 and DMT103 are respectively the homologs of CMT3 (CHROMOMETHYLASE 3) and DRM1 and 2 (DOMAIN REARRANGED METHYLTRANSFERASE 1 and 2) in Arabidopsis. The tissue expression profiles of these two genes (DMT102 and DMT103) were analyzed by hybridization in situ of RNA probes in a sexual maize.

The results are illustrated in FIG. 3. The hybridization profiles obtained confirm the great specificity of expression detected by RT-PCR: the two genes are expressed very specifically during targeted stages of reproductive development. Thus, a signal is detectable for DMT102 immediately before, and then during meiosis. DMT103 is only detected during gametogenesis. The in-situ data show, moreover, that at the tissue level, these two genes are only expressed in a very limited number of cells, corresponding for each ovule, on the one hand to the reproductive cell (the archesporium, the mother cell of the megaspore and the meiocytes during sporogenesis; the gametophyte during gametogenesis), and on the other hand to a small number of cells surrounding the reproductive cell.

The action of these genes is therefore limited by an extremely targeted expression profile from the spatial and temporal standpoint.

This profile points to a large difference between the members of the RdDM pathway identified here and the RdDM pathway as described in Arabidopsis: in this species, the RdDM pathway is essential in the somatic tissues of the plants, where they play a role in maintaining the methylation profiles of the repeat sequences. Their reproductive role in Arabidopsis is unknown, and mutations in the corresponding genes do not have a particular reproductive phenotype, or notable effects on the fertility of the plants. The genes identified here, in contrast, possess an essentially reproductive expression profile.

### EXAMPLE 4

### Phenotype of Mutant Plants for which the Function of DMT102 and DMT103 is Cancelled

To demonstrate the potential role of DMT102 and DMT103 in the expression of apomixis, experiments were conducted for verifying whether inactivation of their function in a sexual plant leads to the same phenotype as that of apomictic plants. It is a matter of verifying that by manipulating the expression of these genes in such a way that their expression is similar to how it is in an apomictic plant, an equivalent phenotypic response is indeed recovered.

Maize plants mutated specifically in these two genes were therefore analyzed, the corresponding mutations cancelling their function. For DMT102, the mutant line corresponds to insertion of a transposon of the Mutator family in the methyltransferase domain of the protein. For DMT103, the mutation arises from the substitution of several amino acids in essential sites: R-49-I, R-272-Q, C-182-Y (residue in the wild form-position-residue in the mutant).

These plants were analyzed for the expression of two characteristics intrinsic to apomictic plants: apomeiosis, and therefore the capacity to produce unreduced gametes, and apospory, and therefore the capacity to produce several embryo sacs in the ovules, but with a single archesporium.

As shown in FIG. 5, the mutant forms of DMT103 produce multiple embryo sacs in a single ovule.

This phenotype has never been described in the literature outside of apomictic plants.

FIG. 6 gives the phenotypes of the mutant plants with production of unreduced gametes: A) the size of the male gametophytes is highly correlated in the plants with the level of ploidy of the gametes that they contain. A plant's capacity to produce unreduced gametes can quickly be assessed by observation of the mature gametophytes, in this case the pollen grains, under a microscope.

WT corresponds to a wild-type plant, the line W23.

dmt102-mu and dmt103 are two mutant forms of DMT102 and DMT103, respectively, and clearly produce gametophytes of variable sizes, as illustrated by the frequency graph B). The frequency of these gametes in the wild-type plants W23, and the two mutant forms is quantified in C). The relation between seed size and the level of ploidy is demonstrated by a flow cytometry analysis (D).

This figure shows that both genes, in their respective mutant forms, produce a high proportion of unreduced gametes, from 30% in DMT103 to 50% in DMT102. These forms are therefore highly apomeiotic.

1. The use, for producing partially or totally apomictic plants, of a gene, of a transcript of this gene, or of its ORF, coding for a protein with a DNA methyltransferase motif.

2. The use as claimed in claim 1, of a gene coding for a protein of sequence SEQ ID No.1 or SEQ ID No.5.

3. The use as claimed in claim 2, characterized in that it is the DMT103 gene corresponding to sequence SEQ ID No.2, or of a transcript of said gene corresponding to sequence SEQ ID No.3, or of its ORF of sequence SEQ ID No.4.

4. The use as claimed in claim 2, characterized in that it is the DMT102 gene corresponding to sequence SEQ ID No.6, or of the transcript of said gene corresponding to sequence SEQ ID No.7, or of its ORF of sequence SEQ ID No.8.

5. The use as claimed in claim 1, characterized in that the gene is inactivated by mutagenesis.

6. A method for inducing, in cultivated species such as maize, rice or wheat, a totally or partially apomictic phenotype, characterized in that it comprises the targeted inactivation, by a transposable element, for example of the Mutator type, of a gene as defined in claim 1, of a transcript of this gene as claimed in claim 1, or of its ORF as claimed in claim 1, and identification of the mutated locus.

7. An application of the method as claimed in claim 6, for the propagation of unstable genotypes, control of pollen contaminations, improvement of plants and commercial seed production.

8. Totally or partially apomictic plants of cultivated species such as maize, rice or wheat, or seeds thereof, characterized in that they comprise inactivated alleles of a gene as defined in claim 1.

9. Totally or partially apomictic plants of cultivated species such as maize, rice or wheat, or seeds thereof, obtained as claimed in claim 5.


SEQUENCE LISTING (14 sequences)

SEQ ID NO: 1 - protein, 603 aa, Artificial sequence
  Methyltransferases
MVHWVSDSDGSDNFEWDSDGNGEQTVSFNAAGAGSSALAATNTDAPGPSTRVANGNGKAGRSASLVQKYVDMGFSEEIVLKAMKDNGDNGADSLVELLLTYQELGNDLKVDNDFASSCAPKTADDSDDDDTLEIWDDEDAGGRSTRVANSVDDSDDEDFLHEMSRKDEKVDSLVKMGFPEDEAALAITRCGPDASISVLVDSIYASQTAGDGYCGNLSDYEDNSYGGRSTGNKKKRKRYGGQAQGSRGPLDGSCDEPMPLPHPMVGFNLPDQWSRRVDRSLPAQAIGPPYFYYENVALAPKGVWTTISRFLYDIQPEFVDSKYFCAAARKRGYIHNLPLENRSPLLPIPPKTISEAFPRTKRWWPSWDPRRQFNCLQTCVSSAKLLERIRVALTNSSDPPPPRVQKYVLEECRKWNLAWVGLNKVAPLEPDEMEFLLGFPKDHTRGISRTERYRSLGNSFQVDTVAYHLSVLKDLFPQGMNVLSLFSGIGGAEVALHRLGIQMNTVISVEKSEVNRTILKSWWDQTQTGTLIEITDVQTLSSERIEAYIRRIGGFDLVIGGSPCNNLTGSNRHHRDGLEGEHSALFHHYFRILHAVKSIMERL

SEQ ID NO: 2 - DNA, 25017 bp, Artificial sequence
  Methyltransferases
ccaacaaactgactgatactcctttcgtccacaaatctctgtcgcccgatcttttttcatttttctattagtcttcgttgtattagcatgttcctctgtcttctcagtatgtctgtcgcttcattacttctgcgcattctttaagaatctttattgatatcgggtattatgaaaacttgtagtgattaaaagcaaatcgacgctccttggtcactgcaccaatgcctaatacgctggagatttaggaatggatggagtataatatacttagaaattaattatataaacatagttaaaaattaatatatatgaagagtcgtgtaaagataaacttttaaatatagttgagagtcaatatatttgagaagaccatcttttatcttttttatttacattaaatgatattgtacctgctctgactaaagcggtcagacggtggagacagacagaggacgactatcttttatatttttatttacattaaatgatattgtatctttattgtcttgccctaaagcggtcagtgggtggaggcagccaagagtcaccctgtcgccgttgccgtcgccgaggcaggcagagtctccctgtcgccgttgccgtcgccggcctcctcctcctctgccacttcctcccaactcccagccgcaggggccgacggcgacgggagggaagaggcggcagaggtactccgaggggcgcggagagaggctgtgccaggccacgccgtccagtgcctccgggtccgtcgccgcagcttccggccgcgtcggagaggtagcccccggagctcttcgcggaggctcgggaatggtggtgagagtttcccttcgcgtcccctcgtcttctctgctctgcctccttccgtgtactgtgaagagatgctatctcgcgcaatgaattcttgacgctaataatatggtccgctgctacggatattccgcgcgttagattggggtctttgatgtggcgaggtcattacggtgtcctagccctcatattcagcgctcagtgttggggttttcttaacactggattgagctagagcttgcgttttggatgtttttttcgagattgatggtggtttggtcctgttgttgatcgatgtggctgatatgtacttcttaattcgtgtcacagcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggtaagccagctaacggatccatacatatatgcaacaatgtgtgagttcagcagctagttcgtgttactcaggggtgttcaatttaaaccacttgtgccaaactggccggggtggggttgggtggtggatataataatcttgaacaaacgcaggatcatgattagtctacagtgacacggacaccctcaatgttactgttaccttgtttctagtggctacgattgtaaggtttctatttgtttgtggggttggttttaaggttgctaatggcaatgggaaggctgggcgatctgcctctttggttcagaagtatgtggacatgggtttctcagaagagattgttctgaaggccatgaaggacaatggtacttaactaatagctcatctcttctcatttatttagattctatgtggtttaatataccctatatcacagtttttttttctttcaggggataatggagcagattcattagttgagctccttcttacttaccaggtattcatttgtgttcctgcttctatgttattctttgatgatgttgttgcatggtatcaaagagaatttattttacaaagtgaataagtgatgccaaatgcaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggtattttcatgctccccttggtgattgtaagcgtttactttattcttttttgagaaacctactatgcatttactttgcttcaatcatatatacaacgacaaacagcaacaacaaaatcttttattccaaacaagttgcgtagactagagttaaaaaattaatagaacaacaagtcatggttcggccacatggatagttgttttttatgaactcatattcaaggctaaatctttgggttgggtatatcccatccttatctccatttactgtctcttcccatgtcaactttagtcttcctcttcctcccttctcaattctcataactaaggcgccttagaattccactacgcacttgcgtctatggaggtctacgttggacatgtccaaaccatctcaaacgttgttggacaagtttttcttcaactggtgctactcctagcctatcacgtatatcatcattccggatttgatctcttcttgtatggccacaaattaaacacaacatacacattttcgggacacttaattgttgaacatgtcgtcttttgtagggcaacattctgcaccatacaacatagaaggtttaatcattgtcctataaatcttttcttttagcttctgtggtaccctcttgtcatatagaactccagatgcttgacgccacttcatccatccgcttcgattctatggctaacatcttcattaatatccccgtctctctatagcattgatcctaaatacctaaatgtgtccttctaggcactgcctgactttccaaactaacatctcctttctcatgtgtagtcccgaaatcacatttcatatattttgttttagttgtactgagtctaaaacctttggattctagagtctctcgccacaactctagtttcctatttactcttgcccaccacataacattccttggtatcttgtcataattttccatgtcaatgaaatgtcacacccggatttaagggataaagccgggtgtgtctcatatgtgcgccaaaaaagaacaacacatataatgataaagtgtatagagataaatgtcacaatatcataaaagtaattattacatagcggaagtcttacaaaaataaatgataaatataaaacgaactaatgtccatccatggcgccataacgttgactgggagacgccacctagttcaactcgtatgcttcattgtagggcggctcctcttcgaccacctgctattaacctgtggggggggggggggtttatatatcgcaagagtgagctcacaaaatatcatagcttaagtgattaagtgagaactcaagaagaaaaatcaatcacaataaaagatgacacaagagacacagtgattttatcccgtggttcagccaatgcctactccacgttgtggtgacctccttcggtcaaggattgcactcaatccctctcaagtgatccaacaatcaaacttgagtatcacagttttcttccttagtgatgtttcccgtttgtgaggaatctccacaagttggagcctctcacccttacaatgttgatcacaagaaaagcacaagagtaagggagggaaagaaacacacacaagactagaatcgcaacaacaatacacacacaagccaagaagagagcacacaaaaacaatgccacaagttcacaacacacttagtgctcaaatctcagtcacgatgatccaaatgcgtatttgcggagtctaggcgtcttaggatgtttagagaatgcttggtgtagtgctccatgcgcctaggggttccttttatagccccaaggcagctaggagccgttggaattccatttggaaaggtaattcttgcctttagaagcttatgaaggttcttcattccaacatgtgctagatggcggtgccagagccagcccatattagtcttagcgattaagcatgcatctagatcgacattctcttttgagaaatcaactaaatagagcttgccgtgtaatacagccttaaaagttaatgaacaatcactccttctaaagacagatacatcaatatttgtaaataaacaattatagcccatatgacataattgacttacaaacaataaattatacccgagcgattctactaaaaacacatttgaaattgaatgctcggttgtgatggctatcttgcttaagcccttaaccttgccttggtttccatctccaaaaatgatagtatcttgggaatccctgttcttgatgtaagaggtgaacattttcttctcccccgtcatgtggtttttgcatccgctatcaataatccagcttgagctcccggatgcataaacctgcaaggtaattaagcttggcttttaggtacccaagtcttgttgggtcctacaaggttagttacaacaaattttggaacccaaatgcatgtttctcccttgcatttagatcccaattttctagcaactactttggcatttttacatgacaatacaaaagaaacattacatgtttgatacatagtagttggaccagtgtatactttcctaggtgcatgagaatcactatgattacgcctaggcctacttctaccatgaacatatgtggagctagatgcaaacatagcatgagaattatgataagtaggtagagcaacatgattataacacttatcatgatgggcaacactggaaattttcttgacatgagcataaagataagcatgattcttttgaccatgtgaagaatacaagctactagccataggagccttccctttctctttgttgagactggaagtcctttggcttgttaagttcttggtttcttatcggaaaccaagtccatccttaattgaggggtgtctaccaatgatgtaggcatccctagcaaatttgagtttctcataattatctttgcaagtcttaagttgagcattaagacttgcaacatcattgtttaacttaataatagtagaattatgttcattacaagcatcaacgttaaaatttttgcatctattgcaaataacaacatgctctatggatgaactagcagaatttgcttcttttaacttagcatttaaatcatcatttaaattctttaggctagagatagattcattataagcagataattcagaagtcaatagttcattcttcttagtttctagagcaagggctttttgaacattaatgaatttatcatgttcttcataaagaatatcttcttgcttttctagaagcctatccttttcattaagagcatcaattaattcatttattttgtctactttagttttatctaatcctttaaacaaatcagtatagtccacttcatcatcagatgattcctcatcactagaagaagaatacttgggggtatctcgaatacgtacctttttctccttagccatgaggcacgtgtgtcgttcgttggagaagaggaaagacttgttgaaggtcgaggctagtccttcatcatcgaagtcggatgaagaacagtcggagtcccactctttttcaatgtgcgcctcgcctttcgccttcctgtagttcttctttctttccttcttcccatgcttctcttgtgcctggtcattatcgttatcgaggcgttgtgctataaaatgaccagacttaccgcatttgaagcaggagcattttccccttgatttgctcttgttagggtactccttgcgtccttttaaggcggtcttgaagcgcttgatgatgagtgccatctcatcctcgttgagcccggcatcctccacttgtgctagcttgctaggtagtacctccttgctgctggttgctttgagagcaacgggctgcgattcgtgtagaggaagcggtccgtttaatgcatcatccacatacctcgcttcctttaccatcatgcgcccgctaacgaattttccgatgatttcctcgggcgtcatcttggtgtacctggggttctcacgattaaggtttataacatgaggatcaataacagtaaatgacctgagcatgagtcggacgacgtcatgatccgtccatcttgtactcccgtagcttcggattttgttgacaagggtcttgagcctgttgtacgtttgtgttggctcttctccccttatcatcgcgaatctccctagctcgccttccaccagttccattttagggattatggtggcatcatttccctcgtgagaattttgagggtgtctcatatttgtttggcattatctaggccactttgttgtattcatccctgcatagagatgctaataaaatagtagtagcttgtgtatttttgtgtatttgctcattgataaacacagggttgttagagctatcaaaatgcattcaattttcaacaatctcccagatactaggatggagggagaataaatgactacgcattttatgactccaaaaagaataatcctctccatcaaagtgtgggggttttccaagtggaatagagagcaaatgggcatttgaattgtaggggatgcgagaataatcaaaggaataattttgattaagcgttttctttttcgatgaagagtcttcgtcgtcgtcatcccttggtgaagaggaggcgtcactgtcgtagtagatgatcttctttatgtgcctcttcttctttctgtccttcttcttgtgacttgagcctgagtcagtgggcttgtcatctcttggctcgttgaagagggactccttctccttgtcgttgatcaccatccccttacccttaggatccatctctttggtcggttagtccctaaatgaagagtacgactctgctaccaattgagagcacctagaggggggtgaataggtgatcctgtaaaatcaagtactaaatagtccaaacttggttacgaaatgttagttagatcaaaaccaagatgctatagcgagaactcttcacttgattgttcctttagaatgagtattaaacttaggaacaagagcacaagtgattaagcggcctatgatggtttctttattggctcaacaacctttaggtcttggaggaggatttggaagagttgggctcctggaaaatgtaaattcttcatgtggctggtggcgcacggccgctgctggaccgctgaccggctggcaaggagaggcctgcctcatcctcaaagttgtctgttatgtgaccaggaggaagaaactatgaatcatctgctagtttcctgtgtcttctcaagggaagtctggtttaatatcctgaaaacggtcggtttgcagcacctctctcccagtctcgattcttcttcttttgatgattggtgggagaatgtaagcatgacccttattagcccctccgataagaagctgcgcaaggggctgaactcccttatcattttaggagcttgggccatttggattcatcgcaatcaatgtgtcttcaacggagagcagcctagggctagcaaggtcataaattgggtcatggctgagtctcatctctggtgtagggctggggctcgcggattatctgatatcctttttacctcggcatactatttgaggagttggtctctagtatgcttagtgcactggtgctcctgctcattttcttttgttgggtggtgtgttgagttgtatgaggttctttaacacctcaccccatctttttctacttaatataatgatacgcagctctcctgcgtattcgagaaaaaaaagaagaaaaaccaatcacaataaaagatgacacaagagacacggtgattttatcccgtgtttgaccaatgcctactccacattgtggtgacctccttcggtccaagaattgcaactcaatccctctcaaatgatccaacgatcaaacttgagtaccaccgttttcttccttagtgatgtttcccgtttgtgaggaatctctacaagttggagcctctcacccttacaatgttgatcacaagaaaagcacaagagtaagggagggaaagaaacacacacaaaacaagaatcgcagcaacaatacacacacaagccaagaagagagcacacaagaacaatgccacaagttcacaacacactcagtggtcaaatctcaatcatgatgatccaaatgcgtatttgcggagtttaggcgtcttaggatgtttagagaatgtttggtgtagtgctccatgcgcttaggggtcccttttatagccccaaggcagctatgagccattggaattccatttggaaggcaattcttgccttctgtcgggcggcacaccggacagtctggtgcaccaccggacatgaacagtccttgttcggtgcccgatctccttccttttctggcgaagccgatcgttgagcccttggtccccttgtcgcaccggacattgtccggtgcgaccaagttaccgttggcccaggccacgcgtcacccgctgattgcgctgccgaccgttggcgagggcgccgttggctcaccggacagtccggtgaattatagccgcagcgccctcggcgaattcccaagaacgacgagttcgtcgccgagccagcctgggcaccggacagtccggtgcacctcaggctggttcaagtttggctgcacttagccaaacttctccaatccaatcttagttgattaaccaaggttcctagcacttagagaaatatgttagtactcaaaaacaatttactaaggccagagtcataccttgattctttgatttgcatctcttccacacttagtacatttgaacctaagtaatttgtgttgggcatctaatcaccaaaacatttatagaaatggcccaagggcacatttccctttcacaaaaccataaagagcaatagcaaaaactacccaaatagttcaggggtaaacaaggtgaaaagataaccagactagggtgacctattgggtcccatcaaaattaaacctatgcatgaataaatgattataaagaacattattgggtaataaaagtggtaaagggcacaacttgccttcaatgagctcctgctcagtaacttctacctgctaaacaccctgatcctcggccactggctcttctactcgtcacaatacaaacaagcatggtataaggaagaaattaacatcacaccaaacatgtatacaaaatacataataataatctacacattaaaacgagatcataggaaaaagaatcactaaattcggagttacagatttcaagttatgattttccgaagtatttatgtgcttggtacaaaattgattgtgtgatcaattctaatgtgggtttcatggtaaaacagtaacactaagtgataaacaatattattacaaaattataggaactggaatggattaatttggacttaatatgaattagttttgtactaaaaatcaatttccataattatttatccaatttctctgttctctgggccgagcactaaatataaggaaacacgggggcagaatcataagatttccccagactcagagcaaccctggggtggacggcgggttgattttaagtattcctaagggctcttttgagatattgccacgcgaaggggtactgtgtgatctgagccgtccgatcaattccagacgctcttgatttaatttacccgagcgtgaaacggtacgctatggcagccattagatcgccgatcgacgacacggattcaaacaccccacgccgccaccgagacgtccgatcgggatctagcggcgcggatctgtccaatcgaaggggtacgcggcgtctaatctaagccgtccatttagcatctaacggttcacactcagtcgtctacctctcagtcgtaatagcgcgacggcggcgaattctcccgtgacggagatctctccggcgagcacccacaggcgcaccaacgccccgaagccgaaattgactaacacatacaccctacgaggacatggcgaataaactggggagtgaatttacctcggttcacggcggcaaggtctctgttcacgacacaaggcggtgcggcacccaagcgaagcaccgacgagcaattaaggttgctctgcttgtcgattcggccagcaagtgcttcggtctccctcgcagcaactcggcgcaggttcctgactcgatctccggcactggacggtgacgggaacgggaacccgtgtctccccacttctgccacccctggcttttctctccctaagctctatacgcgacggcgagggtttggcacggaagcgagaggcgcataaggttcggtgcagcagagttttatggccgggtgaggttgggcggttagcggccagctgatccacaaaccccgcgtggtggcggacatctcggccatggccgttgcggaggtgagggtgatgttttgacacggggggcccagacgcagtgagggcgcaacgcgacgtgttgagtcggtctctcggggcccacgggacagaagcttcgtgcgaccgagttacgtagactggcaagggggccccgcaagtcagtcatggattccggctgggccttgcagagaacgtgctggttgggccggctaagccggattaggcccaggcgcacagttcctttcttcctttttcttttctattttatttttcctatttctttcattttcagttttaagtttcgaatttgaattcatatgtgtggagctcatacttaggttaaatgttttcattcaaataatagtatgaatcgaatttatttatttaattaatttaatttgtatagtatttttcttcttttttctcagatcctagtattccaattagatctttaattccaaattgggccagtaatattttgctattaatattatttttattgttaccaaatgcacacacaaagaaactccgacatgatgcacgatttttttattggttattggttaattacttacttaaaataaggttgttcgcatgtgatatcaaatagaggaaaatcatatagaaaaaaattatctcctttactttgggttacaagtttgggtattacatgaaaatcatgtgtagatccttcttttgctccctatactgctctatcacttgtcttgttaagaaaactagaacatgaggcgcccgtaagggcgccctatcaggactaccgtatacaaagctgaacaatagttataaggacactaaagaaaggttcatgtatgtgaatcacatggtaacaggagtagtgatgatcgttctttattttaccaagatcgtcatccgtagccttggacttctctagaggcaacctaaaccacaacttgaaagaggcagatgaaaaatatatcattcagatcagatataagcatatctagaacaggataaactgaggaaacgaggagtgagacatgaaaggcaatacattcatatttgtcaggccttgcaaaatttgtttcacaactctagcaagatactcgatagggagggactacctctgtgcaaacatggggttgtttgcataagtagcagaccataccagtccatttaacatgtgtttagaaattaggatttcctttgcacaagaattgatcaacatgtacattggccacccgtagcaattaaaacttcacctagagacaaattgtagaatgtgatttatttatatgtagagtagagctataagtttctcgttggatactttatctgtcctaaaataggtgtatatcttgcactccgagtcgacaaattatatatttgattaagtatatgaaaaaacattaatatttatgatatcaaaacattaatatttatgatatcaaatagacatcattaggtgagtcacattgtgtgtagtaaagatattttaagatagaaatattaatactatttttctaaaaatctagtcaaagttgagaatgtctggcttctcaaaatgtgaggtctaagatgtgcacctattgtaggatggagtaactatatagggtacataaaacaaacaaacaacgtagatataccattttaaatcgaaacacaagtaaaaacaaacacctcgattgacgaatcccatttcttagtaaattagttggtggatgtgtcatgtacggtaagccgtacacaaggcgcaggcgcgctcgcgacatccggcgggcagcggagcgcgccaacagtgcagcagcgacgcagatggcaggcactcaagtttaggaaagcagcagccacctatggcaagggataaataggaaagtatcaatttggctcttatggcaaggaataagtaggaaagtagcaatttagcacttatggcaaggaataagtaggaaagtagtattttggcagggaagataattagggagcttgctccatttgtgtggccgccggccctatatatccccaagtgcgcagcactgttgagggcaataaagtatcaagtcatctcccacctctctctctcaagcctgcggttaggaggaatactcctaccggagaagacggccgaaggctacgagttacgtagccgaggcccggcgagtcaagggttcgacgctcttgacagcttggtatcccgagtccggagatccttgcctcacccagtcccgcccaccgatccactaaccacaccaccatccccgaccacgcccaccagcggttcgccatcctccatccacatggccgagcccactttagccgacgtcatcgagatgatgaaggccatgcaggccgacatcaccaggctgaaggagaagtccgactcctcctccgcaagcggcgcccagcccgagcgtcatcgcgacatcgatcgcccgccaaggttccagaaatgggactttccgcggtttgatggcaagtccgatcccctcctcttcatcaacaaatgtgagtcgtattttcgccagcagcgtacaatggtggaggagcaagtgtggatggcctcgtataatctcgagggcatcgcacagcactggtacatacaactacaggaagacgagggcaccccgtcctggagcagattcagggaactgctcaacctgcgcttcgggccagccctccgttcagcgccgttcttcgagctcaccgagtgtcgacgcaccggcacggtcgaggagtactccaaccgattccaggagctcctaccccgtgctggcaggatgacggaggagcaacgcgtccaactctacaccggcggcctgctgccaccgctcagccacgcagtgcggatactcaaccccggcaacctcaacactgctatgagcctagcgcggcaggtggagcagatggagctcgccaagatcccggcaccagccaagacagggccgcgcggcatcctgccgccgccacctccgcggccagcgactcaggcgctcccggcgccacaggcccccttggcgctgccagcaccgccactgtgcgcagcaacgggccgtggcgacggctccaggcgattatctccagaggagatggcggatcggcgccgccaaggactgtgttttaattgtaacgagaaatattctcgtggccacaaccgtttttgcaggcgcatcttcttcttggatggcgtcgaaattgaggaggccgaggcggaaacgggtgccgcggagaccgacgctccctgtttttccctccaggcagtggccggggtcgcgccaggggacaccatgcagatcgccgtcaccctcggttcagtctccctggtggcactgctcgactcgggaagcacccacaattttatatccgcagcggcggcgcaccagtcgggcatccccatccagcgccgaccccgcctcacggccatggtggccaatggcgagcggatcacctgcgcgggcgtccttcgcaatgcgccgctccacgtcaagggcgaactctacccggctgacctcttcgtcatgccattggccgggtacgacgtcgtcctcggcacacgatggcttggtgcgttggggccaatcgtgtggaacctcgccaatcgccgcatgacgttccagcgccaggggcgctccatctcctggacgggcacgcccagccccaaggagccggcgctgcgcgccaccaacggaacggacgccctactggaggcccttctcgcctcgttcgggggactcttcgcagctccgatgggtttgccccccaagcgcgcccacgaccatcgcattctcctcaagcccgatgcgtcaccggtggctgttcggccgtaccgctacccggctgcacacaaggacgagctggagcggcagtgcaacgccatgattgaccaggggatcgtgcgtcgcagcgactccccattctcatcgcccgtgctcctggtcaagaagcccgatggctcgtggcgcttctgcgtcgactacagggccctcaacgccctcaccatcaaggacgcgttccccatcccggtcgtggaggagcttctcgacgagctccacggcgccaaattcttcaccaaactggatctacggtcgggctatcatcaggtccggatgcggccagaagacgtgcacaagacagcgttccgcacacacgatggcttatatgagttcctcgtgatggcgttcgggctctgcaacgccccagccacgttccaggcactgatgaatgacgtgctccggcccttcctccgacggttcgtcctggtattctttgatgacatcctcatttacagcagcacgtgggccgatcacctccgccatctacgggccgtcctcgacgagctacaacgccatcagctgttcatcaagcgttcgaagtgttctttcggagcaacatctgtcgcctacctcggccacatcatctcggccgcaggggtggccatggacccggccaaggtggctgccattcacgactggccgccgccgcgctcggtccgggccgttcgagggttcctcggcctcaccgggtactaccgccgcttcgtccacaactacggcacggtggctgcccccttaacggccctcctcaaaaaggatggcttcgtctgggatgacgcggccatggctgcctttacggcgctcaaggcagccgtgtcgtctgctcctgtcctcgccatgccagatttcggcaagcggttcgtggtggagtgcgatgcatcctccacaggcttcggtggggtgctcgtccaggagggacacccggtcgcgttcttcagccgacctattgcaccccgccaccgcgacctcgccgcctacgagcgcgaacttatcggcctggtccaggctgtccggcactggcggccgtacctatggggacgacacttcatcgtcaagacggaccacttcagtctcaagtatctcctcgaccagaggttggccacgattccccaacaccattgggtgggcaaacttctgggattcgattttacggtcgaataccgctcgggcgccacgaacaccgtcgccgacgcactatccaggagggaccccgacgagggcacactgatggccatttcagcaccccgtttcgacttcgtcgagcgcctgcgccaggcacaggctacggatccggctctcgtcgccctctacgaagagatccgcgtcggcacgcgcgctgcaccatggtccatcatcggtgggatggtcgccatggaggggcgcctctatatcccagccacttcacccctattacgggagatcgtggccgctgtacatgatgacggccacgagggcgtccaccgcaccctgcatcgactacggcgcgactttcattttcccaacatgcgtcgtattgtgcaggaattcgtcagggcgtgcggcacatgccaaacatacaagtccgatcaccttcgtccagctggtctcctacagcccctgccgatcccaaatggggtgtggtcggacatcggcatcgacttcatcgaagctctgccccgcgtgcaaggcaagacggtcattctcactgtcgtcgaccgtttcagcaagtactgccacttcatcccactggcgcacccatacacggcggagtcggtggcgcaagcattctgcgccgaggtggtgaggctgcacggcatcccgcagtccatcgtatcggaccgagaccccgtcttcacgtcggccttctggagggagctaatggccctgactggaaccaagttgttcatgtcctccgccttccatccgcaaactgacggccagtcggaggcagccaaccgcgtcatcatgatgtaccttcgctgcttcaccggcgatcgaccccgacagtggctccggtggcttccatgggcggagtacacctacaacaccgcataccagtcctcactccgcgagacacctttccgggtggtctacggccgcgacccaccgaccatccggtcctacgagccgggtgacgcacgcgtgcccgcggtggcacaggagatggaggaacgggctgccttcctcgacgacgttcgctaccgtctccatcaggcgcaagaatcccagaaacgcgcctacgatcagcatcacaggcaggtcgtctacaatgtcggtgactgggcccttcttcgtctccgtcaacgaccatcggcatccctgccacgttccaccacgggcaagttgaagccccgctacgtcggcccctaccgtgtcaaggagatcatcaacgaggcggccgtacgccttcagcttccatccgacgccaaaatccacgacgtcttccatgtgggcatcctcaagaagttcgttggggcgccaccaacggaaccacctgcgctaccggccacactcaacggcgcagtggtgtccacaccttcccgtgtcctggcgggtcgcctggctcgcggcattcggcaagttctggtacagtggcacggtgaaccagtgtcgtctgcgacctgggaggattttgaggatttcagggcaagcttccccgctttccagctcgaggacgagctgactttcgaggccgggagagatgtcatgtacggtaagccgtacacaaggcgcaggcgcgctcgcgacatccggcgggcagcggagcgcgccaacagtgcagcagcgacgcagatggcaggcactcaagtttaggaaagcagcagccacctatggcaagggataaataggaaagtatcaatttggctcttatggcaaggaataagtaggaaagtagcaatttagcacttatggcaaggaataagtaggaaagtagtattttggcagggaagataattagggagcttgctccatttgtgtggccgccggccctatatatccccaagtgcgcagcactgttgagggcaataaagtatcaagtcatctcccacctctctctctcaagcctgcggttaggaggaatactcctaccggagaagacggccgaaggctacgagttacgtagccgaggcccggcgagtcaagggttcgacgctcttgacaggatgcgagtcaaaatcatgtcatagtagattccatctaactcaccagcacagctcaaacaaaaaatgtaatggacaataaggttattccaaaattctaattccagagtctgatcaccttcgtatgggtagtcccgatagcaacgaaactctccttggtggtggcctcatcgtctggtgtcttgcatggcagcatgctgaatgtccagccttcaaggatgcatgtcctctgggtcatccccttaaacaccatcagttgtaggaagatgaacctcagaagcatcaactctgcatcacacagagacaaagaggtaaggtttgtgccgtaaacatatgtatcttatccggaatcatagagaaaaggaaaggaagactgagggtgtgtttggattattgcatacctgaaaaaagttacgtcttgttgctgtcaacatttttaaatagccaaccgctcagtttcattttggttcaattgtacgaaggacaatcagtgtccggtgttcttgatagtcaaggcttagggcatacaagttttatcctcgtaaaactgactttaccttacattgttttgccatccttttttatctgtattatatcgttatctatgatttacttatcttgaagcgaaaatataaagattgggaaatggaggcaccttctttgaccttgagcagctcctcgaagagcggcttttggttcttcctcttgagcgtctacaggacacatcatcgaagcgctcctcgaaggcgaccccttatcttgaagcgaaaatataaagattgggaaatggaggcaccttctttgaccttgagcagttcctcgaagagcggcctctggttcttcctcttgagcgcctgcaggacacatcatcaaagcgttcctcgaagatgacctcaccggaagcgaggaacaacaacgacaagagggcgaggaggcaatagagcacgccgccacgctggggcgtggagcccgcggtggccatgcgcccgcacgtgctcaaggtcccaagcagtggggatgtcgcgcgccgccgcggcctcgggatctgcatgctcgtggggacgggcgccacgtcgtcgtctcgcgctccggctccgtccgcggaggaggctatgcgaccgtctttcgtggccagtacgagatcctatccacctcggtcgccgttgccgccatcccctgttccctagagcgttccacgaaggtgaaggaaacccttgatgcaggtgggaagagatcgcagacatcaaggtaggtggagggagcaatagcactgtagcagtagcatactactcggcttggctgttgactgttgaggaggctagaacttttcggcacggcaagttttttaatataaggattcatgagaggacatgcatgccgaaatgtttgaccagctgaacaattcgcgttcgtggtcgctttccacacgtcgggaccggtgtggggaaaatcacgcgaaacgcggagagcgttccaaaacccatcgtattttaatactttagatggctttcattgttgaccttccaggcatgaaaccaaattggttcaaagagatcctcgttattgctttcaaacgatgcttgataactctcccccatagcttcatagtatgactcatcaacttaattccttggtaattattacagctttaaatatctcccttattcttgtaaatcggtacaaataatacttctccacttgttaggcgtattgttcgatcgaaagatatggttgaacaacttggttagccatactatagcaatatcctcgaggcatcttcatacctcaattggaatatcattaggatccatcaccttaccccctttcattcttttcaaagcctccctaaccttagattcttgaatcttacgcacaaagagcctattggtgtcatcaaaagagtcatccaactgaaaggttgtgtccccattctccccattgattaatttgtcaaagtactcttaccatatatgcctgatctcgtcctccttcaccaagaggcgctctgtttcatccttaatgcacttaactcgattgaggtctcttgtcttcctgtcacgaaccctggctatcctatatctgcccttctctcctttcttcgtactcaagcattggtaaagatccttagacgcactattctttgctaccatgtatttttctatgctatccacactcctacctactcttataaagcaccagtttccttggtttccaccattgtccccaaggttttaaagtctggcaactagtcgagaagtcgatctgaacgactcgactttctctactagtcgctcgactcgttgactagtcacgagtagtaacgactagtcgcagcgattgagtcgggtgacttatttgggcctaggtataaaaatgtgagcccatccagccatctcaaccctagtaggtatgagatggaatgagatggtatgaaatgctaatagacaagttttctaccttatttatctatgatatggaatgattcatgacttgatgaggccaagacaaatggttgagatcttgtgaggcatgcaactatgttttgcaactactatgtttgcaacatccttccttacattggctaatatgtggaaacaaagacaaggatcgaaagctctctttgttagtaatgaatggtgtgagagcaaaatgaagaacactgaagtaggaaatgcacgtgttgtttgtcactctgttgcattttggcaagcagtagaagattgcatgagagcttcacaacctattctaatattcctgaggatagttgatggtgatgagagactggcaatggctatatggtgggctgccatggaccatgaaaaaagagcatcaaaaaagctttggagcacatagaatcatagaaagaataatagatgaagtaatttctattgttgataaggggtgggacagtcagatggataccaatctgtatagagcaacattgtttttggatccaaacaagttctttgaaaccaaagagaacgatagaagacttactacaatgctatattcaatatttaatgatatattgtagaagatggagatggatgaaaatatactaaacaaggtgtttttagtgttctagcatgtttttagtcttagcgtatgtagaaagttataaaaatataatcactgacttatgttcgactagtcacgactagtttgtcctagtctcgagtctctactagtcagtagttgcacgactcgactcgaaaacatcgattgtgtgggaaaccctaaccctaacaaggggttgggtgatgtattaatagactgggtagttgggcctgacccattacacaggggtgagaaggtaaatacacggggtagaccccaaaccctaaacgagtactctaatactctaacagattgtcccttttgcaaaaaaaggtccttatacttctttctaatctaactgtagcccaacttatagatcttgatttcctcccccatgtcttgcctaacaaccaaataagtgtgtgggtgtgtcacgctcgaagcgtttagatctaatttcctctccttgtctggcgtaacaactagataagataagtcttaccccttgcatgcacaaccaaccaccacgagtgcttcctcctcctcgttattggtgtcgtatattaacccctaacatcacatctctttccccatccctgcctttctcacccaaattcttccgtggaggacatgcagtaggccatcctgctggcctccaactcgccagtcgctgtctccgcgccctaccggcgccgccgcctttaccttcccccgtcggcaagcgcctcgatcttgtagcgcctcctctctttgccgccatgcgcctttgcagtcagcggggcaagaacaaccgcgggagagtaggaggcgttgccgccgctggcggtgcgtggccggacaattgagggagaggaggcgctctagattccgagtttagttttgtattggttgttggcaaaaaaattgtgagatattaaggtcgatgataatatatgtattgctcgttttcatgtaatcgttgtgcgctaatattttatcgaatatttttgtgccgtcacaacacacgtgcattatactagtcatggtacaatgttagcatttttttcttctccttaatagccctttagacttcctcattccactaccaagtatctttagcttcgcctctattctctctagttactccacacacctcttaggtggcctttcgaatgcaggttttcatcttctcccacatgttgtttgtgtcatcttccttccaagtgtcctctttgagaagcaagatgacacaaacattgaacttgcattgtattttatggtgtttggcatgtgaattcatttagctgagagttgattgaattggactacaattggacttccgtttttttacacacatttgggaggggggcgggagggcagacccacccagtggtggaggttcccacatgagtggggttggggaagggataaaccaaggcaagccttcccctcgctaatacggagatgctgattcgaacccgtgtcctcgtgactcactgggacatctctcaccactgcactaggcctgcactttatttgtttttctcatttttttttgttttcctaacttgttcatagttattgtacttcccgcttttttacacgttacaggaattgttgttttttgttcttttgaaggtaattgcaatagtagtttatgtagcttgtcaactgtattctcactgcttacatgtgctttctacacaggatttcttacatgagatgtcacggaaggacgaaaaagttgattccttagttaaaatggggtttcctgaagacgaggctgcactggctattaccagatgcggtgtgctctctgagactttgttccattctcgtctctttgcattagattcctcagcaaaataaacatcaatttccaaccttgaaagtaggatatgctctcatatagaataaatcagttgaataataacattgccatgtttaattatatgtcaagctttctattggtttactctctggattttgagaattctcttcaaatgctgtaacttggaactcgtaaattggctgcaatcttgacctgaggtaacctagaatggttgaatctttccaagcttgatttactgtttgcatcaagtttgggttaaccagttatattcaaatctgaagacaaagcatttaggcatgcccataataatctgtagaaagcaaggcgtatgataaatattatttcttacaagaataactgggtgtttaggaagttatccatccaagtagtacatgtaaacctttgtttctatgagtagcaagcattcagatgctccttcaaagcagtcaacgaacagtttattgttcgtttggggtcagaatattttaatggcatttgctgttaaataaaacctttttttgcatcttgcatgcaacttgtaccgctcagattctgcccccgtccctgttttattttaactggccatgcaccttacgccatctactgtggtctgtggttgaaaattactcagggccggatgcatctatttctgttctggtggattcaatctatgcttcacagaccgcaggagatggttactgtggcaatctgtctgactatgaggtttgtcttactgtccatacaagtctgcaataagatttagttctattataatggcctggtaatggaactgtgtctgcaatactgcaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccgccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacagatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagttcttagaatctttactgttgttttggttctagtagaatactttgtgaccgaacttgtaaatggtttgcaggttgactggtttagttctcttgccactctaatttaggctagttttttttttatttaatcttctttggtgatcttggtgcagttctgtggcactgttgggtattcaaatgcagattgatccgagaggctgccctgttgcattttctttttaaaatttgtatttgtcaatgagagatcaggtattctggttgccaaaaaaaaatctgaagttgtggagctgagaaaaggacggtgctaagctacaaccacccactgccacaccatgcgcacccccaacccaaaagatcctgcttttgctcacgcttatcaccactcccatctccatccaaaaccataaaaaaaaccagtgcgagctgaagcatccaggctctggaatcatcgtcgggatgtctgtgccagcatcagcccaggtgcctcacaccatacgatctgctgggagctgctgcttcaggtcgtcgcatcaactatctgcgcagaatctgggccgacgtgagttcccgatctcagctttgagtggccttctttttgaaagtgagcacaggttcttccaaagcccgttgtgttcgtgtagatcgcaggtggcgggggaccggaaagaagaagatcctttccacccgtggcgtgcttgagtccagaaacggcgcacatcctggtggccttatgaagagaaggtgagaacctggtgaaagacatgcggagctgtagctagacagctagctgatctagtagcagcgcacgccttgcaggaaggtcgtggagcacatcatcctgctcagagccaagcccaacatctcagacgctgaagagaaagacatgctggactacctatacacatcg

SEQ ID NO: 3 - DNA, 2353 bp, Artificial sequence
  Methyltransferases
gagtcaccctgtcgccgttgccgtcgccgaggcaggcagagtctccctgtcgccgttgccgtcgccggcctcctcctcctctgccacttcctcccaactcccagccgcaggggccgacggcgacgggagggaagaggcggcagaggtactccgaggggcgcggagagaggctgtgccaggccacgccgtccagtgcctccgggtccgtcgccgcagcttccggccgcgtcggagaggtagcccccggagctcttcgcggaggctcgggaatggtgcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggttgctaatggcaatgggaaggctgggcgatctgcctctttggttcagaagtatgtggacatgggtttctcagaagagattgttctgaaggccatgaaggacaatggggataatggagcagattcattagttgagctccttcttacttaccaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggatttcttacatgagatgtcacggaaggacgaaaaagttgattccttagttaaaatggggtttcctgaagacgaggctgcactggctattaccagatgcgggccggatgcatctatttctgttctggtggattcaatctatgcttcacagaccgcaggagatggttactgtggcaatctgtctgactatgaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccgccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacagatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagttcttagaatctttactgttgttttggttctagtagaatactttgtgaccgaacttgtaaatggtttgcaggttgactggtttagttctcttgccactctaatttaggctagttttttttttatttaatcttctttggtgatcttggtgcagttctgtggcactgttgggtattcaaatgcagattgatccgagaggctgccctgttgcattttctttttaaaatttgtatttgtcaatgagagatcaggtattctggttgccaaaaaaaaa

SEQ ID NO: 4 - DNA, 1812 bp, Artificial sequence
  Methyltransferases
atggtgcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggttgctaatggcaatgggaaggctgggcgatctgcctctttggttcagaagtatgtggacatgggtttctcagaagagattgttctgaaggccatgaaggacaatggggataatggagcagattcattagttgagctccttcttacttaccaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggatttcttacatgagatgtcacggaaggacgaaaaagttgattccttagttaaaatggggtttcctgaagacgaggctgcactggctattaccagatgcgggccggatgcatctatttctgttctggtggattcaatctatgcttcacagaccgcaggagatggttactgtggcaatctgtctgactatgaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccgccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacagatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtag

SEQ ID NO: 5 - protein, 915 aa, Artificial sequence
  Methyltransferases
MAPSSPSPAAPTRVSGRKRAAKAEEIHQNKEEEEEEEEVVAASSAKRSRKAASSGKKPKSPPKQAKPGRKKKGDAEMKEPVEDDVCAEEPDEEELAMGEEEAEEQAMREEVVAVAAGSPGKKRVGRRNAAAAAGDHEPEFIGSPVAADEARSNWPKRYGRSTAAKKPDEEEELKARCHYRSAKVDNVVYCLGDDVYVKAGENEADYIGRITEFFEGTDQCHYFTCRWFFRAEDTVINSLVSISVDGHKHDPRRVFLSEEKNDNVLDCIISKVKIVHVDPNMDPKAKAQLIESCDLYYDMSYSVAYSTFANISSENGQSDSDTASGISSDDVDLETSSSMPTRTATLLDLYSGCGGMSTGLCLGAALSGLKLETRWAVDFNSFACQSLKYNHPQTEVRNEKADEFLALLKEWAVLCKKYVQDVDSNLASSEDQADEDSPLDKDEFVVEKLVGICYGGSDRENGIYFKVQWEGYGPEEDTWEPIDNLSDCPQKIRDFVQEGHKRKILPLPGDVDVICGGPPCQGISGFNRYRNRDEPLKDEKNKQMVTFMDIVAYLKPKYVLMENVVDILKFADGYLGKYALSCLVAMKYQARLGMMVAGCYGLPQFRMRVFLWGALSSMVLPKYPLPTYDVVVRGGAPNAFSQCMVAYDETQKPSLKKALLLGDAISDLPKVQNHQPNDVMEYGGSPKTEFQRYIRLSRKDMLDWSFGEGAGPDEGKLLDHQPLRLNNDDYERVQQIPVKKGANFRDLKGVRVGANNIVEWDPEIERVKLSSGKPLVPDYAMSFIKGKSLKPFGRLWWDETVPTVVTRAEPHNQVIIHPTQARVLTIRENARLQGFPDYYRLFGPIKEKYIQVGNAVAVPVARALGYCLGQAYLGESEGSDPLYQLPPSFTSVGGRTAGQARASPVGTPAGEVVEQ

SEQ ID NO: 6 - DNA, 10088 bp, Artificial sequence
  Methyltransferases
ccggtgtgcacccgacagggggacaatttgtttttcaaaacaaactagtgtatgctcctactctgttagctgtacttaacattgttaagtgttgataaaaatacaagagataattgcgccaatgcccctgttttagcgcaattgtattctactatatttttagacttacatgttttatgacaattcaaattaatcccacaacgtttcaaattattttaacaacaattcaaactatttgcaacaatttaaattatttttaaagatttaaaaatattgacaaattattccatcaacattttaaattattttgcctcatcacactaatttaattagttagatattttttaactttgaaaataataatttgaattgtcataaaagcggacaaagtctaaaaaaagcggtaaaactatagttacacaacaaaacagtgacaatggcgcaattgtttcttgtcttctttatcatgacttaacactgttaagtataaccaacggagtatgggcatacatcgttttattttgaaaaacaggggcaaacgcgcaaagttttaaaaagggggtaattctacagttacaccctaaaacagggacaccatcacaattgttccttgtttttttagtaaagttttgttcatgtcacagctgatgtatgaatatcaattgtaagtactaaacatagtataattacaaaactaattacacaaacaaagactaaattgtaagacgaacctgttaagtctaattaattcatgattaacaaatgtttactgtaatatcatgttaccgaatcataaactaatcagatttaatatgttttccttattatgtgtgtaattagttttataattatattatatttaatatttgaaatgtgacaatactaaagctaagcctcttaagcccaacaatccacatattttagatttaaaatttgaaaacagacgtatcggctagaagagccctgtcactgtcagctaatcaattacaagaagtggcccatactagttccatcaccagtccagtagtccaccaccccaccctacagctgggtcatctggcacgggtggaggggccaacggccgaaagcgccgcgcacttccggcgggcaccctcgcggagtcgcgggtgacagcgaaatttcaaatccataccctcccgctgcagacgggtcccacgccgtcaaaatttggacgctcccgctccctcgatcttttgggtttcgttttcccagttcccaccctctcttccaccctgccctgtttccagatttgaccgatccccttcgattcgatttctacacctccagagcactcactctcctggcaaaccccttcgtctcccaaccctagagagcagcagcagctaccgcagcccctgccatggcgccgagctccccgtcacccgccgcgcccacacgcgtctctgggcggaagcgcgccgccaaggccgaggagatccaccagaacaaggaggaggaggaggaggaggaggaggtcgtggcggcgtcctccgccaagcgcagccgcaaggcggcatcttctgggaagaagcccaagtcgccccccaagcaggccaagccggggaggaagaagaagggggatgccgagatgaaggagcccgtggaggacgacgtgtgcgccgaggagcccgacgaggaggagttggccatgggcgaggaggaggccgaggagcaggccatgcgggaggaggtggttgcggtcgcggcggggtcacccgggaagaagagggtggggagaaggaacgccgccgccgccgccggcgaccacgagccggagttcatcggcagccctgttgccgcggacgaggcgcgcagcaactggcccaagcgctacggccgcagcactgccgcaaagaagtacagtttttctcccagctctggttttgatttgaccagatttttgctccatgtctattagtacttgtgagccgagcaatctgctatttgctgatttattgtgcgtgcagaccggatgaggaggaagagctcaaggccagatgtcactaccggagcgctaaggtggacaacgtcgtctactgccttggggatgacgtctatgtcaaggtccttgttcatcgctttctgttgcttctgctctcatttatgatgtgcatatgtgtttgttaaggaagcaagaattgcttgatttttgttgccgactcgcatttccgtgacgagttctgcgtatggtcaccggtacgtggcactgatacacaacgtggtatgctggaagtctggtagtatattttgcatcgaccaggaggtccagatcgatatgtgcggtatagtgcttatttgattgcaccctgttcggagattcatgcatgatggcgtgtttagatgacgcctcccagacagctgcctgctaggcagctgattctggcccaggcgtccggaatggtgaagttgcgctggcaagattctcaggccacctaccaaatatgccctggagcatattgcatgcttctttttttgttctctttccttctatatttatctcattgttagtgaagtttcacattgcgcgtgtcatggaatatttactttcaaatcaacgaggagatgctagcattgaggtgtgtgataattattacatactagaagatatcgtgcatgttgccattgggattgcgaagaatgtggaaatgatgttggtagcttgtattaagaggttaacagttagtgggatgacatgactattagttagagatgatgtggatagtaagtgggatatgatgtagatgacttgtgtgttgagacagaactataacatggagttggaaatgggagcagcatggtcaaacataccctaaatgcctgtctctacacaatgtggtgattggtgtatagtctggtgttaaaagctggatactttgattctgttgaagattgtcacacccgaatttaaggacaaatctagatacatctcatatgttcaccaggatagtgtatagataccaatgtcataatctttattacacgacgataatgtcttacaaaatatctggtgttacaagatgcacctttcaacatgtttaatgctgcaaactgttttaattaaacagaatgcagtgttttgaacaaaaaaatgctgctttatcctgcatcttgttttgcatgtgtcagcaattgcttacattccattatgatctctgagattctttaaatttctagcctgatgaaagtatttactaattcaactgaacacaaacattgtttgaatgaacaaggcaacacggatgcttggaataatggttgtgtataatatcacttagtggttttgctctcacaccacatctttcatgggttctataataatagttactgactttaagtttcttattcctttttgtctatcttaggctggagaaaacgaggcagattacattggccgcattactgaattttttgaggggactgaccagtgtcactattttacttgccgttggttcttccgagcagaggacacggtgtgtatttagtattttgtcattctatgcatgtcaagtggcatagcactcacatttgtcatggcaggttatcaattctttggtgtccataagtgtggatggccacaagcatgaccctagacgtgtttttctttctgaggaaaagaacgacaatgtgcttgattgcattatctccaaggtcaagatagtccatgttgatccaaatgtaagtttgctgcagtttgctgagagctttgtggttttgctatacacataatgtttctgactaccattgttttgttgcctacttgccttagatggatccaaaagccaaggctcagctgatagagagttgcgacctatactatgacatgtcttactctgttgcatattctacatttgctaatatctcgtctggtaattccttctgcatcatcttttttggttgactagctgaatgcagttagctttgccaaagagttaaatacatgagttgttcctgcactcgtcaagggatgtcaataatgtccacaaactctgaaaatgtatttttagatacttaacttgttaagtcagtaaaacctgtcagatacttgggttttgggtacgattaccatccttatgtgagtaaaactcgtcaagggatgtcaatgacgtgttgattgtgtattagatattctgtttgttcgaaagctaatctacacaaacagcttatgtaatgtaaaacctcaaacaaacttgcctcttcataagcttaggtttataggattagcgtttagtgcatgtaaggcctatttgcttcacggcctccctgccgagctcctggctagacagccatcctggccgtaggtgcccgaaatcgaacacctgggagccacgtttgcactggcaggttttcctgggtgcaaaccaaacacgcctatagtgttcaagtataactgaattggtgctcacctttgtctaatagcttaagtttttggttttcatcggtgcatgcaactccatactcaatagtcaatatgatatagtgttcaagcatagaactctcgagtttgaatcctggcaggggcaatcaaataaaataattgcagcttacccctatttccacgtttgagcacatgagggagagtgttgaattataagtgtgttctccatctttctctaacagattgaactggtttgtgcatgcaactcaatatgatatttgagtcaaatgtttactttaaaatcatagttgatgcaatttaataacatatttttttggtctcgtgtgagggagtgtacgtataactgaattgcacacatttccttatcagcttaggtttttgactgcaactgttggtgcatgtagctcaataactaaagttgatctggacagtctacagtgaataagtttgacacttgtaaaatgtgcatgtatttttacaaacgctggcacttttttcctaatagaaaatgggcagtcagacagtgataccgcttcgggtatttcttctgatgatgtggatctggagacgtcatctagtatgccaacgaggacagcaacccttcttgatctgtattctggctgtgggggcatgtctactggtctttgcttgggtgcagctctttctggcttgaaacttgaaactgtaatcttctaactagtcatctgttggatagaatatgttcacgatctcagaacttattctattgttctggcttgcagcgatgggctgttgatttcaacagttttgcgtgccaaagtttaaaatataatcatccacagactgaggtatggatagtaaacttcatcttggattccatctgttctgtcagctactcttacaaagtgtctggatttttggatgtaggtgcgaaatgagaaagccgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtacttcagtgtcatttgttcatttctacttgattccaacaaaaaaatcaattacttaagcctgtcaaacgatggatatttctgtatattttgctgtaacgctagatttctgcaggtccagtgggaaggatacggccctgaggaggatacatgggaaccgattgataacttgaggttagtgtatggtatatcgtctgcttgttgccttgtatacctatttgcatctaatccttgttttgcaaaccagtgactgcccgcagaaaattagagattttgtacaagaagggcacaaaagaaagattctcccactgcctgtgagtatttagttcgttgtgattttgctcgctatttgtttagctccccttttttatttggtgatatctgcctattttattctttcaaagggtgatgttgatgtcatttgtggaggcccaccatgccaaggtatcagtgggtttaatcggtacagaaaccgtgatgagccactcaaagatgagaaaaacaaacaaatggtgactttcatggatattgtggcgtacttgaagcccaagtatgttctcatggaaaatgtggtggacatactcaaatttgcggatggttacctaggaaaatatgctttgagctgccttgttgctatgaagtaccaagcgcggcttggaatgatggtggctggttgctatggtctgccacagttcaggatgcgtgtgttcctctggggtgctctttcttccatggtctgttctgtaccttgctgttttatatgcttcgctagattcatattgcactgttggctgctggctaaccaggtgtacgtgtatttgacaatttaggtgctccctaagtatcctctgcccacctatgatgttgtagtacgtggaggagcccctaatgccttttcggtgagtgcaatcacaaaccactactatgaaatcatgtggaatgtgtaaaatacgctgaccaactgaatttgttgcagcaatgtatggttgcatatgacgagacacaaaaaccatccctgaaaaaagccttgcttcttggcgatgcaatttcagatttaccaaaggcaagtgttctgtcaagttcatgcatttctcagtgagcatgctatttaactcttctctgcaggttcaaaatcaccagcctaacgatgtgatggagtatggtggttcccccaagaccgaattccagcgctacattcgactcagtcgtaaaggtaaaaaaccccgtgaactactactggttggccttcactacgaatatgttaggatttaatttcagaagaaccgccttttttttcttggtgcttcggtactactgcagcaagctcactcttattatcatgtcagacatgttggattggtccttcggtgagggggctggtccagatgaaggcaagctcttggatcaccagcctttacggcttaacaacgatgattatgagcgggttcaacagattcctgtcaagaaggttggtggcttggtcgcatttgtgccttcctttgttgttttttccccttctgaaacaatcatctctctttcctatgacagggagccaacttccgcgacctaaagggcgtgagggttggagcaaacaatattgttgagtgggatccagaaatcgagcgtgtgaaactttcatctgggaaaccactggtatgtgtgctatttcgtgctgttgtttctataactgtgcaacatttactttccatattcaaactcataactgacgagatgctgcaactactgtaagattcatggctaacccatgacaacattttgcacacatctttgttatctaggttcctgactatgcaatgtcattcatcaagggcaaatcactcaagtaagtttcaaaacattttttttgtttttttggggaaaagtaggttattgtttacttgtgcttacatatgatgttgcaggccgtttgggcgcctgtggtgggacgagacagttcctacagttgtaaccagagcagagcctcacaaccaggtcagcttcagaaaggccactccttttcgccaatccctgcatctgtatttactattagcgtgtgttcccatatgatcattaccgaacatgttgtccacacaggttataattcatccgactcaagcaagggtcctcactatccgggagaacgcaaggttacagggcttccccgattactaccgattgtttggcccgatcaaggagaagtaagttcctgttttcaagttgcctgtaccagatctagtcactattgaaagttttcagcagcaagccattcatcagttagttacagctcttgaaagccttacctctgaacatgtgtgctttctctgatggtgataggtacattcaagtcgggaacgcagtggctgtccctgttgcccgggcactgggctactgtctggggcaagcctacctgggtgaatctgaggggagtgaccctctgtaccagctgcctccaagtttcacctctgttggaggacgcactgcggggcaggcgagggcctctcctgttggcacccctgcaggggaggtagttgagcagtaaaaggatgacagatctgagctgagctgggcaacatccagcggcaggagcatttctggttcggttcgattcgggctcacgagcgcattgtgctgattcgcagggtgcactcgagctcgagtgcggagttgttgtaatgtaggttgaggtttcaaaacatccatccatccatcggccccgcctatttgtggatggtgtttctcgtgccattttagttcagctgtattgatgcttcgtcatgtatgtgtattgtaccattcgttcttgttacctactagtaaggcaacatttgatccatgttcaacgcgagcaaaattccatccactttaggtactgagatgctcaagtagttcagtcatcaggtaacatttacaacctattattttgcctggtattatctgaaaacgatttgctgaggatgttttttttttgaagtaaaggtactgtaggaaggtcctacagtaatattattaaatcaaaagaactaagtttagggagacagaagaatattacaaagaaataaaagggagagtgaggagggggaagaattctggatacatgtatttactctatttcacataaacatatggacatcaacatagatgtgaaggtatctaatctaaataagcgccgacatgctttacaagatagtttgatctataaggtgagggatgttgtggatgaagattgtgaccaactagctcatgtgtagttttgtttgtagtacctgtcctaaaaaatatgtaattctaagtttaatttgaatcagccttgttttttaaaatttttttgaccaagtccgttaaaaagtaataacattttttacttcaaatagatttactatgaaattttgtcttatggttatctaatgacatttatttaaaactgtaacaaaaactggtttggttaggggactgagggagtagtgcttaaaatggaagacgataggagagatcacattagtggtggcggtactgacagtgtcacgcggtgatgttttgcagtgtgtttacttcgccctctaatctaagaaaacaagtattctgtcttcacctattcagaatcttttgatctaccgcttatggctggtttaatgcatgtgtaccgagttcaatataggccttgtttggttgtaatcggatagaggatcgaaagagattaaatctctttttatttaattttgactagtaacaacttccttcaatcctcttcactcgatgaacccttacatgattaacgtgtatctatgggggtgtgttgaagtaaaatattttgtggagttttgaggtaatgatttcacgagatgtggatgccgaaggcgcggttgcccacgcaataaccgttactggaggtaagtttacgggagagcatagaagaattagttcacattgtctataggggtgcaaaggcacttcccaaaaaatgtaaaatttataaatttagttgaaattttattatatatgcacctctagaacaaattattatgcacccacgacacatacacccctattaaatttacgcttgcttcgccacggcttatgtagatacgtagctgttatatgttttgttagtatacacatgcagtttctcttatttctctgaacttcaggctagttgcaactgccggcatccaatttgtcatgatttgcacacatgttgaggtgaaccatttgcatagagttgcaacagtggggagacgcaaactgttaaatctttaatatgacccttttaatcgacaacctgaatagacagcatgtaaatatttggcaatacgcacctcttggttgggatcccataccgatctcaactatttctaatacaaattggacgacaacagtaataaatatttgaatttcttcaactaaatggactgctcagccgcttaaggccatgttcggttctaccccaatccatatggattgagagggttttaatccctagtaagtgaaaatctcccccaatccatatcaatcccctccaatccatatggattaaaaataagcgaacaagccctaa

SEQ ID NO: 7 - DNA, 3197 bp, Artificial sequence
  Methyltransferases
agagcagcagcagctaccgcagcccctgccatggcgccgagctccccgtcacccgccgcgcccacacgcgtctctgggcggaagcgcgccgccaaggccgaggagatccaccagaacaaggaggaggaggaggaggaggaggaggtcgtggcggcgtcctccgccaagcgcagccgcaaggcggcatcttctgggaagaagcccaagtcgccccccaagcaggccaagccggggaggaagaagaagggggatgccgagatgaaggagcccgtggaggacgacgtgtgcgccgaggagcccgacgaggaggagttggccatgggcgaggaggaggccgaggagcaggccatgcgggaggaggtggttgcggtcgcggcggggtcacccgggaagaagagggtggggagaaggaacgccgccgccgccgccggcgaccacgagccggagttcatcggcagccctgttgccgcggacgaggcgcgcagcaactggcccaagcgctacggccgcagcactgccgcaaagaaaccggatgaggaggaagagctcaaggccagatgtcactaccggagcgctaaggtggacaacgtcgtctactgccttggggatgacgtctatgtcaaggctggagaaaacgaggcagattacattggccgcattactgaattttttgaggggactgaccagtgtcactattttacttgccgttggttcttccgagcagaggacacggttatcaattctttggtgtccataagtgtggatggccacaagcatgaccctagacgtgtttttctttctgaggaaaagaacgacaatgtgcttgattgcattatctccaaggtcaagatagtccatgttgatccaaatatggatccaaaagccaaggctcagctgatagagagttgcgacctatactatgacatgtcttactctgttgcatattctacatttgctaatatctcgtctgaaaatgggcagtcagacagtgataccgcttcgggtatttcttctgatgatgtggatctggagacgtcatctagtatgccaacgaggacagcaacccttcttgatctgtattctggctgtgggggcatgtctactggtctttgcttgggtgcagctctttctggcttgaaacttgaaactcgatgggctgttgatttcaacagttttgcgtgccaaagtttaaaatataatcatccacagactgaggtgcgaaatgagaaagccgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtccagtgggaaggatacggccctgaggaggatacatgggaaccgattgataacttgagtgactgcccgcagaaaattagagattttgtacaagaagggcacaaaagaaagattctcccactgcctggtgatgttgatgtcatttgtggaggcccaccatgccaaggtatcagtgggtttaatcggtacagaaaccgtgatgagccactcaaagatgagaaaaacaaacaaatggtgactttcatggatattgtggcgtacttgaagcccaagtatgttctcatggaaaatgtggtggacatactcaaatttgcggatggttacctaggaaaatatgctttgagctgccttgttgctatgaagtaccaagcgcggcttggaatgatggtggctggttgctatggtctgccacagttcaggatgcgtgtgttcctctggggtgctctttcttccatggtgctccctaagtatcctctgcccacctatgatgttgtagtacgtggaggagcccctaatgccttttcgcaatgtatggttgcatatgacgagacacaaaaaccatccctgaaaaaagccttgcttcttggcgatgcaatttcagatttaccaaaggttcaaaatcaccagcctaacgatgtgatggagtatggtggttcccccaagaccgaattccagcgctacattcgactcagtcgtaaagacatgttggattggtccttcggtgagggggctggtccagatgaaggcaagctcttggatcaccagcctttacggcttaacaacgatgattatgagcgggttcaacagattcctgtcaagaagggagccaacttccgcgacctaaagggcgtgagggttggagcaaacaatattgttgagtgggatccagaaatcgagcgtgtgaaactttcatctgggaaaccactggttcctgactatgcaatgtcattcatcaagggcaaatcactcaagccgtttgggcgcctgtggtgggacgagacagttcctacagttgtaaccagagcagagcctcacaaccaggttataattcatccgactcaagcaagggtcctcactatccgggagaacgcaaggttacagggcttccccgattactaccgattgtttggcccgatcaaggagaagtacattcaagtcgggaacgcagtggctgtccctgttgcccgggcactgggctactgtctggggcaagcctacctgggtgaatctgaggggagtgaccctctgtaccagctgcctccaagtttcacctctgttggaggacgcactgcggggcaggcgagggcctctcctgttggcacccctgcaggggaggtagttgagcagtaaaaggatgacagatctgagctgagctgggcaacatccagcggcaggagcatttctggttcggttcgattcgggctcacgagcgcattgtgctgattcgcagggtgcactcgagctcgagtgcggagttgttgtaatgtaggttgaggtttcaaaacatccatccatccatcggccccgcctatttgtggatggtgtttctcgtgccattttagttcagctgtattgatgcttcgtcatgtatgtgtattgtaccattcgttcttgttacctactagtaaggcaacatttgatccatgttcaacgcgagcaaaattccatccactttaggtactgagatgctcaagtagttcagtcatcaggtaacatttacaacctattattttgcctggtattatctgaaaacgatttgctgaggatgt

SEQ ID NO: 8 - DNA, 2748 bp, Artificial sequence
  Methyltransferases
atggcgccgagctccccgtcacccgccgcgcccacacgcgtctctgggcggaagcgcgccgccaaggccgaggagatccaccagaacaaggaggaggaggaggaggaggaggaggtcgtggcggcgtcctccgccaagcgcagccgcaaggcggcatcttctgggaagaagcccaagtcgccccccaagcaggccaagccggggaggaagaagaagggggatgccgagatgaaggagcccgtggaggacgacgtgtgcgccgaggagcccgacgaggaggagttggccatgggcgaggaggaggccgaggagcaggccatgcgggaggaggtggttgcggtcgcggcggggtcacccgggaagaagagggtggggagaaggaacgccgccgccgccgccggcgaccacgagccggagttcatcggcagccctgttgccgcggacgaggcgcgcagcaactggcccaagcgctacggccgcagcactgccgcaaagaaaccggatgaggaggaagagctcaaggccagatgtcactaccggagcgctaaggtggacaacgtcgtctactgccttggggatgacgtctatgtcaaggctggagaaaacgaggcagattacattggccgcattactgaattttttgaggggactgaccagtgtcactattttacttgccgttggttcttccgagcagaggacacggttatcaattctttggtgtccataagtgtggatggccacaagcatgaccctagacgtgtttttctttctgaggaaaagaacgacaatgtgcttgattgcattatctccaaggtcaagatagtccatgttgatccaaatatggatccaaaagccaaggctcagctgatagagagttgcgacctatactatgacatgtcttactctgttgcatattctacatttgctaatatctcgtctgaaaatgggcagtcagacagtgataccgcttcgggtatttcttctgatgatgtggatctggagacgtcatctagtatgccaacgaggacagcaacccttcttgatctgtattctggctgtgggggcatgtctactggtctttgcttgggtgcagctctttctggcttgaaacttgaaactcgatgggctgttgatttcaacagttttgcgtgccaaagtttaaaatataatcatccacagactgaggtgcgaaatgagaaagccgatgagtttcttgccctccttaaggaatgggcagttctatgcaaaaaatatgtccaagatgtggattcaaatttagcaagctcagaggatcaagcggatgaagacagccctcttgacaaggacgaatttgttgtagagaagcttgtcgggatatgttatggtggcagtgacagggaaaatggcatctattttaaggtccagtgggaaggatacggccctgaggaggatacatgggaaccgattgataacttgagtgactgcccgcagaaaattagagattttgtacaagaagggcacaaaagaaagattctcccactgcctggtgatgttgatgtcatttgtggaggcccaccatgccaaggtatcagtgggtttaatcggtacagaaaccgtgatgagccactcaaagatgagaaaaacaaacaaatggtgactttcatggatattgtggcgtacttgaagcccaagtatgttctcatggaaaatgtggtggacatactcaaatttgcggatggttacctaggaaaatatgctttgagctgccttgttgctatgaagtaccaagcgcggcttggaatgatggtggctggttgctatggtctgccacagttcaggatgcgtgtgttcctctggggtgctctttcttccatggtgctccctaagtatcctctgcccacctatgatgttgtagtacgtggaggagcccctaatgccttttcgcaatgtatggttgcatatgacgagacacaaaaaccatccctgaaaaaagccttgcttcttggcgatgcaatttcagatttaccaaaggttcaaaatcaccagcctaacgatgtgatggagtatggtggttcccccaagaccgaattccagcgctacattcgactcagtcgtaaagacatgttggattggtccttcggtgagggggctggtccagatgaaggcaagctcttggatcaccagcctttacggcttaacaacgatgattatgagcgggttcaacagattcctgtcaagaagggagccaacttccgcgacctaaagggcgtgagggttggagcaaacaatattgttgagtgggatccagaaatcgagcgtgtgaaactttcatctgggaaaccactggttcctgactatgcaatgtcattcatcaagggcaaatcactcaagccgtttgggcgcctgtggtgggacgagacagttcctacagttgtaaccagagcagagcctcacaaccaggttataattcatccgactcaagcaagggtcctcactatccgggagaacgcaaggttacagggcttccccgattactaccgattgtttggcccgatcaaggagaagtacattcaagtcgggaacgcagtggctgtccctgttgcccgggcactgggctactgtctggggcaagcctacctgggtgaatctgaggggagtgaccctctgtaccagctgcctccaagtttcacctctgttggaggacgcactgcggggcaggcgagggcctctcctgttggcacccctgcaggggaggtagttgagcagtaa

SEQ ID NO: 9 - DNA, 11129 bp, Artificial sequence
  Nucleic acid
tacacaagaggagcgaagcaagtttcatccaacaggcttcatcacggtgaaacgggtatacaacgtggcttcggtataactcaaaggaagtataacgaagcacacttcatcaagcaggcttcgtcataacgaaacaatgagatgcaaggagaaggtgttactcaagttattaaatacaactgacgacattgcccttatgacatattgtaaactctgtatgtaattattgagggtataaatgtaatagcgtacgaacccttgcgattgtctataaatagatgaacattaccccgtactattcacgttggattgtaatcgctctcgcgtcaacaccttcaaacaagccgaagatatcactgcaatataaattttgataatgtttatatatttatcatggaataaatatatgactggatatcacactcatgtatttaaatccttattgatataaatgatgaggatgtgttattcacgacctttgtctgaagattattatatccgaaaggagatattttttcgaaggacgaatgttcttaacatttaataactgtgttaccttgttcttgaacatgtgacaaacattaggttttgagtatttcaattaatagtgaaattaaattaaattttagttgtccaaaatgacagacttcgatatttgttataattgttgagttaaattttaacagcatccataaacaagtaaacaacgactatgtgtgcaccagtgtctcctgtatcaacggggtggtccgagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttcgtctggaggaggtaccgaggtcccgccgttcgagagacgagctacgcatttaagccggggaggtcggagcagtgagtgggagtagttcagggactgggagaggagaggaagcaggacctgggagacctcgcctcgtccgtcctcctgcctacttccttgcttttggtaagtgaaagtgattgaagtggagatgcctccaatctgcagtgctgtttcacgcgtaaaccctggccccctcgccgctggaagccccattgttcctctttcatttcttaccgctgatgcagcttatggtttcctagggttttcgagatatttggtttcttgcatccagaggacgaagagctttcatagggttttcgagacattgtcctcacaatcgcatccattctgcagtgctgtttcacgcgtaaaccctagcccctcgtcgctggaagcccacatggttcctctttcatttcttaccgctgatgcagcttatgggttcctagggttttcgagacatttggcttcttgcatccagaggacgaagagctttcctagggttttcgagacattgtcctcacaatcgcagatttggttcgagacatttgtttnnnnnnnnnncgtcccgcgcttgagtccttgtgatctgcacagcatgatgctgccaccaaatgcatgcagttagacaagcttaatgttgcatctgataagactgtacaacagattcgtgtgccaaatgtgtccttgctactcaaggaggtcatatggaacaagtaaaacgaaatttctgcatcagcaatcaggcacaagactgaatgacgtggttagtgccaatgaggtggggacttgggaatggaattagagttatagctattcctataattcatctcactacagaggctctccatcctactaggactgtacaacagattcgtgtgccaaatgtgtccttgctactcaaggaggtcatatggaacaagtaaaacaaaatttctgcatcagcaatcaggcacaagactgaatgacgtggttagtgccaatgaggtgggaacttgggaatggaattagagttatagctattctatagctattcctataattcatctcactacagaggctctccatcctactagaatttaatctgataaagtaggcagaaacatggaacttataatgtcgaagattgcatgtatagaatctaagtaacggaaagattcaactttgtgattatatgctgctgttacatggtattggtaaaacattctttcaagtgctcattgtcttcccttgcaaccaattcattgttgtccttgtttccttgcaggtaggtgctgcttgttttatcttgaaatgggctctcatgatggcgaggatgaagagttgccacccccccctccggtgccaccagatgtgattcccattaaagctgaagatgctgtgggtgaatcaccagcaaaccatatattaaagccaaagagattactgatggacaggcctggtataggaagaaaagggcagccgacccagctctattcaaatcactttaaagtcgctgtgaagagtacagaagacgtcttctttcactactatgtatgtctgctgactgagttcatgatcctttgttcaaaatatgtcattgtctgtcccttgtttataactaatctttggctgatgtgatattgttattttatttgactaggtaaacctgaagtatgaggatgatcgacccgttgatggtaaagggatcggcagaaaggtgattgataaactgcagcagacatatcgtgcagagctttctaacaaggactttgtatatgatggagaaaagagcctgtttacagttggtggtcttccacaaaaaaagaatgagttcaccgttgtcttggaggacgtatctactggaaagttagttttagtctttgatctgctttcttgttgtttatgcttacccagaatcagtaattgccatgttttttgtttgtggttgtaggactgctgccaatgggagccctggaggtaatgacagtcctggaggtggtgataggaagagagtgaggaggccataccagacgaaaactttcaaagtggagataaattttgcagcagaggttcctatgagtgctattggtcaagtcattagaggcgaagaatctgagaactccctggaggcgcttcgtgttcttgatatcatactgaggcagcattccgcagaacagtatgtagctatgcatcttgatggatgattataggtagatttgaggatcttctgcagttacgacatgacaatttttctcaatacttgcagaggctgccttttggttaagcaatcatttttctacaacaacccttcatgctttgttgacttgggtggtggtgtgatgggttgtcgtggatttcattcaagcttccgtggcacacagagtggactttccctcaatgttggtgcgtgttcgtcctgtatggctgtattggtgccatgtaactgcattgtctattctataatcttacatctttcatgatcatgatagcctttttttttgaaatccacagatgtctcaacaacaatgatcgtgaaacctggccctgttattgattttcttctttctaaccagaatgttaatgatcctagcagaattgattggcaaaaggtaaatgccatctttggatgaaacttctcgaataaccatgctagtgcattgaaatatctattttgatagaatgataacacaattttggggtgtgcatattcaatacaggccaagcgtgctctcaagggcttgaggattagaaccactcctgcaaattcagaattcaagatttttggtctcagcgagaggatctgcaaagaacaaacgtgtggacatgttttcttaccgctctgtttattgtcatgcaagctttactttacatttttaaaaaattgtaccctagtgtgttaagcttgtttcagaagaattttatttaacaaatattgcctttattgattttgttctgattccaatgccaagcttgcttgatggtgatatctgtgtgcttaattgctttctcattgacatatgcaattaggtttccgctgaggcagagaaatggtagcaacggagattgtgataccattgaaataactgtctatgactactatgcaaagaaaggaatcgatctaaagtattctggtgatttcccctgtataaatacagggaaggcaaagcgcccaacatattttccaatcgaggtttgtttcagttttgttagttacatcctgtcaaatctctgtttattaaatatatcttgcatctcatattcgatgggcaggcttggtggggctgtctcactgagtcactaggtcgtgggttcaaagcagcctctccacatttgtgggggggggaaggcttgcttcggtttatcccttccttagaccctaggtctgcccctttttttgcatctcatttttttgtattgtgtttctagctatgctcgcttgttccgcttcaaagatacaccaaagctttgtctacgctacaaaggtcatcccttgtggagaagtctagacagaagcctgaagaaaggatgaccgttctaaatgatgtgagctgttaccttgattttagcatgctgcgaccattcataactgcatgggatttatttctacgactgaattaatcaacagcttatgattaccctaaatgctaggcactgcaacgcagtaactacgattctgaccccatgttgatgtgcnnnnnnnnnnaggttgagggcatgtggtgtttcagttgctccaaaatttacccaagttgaaggaaggatccttcaagccccaaaggtnnnnnnnnnngcttgnnagagaatcctagggtgtttggatggaattctaggatttaaagatttttttcctatcctgattgttaatatctctatggatccaaacgctcccttagttgttttctctctaaatgttgtaccaagtcattatctcagtaaaatgcatctgactaaatctaatatcttgtagctgaaagccggcaatggtgatgatatcttttcacgaaatggacggtggaatttcactaatagggtcagcagctcaacatgtttcttttttaaccttctttttttctttattaggaaggatttttctgatgtttatttgccaatactttgcccctttcagaagttttatgaaacctgctctgtgaataagtgggcggtcgttaatttctctgcacgttgtgatgttcggaatcttatccgtgacctgatgaggaatgcatctgcaaagggaattgtatggtttagcttttcacatatcttctattgtagcagttttctgctgctgacgttttcttctggcagcaaatggaggaaccttttgatgtgtttgaagagagtccctctatgaggcgtgcacctgtgtcaagaagggtggatgatatgtttgggcagataaaatcaaaacttcctggagctcctaggttcctcttgtgccttctccctgagaggaaaaattgtgaaatctatggttggtttattttttgttaccactccacataaatatctcacatcttgcaataattatacctttttggtttcaaccaggtccttggaagagaaagtgcctggccgagtttggtattgtcacacagtgtctagctccattaagagtcaatgatccgtacctgcttaatttgctgatgaagatcaatgcaaaggtttgggacatgattttatctttcttttgcactgtattgttccctggttcatgattatattatcctctttcctctgtttttaactagcttggtggtctgaactcgttgctgcaagttgaagcatcttcgtcaataccacatgtgtcgcaagtacccaccatcatcttaggtatggatgtttcacatggtcatccaggacaagatagaccttcggttgcagcggtaagatgggtgtacctttacttgtacaattcttctgcccacatggaatctgagtggttttgtaaaatatgtgaaatggatgtctgttaatctgatgtaaaccatacctttcttctttgtttaggtggttagttctcgtcaatggcctcttatctctagatatagagcatcagtgcacacccaatctgccagactagaaatgatgtcctcgttgtttaagccgcggggtactgatgatgatggcctcatccggtaagagatatcttcttagtatgttgatgtatggtaagcgtgatgacttttggatgctaagtttatcttcgtatttgggacttccagggaatcactgatcgacttctacactagctctggaaagcgaaaaccagaacacataattattttcaggtctgaagctttggggttattgttagcttgtatcactaaatctttttgtagaaacaaattcttttttttgtttttgttttgagaagtatgtatggtacattctttgctgggatggattattgatactttcttagccgccgtggcgtaaatgtaaccatttgctgggttttagggatggagtcagtgaaagtcagtttacccaggtcatcaacattgagctggatcagatcatcgaggtaatacatttgtttgattcgtcctttgcacatttgcttgcttagttcaggaacgaattgcaaggtaacacatccttgtaattaatgcaggcatgtaagtttctggatgagaagtggtcacccaagttcactgtgattgttgctcaaaagaaccaccacaccaagttctttcagacggcatcaccagacaatgttcttcctggtaatatgtttatttttgtcatactaaagggagtgtggtgagatgcggtgacaataataaatgtgtttgtttgttacgcaggaactgtggtggatagtaaagtttgccatcctaagaacttcgacttctacatgtgtgcacatgctgggatgattgtgagttgatctcagaattgagcaaaaagatgctgctgtcttgttcttgtctctgaatcctagatggttcttcgtctcagggaacaacaaggccgacccactatcatgttctgcacgacgagataggtttcagtgccgacgagatgcaggagtttgttcattcgctctcttacgtgtaagtagtagtagtaaaatgctgttttggctgtgtgtgtgtgtgtgtgttttgtgcttacgctgcgtgtcctgctgtgcaggtaccagaggagcacgacagccatctcagtgggtaggccgcttgaatctaaagccatcttgtgcagcacagggacagcaggttgatttttttctagtgtctgtttttgtgtttgcagttgctccagtgtgctacgcccacctcgctgcagcccaggtgagcacgttcctgagattggaggagatgtcagacgcgtcctccagccagggaggagggcatacctcggctggcagtgctcctgtgccggagctgcctcgcctgcatgacaaagtcaggagctccatgttcttctgctagctgatgtgcgtgcgcatcaggatcgagctccatgttttgtgttagtaaggcctagttagtaaggctgtagaaagaatgtttaatgtttgcatgctaaagtccaaacaatcaaaaccactactatatctaccagagcactgatcgatcaaacaacaagagtcagcatcaatcaatcaaaaccaccactattctatctaccgatcaatctactctatacctgaagcactcactcgctcgcaaacaaccaagggatcatggatccttgagtagctccattgtccagtccctgaccttgacgaagcatttcatggacagcctctcgtacgccatctcgtacatccggaggttcatgtaggcgaagctcaagtaggtggcgatgcactcttggtgcctgtggtacagcgccgggatggtcagagcggccaccacacttgcgtagcaggcggtcgggaaatctgtaacgctgccgacgatggacacgctccacaggcagaggaaggctctgtagaagagcagcgagtccctgcccagagcgatgtcccggaaagcgtccgaggcggcgtccagagcagagcgcagcagcgccgccgcctcgtccacggcctgctgcggaacgcgcagctccgggacgggaggctgcggcctgttcaggaggcgcgccgctttggcccacaggaagagcacggtgaggagcaggagcagcacgttggaggcgagggacaccaaggtgtagccgccgtacaagagcagccaggaggagacggtgcccgccagcaggcaggcgctcacgtcggcgcggccccggcgccacagcagcacgtcccagactgctgcagcaatccgatccgattccgatccagatatattagttagtagatccaatctcgagcggccaatccgtaataataattggctgagagaaaaggtgggagagagtaccgaatcctccgacggcacatgtcccggcgccgccgccgccgccgccaacaccaacaccagcatccaccaccatcgtcgtagttaatactaagggaagggaactagagaaggaagggaggggccgatgcaacggaagaaatggatggaattcgaccaacagctagctagagcgaagcagaacccaatcccaatcaaaacccgagaacgaacctgcttttactttcccaaccagacggtggctggcttggaatcccactatccctcattccctctaccaacagggcaccggcctggcctccctccccaatcatccaaccaccacctttttttttcttccttttgctgtgttttcttacaaacttaaatctactcgaatattgattcaagatattatggagagtaattgaatatttcgatgaatcacgtcatgtatatatttcttaattacaaacttctatcactatcaaataaagtaaacataatgctatttgcaataatatatactatgtaaacatgactaaatcaaaataaatgtttaatttaatagttaccaagtttgcaagaaaacatttccttccaaagcacatatcatgatgctggcaatttagttttatcagctataataatatcattgacaacacattttgtaagtccacattctaaataaattctgtacattataactaaaacattatatcatatcagcataaaaaataaattaagtattctatgtttctataaaacaataaaccatgcattgtacggtgactgcgtctagggaaaattataccgcatgctagcaaaacgtcctccagtatttcattagacacattggtgtcaacctaatatcctatttgttgtcgttccaactaaccccctcttatagtgctacaacactatcatctatgtgtgatggttttactcgtgatacctcacttacaggtgaaaataagaccctcaaaaaggaggcgaatgagctcattcacgtcttaattaaagcctacagtagtgaggctcacttgcttaaatgcttggatagtcaaagattctaggtattagactaagtaactattctgttttgctacaaatgtattagactaatgctgaagggcaaatgccgaaggtaggtttcgaccagggcgcgcaccaagactttggtgatcattgtgaacatgcttcggacgaagctaattccgaaggtcctatgggcgacgacaaactaag

SEQ ID NO: 10 - DNA, 11129 bp, Artificial sequence
  Nucleic acid
tacacaagaggagcgaagcaagtttcatccaacaggcttcatcacggtgaaacgggtatacaacgtggcttcggtataactcaaaggaagtataacgaagcacacttcatcaagcaggcttcgtcataacgaaacaatgagatgcaaggagaaggtgttactcaagttattaaatacaactgacgacattgcccttatgacatattgtaaactctgtatgtaattattgagggtataaatgtaatagcgtacgaacccttgcgattgtctataaatagatgaacattaccccgtactattcacgttggattgtaatcgctctcgcgtcaacaccttcaaacaagccgaagatatcactgcaatataaattttgataatgtttatatatttatcatggaataaatatatgactggatatcacactcatgtatttaaatccttattgatataaatgatgaggatgtgttattcacgacctttgtctgaagattattatatccgaaaggagatattttttcgaaggacgaatgttcttaacatttaataactgtgttaccttgttcttgaacatgtgacaaacattaggttttgagtatttcaattaatagtgaaattaaattaaattttagttgtccaaaatgacagacttcgatatttgttataattgttgagttaaattttaacagcatccataaacaagtaaacaacgactatgtgtgcaccagtgtctcctgtatcaacggggtggtccgtctggaggaggtaccgagagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttcggtcccgccgttcgagagacgagctacgcatttaagccggggaggtcggagcagtgagtgggagtagttcagggactgggagaggagaggaagcaggacctgggagacctcgcctcgtccgtcctcctgcctacttccttgcttttggtaagtgaaagtgattgaagtggagatgcctccaatctgcagtgctgtttcacgcgtaaaccctggccccctcgccgctggaagccccattgttcctctttcatttcttaccgctgatgcagcttatggtttcctagggttttcgagatatttggtttcttgcatccagaggacgaagagctttcatagggttttcgagacattgtcctcacaatcgcatccattctgcagtgctgtttcacgcgtaaaccctagcccctcgtcgctggaagcccacatggttcctctttcatttcttaccgctgatgcagcttatgggttcctagggttttcgagacatttggcttcttgcatccagaggacgaagagctttcctagggttttcgagacattgtcctcacaatcgcagatttggttcgagacatttgtttnnnnnnnnnncgtcccgcgcttgagtccttgtgatctgcacagcatgatgctgccaccaaatgcatgcagttagacaagcttaatgttgcatctgataagactgtacaacagattcgtgtgccaaatgtgtccttgctactcaaggaggtcatatggaacaagtaaaacgaaatttctgcatcagcaatcaggcacaagactgaatgacgtggttagtgccaatgaggtggggacttgggaatggaattagagttatagctattcctataattcatctcactacagaggctctccatcctactaggactgtacaacagattcgtgtgccaaatgtgtccttgctactcaaggaggtcatatggaacaagtaaaacaaaatttctgcatcagcaatcaggcacaagactgaatgacgtggttagtgccaatgaggtgggaacttgggaatggaattagagttatagctattctatagctattcctataattcatctcactacagaggctctccatcctactagaatttaatctgataaagtaggcagaaacatggaacttataatgtcgaagattgcatgtatagaatctaagtaacggaaagattcaactttgtgattatatgctgctgttacatggtattggtaaaacattctttcaagtgctcattgtcttcccttgcaaccaattcattgttgtccttgtttccttgcaggtaggtgctgcttgttttatcttgaaatgggctctcatgatggcgaggatgaagagttgccacccccccctccggtgccaccagatgtgattcccattaaagctgaagatgctgtgggtgaatcaccagcaaaccatatattaaagccaaagagattactgatggacaggcctggtataggaagaaaagggcagccgacccagctctattcaaatcactttaaagtcgctgtgaagagtacagaagacgtcttctttcactactatgtatgtctgctgactgagttcatgatcctttgttcaaaatatgtcattgtctgtcccttgtttataactaatctttggctgatgtgatattgttattttatttgactaggtaaacctgaagtatgaggatgatcgacccgttgatggtaaagggatcggcagaaaggtgattgataaactgcagcagacatatcgtgcagagctttctaacaaggactttgtatatgatggagaaaagagcctgtttacagttggtggtcttccacaaaaaaagaatgagttcaccgttgtcttggaggacgtatctactggaaagttagttttagtctttgatctgctttcttgttgtttatgcttacccagaatcagtaattgccatgttttttgtttgtggttgtaggactgctgccaatgggagccctggaggtaatgacagtcctggaggtggtgataggaagagagtgaggaggccataccagacgaaaactttcaaagtggagataaattttgcagcagaggttcctatgagtgctattggtcaagtcattagaggcgaagaatctgagaactccctggaggcgcttcgtgttcttgatatcatactgaggcagcattccgcagaacagtatgtagctatgcatcttgatggatgattataggtagatttgaggatcttctgcagttacgacatgacaatttttctcaatacttgcagaggctgccttttggttaagcaatcatttttctacaacaacccttcatgctttgttgacttgggtggtggtgtgatgggttgtcgtggatttcattcaagcttccgtggcacacagagtggactttccctcaatgttggtgcgtgttcgtcctgtatggctgtattggtgccatgtaactgcattgtctattctataatcttacatctttcatgatcatgatagcctttttttttgaaatccacagatgtctcaacaacaatgatcgtgaaacctggccctgttattgattttcttctttctaaccagaatgttaatgatcctagcagaattgattggcaaaaggtaaatgccatctttggatgaaacttctcgaataaccatgctagtgcattgaaatatctattttgatagaatgataacacaattttggggtgtgcatattcaatacaggccaagcgtgctctcaagggcttgaggattagaaccactcctgcaaattcagaattcaagatttttggtctcagcgagaggatctgcaaagaacaaacgtgtggacatgttttcttaccgctctgtttattgtcatgcaagctttactttacatttttaaaaaattgtaccctagtgtgttaagcttgtttcagaagaattttatttaacaaatattgcctttattgattttgttctgattccaatgccaagcttgcttgatggtgatatctgtgtgcttaattgctttctcattgacatatgcaattaggtttccgctgaggcagagaaatggtagcaacggagattgtgataccattgaaataactgtctatgactactatgcaaagaaaggaatcgatctaaagtattctggtgatttcccctgtataaatacagggaaggcaaagcgcccaacatattttccaatcgaggtttgtttcagttttgttagttacatcctgtcaaatctctgtttattaaatatatcttgcatctcatattcgatgggcaggcttggtggggctgtctcactgagtcactaggtcgtgggttcaaagcagcctctccacatttgtgggggggggaaggcttgcttcggtttatcccttccttagaccctaggtctgcccctttttttgcatctcatttttttgtattgtgtttctagctatgctcgcttgttccgcttcaaagatacaccaaagctttgtctacgctacaaaggtcatcccttgtggagaagtctagacagaagcctgaagaaaggatgaccgttctaaatgatgtgagctgttaccttgattttagcatgctgcgaccattcataactgcatgggatttatttctacgactgaattaatcaacagcttatgattaccctaaatgctaggcactgcaacgcagtaactacgattctgaccccatgttgatgtgcnnnnnnnnnnaggttgagggcatgtggtgtttcagttgctccaaaatttacccaagttgaaggaaggatccttcaagccccaaaggtnnnnnnnnnngcttgnnagagaatcctagggtgtttggatggaattctaggatttaaagatttttttcctatcctgattgttaatatctctatggatccaaacgctcccttagttgttttctctctaaatgttgtaccaagtcattatctcagtaaaatgcatctgactaaatctaatatcttgtagctgaaagccggcaatggtgatgatatcttttcacgaaatggacggtggaatttcactaatagggtcagcagctcaacatgtttcttttttaaccttctttttttctttattaggaaggatttttctgatgtttatttgccaatactttgcccctttcagaagttttatgaaacctgctctgtgaataagtgggcggtcgttaatttctctgcacgttgtgatgttcggaatcttatccgtgacctgatgaggaatgcatctgcaaagggaattgtatggtttagcttttcacatatcttctattgtagcagttttctgctgctgacgttttcttctggcagcaaatggaggaaccttttgatgtgtttgaagagagtccctctatgaggcgtgcacctgtgtcaagaagggtggatgatatgtttgggcagataaaatcaaaacttcctggagctcctaggttcctcttgtgccttctccctgagaggaaaaattgtgaaatctatggttggtttattttttgttaccactccacataaatatctcacatcttgcaataattatacctttttggtttcaaccaggtccttggaagagaaagtgcctggccgagtttggtattgtcacacagtgtctagctccattaagagtcaatgatccgtacctgcttaatttgctgatgaagatcaatgcaaaggtttgggacatgattttatctttcttttgcactgtattgttccctggttcatgattatattatcctctttcctctgtttttaactagcttggtggtctgaactcgttgctgcaagttgaagcatcttcgtcaataccacatgtgtcgcaagtacccaccatcatcttaggtatggatgtttcacatggtcatccaggacaagatagaccttcggttgcagcggtaagatgggtgtacctttacttgtacaattcttctgcccacatggaatctgagtggttttgtaaaatatgtgaaatggatgtctgttaatctgatgtaaaccatacctttcttctttgtttaggtggttagttctcgtcaatggcctcttatctctagatatagagcatcagtgcacacccaatctgccagactagaaatgatgtcctcgttgtttaagccgcggggtactgatgatgatggcctcatccggtaagagatatcttcttagtatgttgatgtatggtaagcgtgatgacttttggatgctaagtttatcttcgtatttgggacttccagggaatcactgatcgacttctacactagctctggaaagcgaaaaccagaacacataattattttcaggtctgaagctttggggttattgttagcttgtatcactaaatctttttgtagaaacaaattcttttttttgtttttgttttgagaagtatgtatggtacattctttgctgggatggattattgatactttcttagccgccgtggcgtaaatgtaaccatttgctgggttttagggatggagtcagtgaaagtcagtttacccaggtcatcaacattgagctggatcagatcatcgaggtaatacatttgtttgattcgtcctttgcacatttgcttgcttagttcaggaacgaattgcaaggtaacacatccttgtaattaatgcaggcatgtaagtttctggatgagaagtggtcacccaagttcactgtgattgttgctcaaaagaaccaccacaccaagttctttcagacggcatcaccagacaatgttcttcctggtaatatgtttatttttgtcatactaaagggagtgtggtgagatgcggtgacaataataaatgtgtttgtttgttacgcaggaactgtggtggatagtaaagtttgccatcctaagaacttcgacttctacatgtgtgcacatgctgggatgattgtgagttgatctcagaattgagcaaaaagatgctgctgtcttgttcttgtctctgaatcctagatggttcttcgtctcagggaacaacaaggccgacccactatcatgttctgcacgacgagataggtttcagtgccgacgagatgcaggagtttgttcattcgctctcttacgtgtaagtagtagtagtaaaatgctgttttggctgtgtgtgtgtgtgtgtgttttgtgcttacgctgcgtgtcctgctgtgcaggtaccagaggagcacgacagccatctcagtgggtaggccgcttgaatctaaagccatcttgtgcagcacagggacagcaggttgatttttttctagtgtctgtttttgtgtttgcagttgctccagtgtgctacgcccacctcgctgcagcccaggtgagcacgttcctgagattggaggagatgtcagacgcgtcctccagccagggaggagggcatacctcggctggcagtgctcctgtgccggagctgcctcgcctgcatgacaaagtcaggagctccatgttcttctgctagctgatgtgcgtgcgcatcaggatcgagctccatgttttgtgttagtaaggcctagttagtaaggctgtagaaagaatgtttaatgtttgcatgctaaagtccaaacaatcaaaaccactactatatctaccagagcactgatcgatcaaacaacaagagtcagcatcaatcaatcaaaaccaccactattctatctaccgatcaatctactctatacctgaagcactcactcgctcgcaaacaaccaagggatcatggatccttgagtagctccattgtccagtccctgaccttgacgaagcatttcatggacagcctctcgtacgccatctcgtacatccggaggttcatgtaggcgaagctcaagtaggtggcgatgcactcttggtgcctgtggtacagcgccgggatggtcagagcggccaccacacttgcgtagcaggcggtcgggaaatctgtaacgctgccgacgatggacacgctccacaggcagaggaaggctctgtagaagagcagcgagtccctgcccagagcgatgtcccggaaagcgtccgaggcggcgtccagagcagagcgcagcagcgccgccgcctcgtccacggcctgctgcggaacgcgcagctccgggacgggaggctgcggcctgttcaggaggcgcgccgctttggcccacaggaagagcacggtgaggagcaggagcagcacgttggaggcgagggacaccaaggtgtagccgccgtacaagagcagccaggaggagacggtgcccgccagcaggcaggcgctcacgtcggcgcggccccggcgccacagcagcacgtcccagactgctgcagcaatccgatccgattccgatccagatatattagttagtagatccaatctcgagcggccaatccgtaataataattggctgagagaaaaggtgggagagagtaccgaatcctccgacggcacatgtcccggcgccgccgccgccgccgccaacaccaacaccagcatccaccaccatcgtcgtagttaatactaagggaagggaactagagaaggaagggaggggccgatgcaacggaagaaatggatggaattcgaccaacagctagctagagcgaagcagaacccaatcccaatcaaaacccgagaacgaacctgcttttactttcccaaccagacggtggctggcttggaatcccactatccctcattccctctaccaacagggcaccggcctggcctccctccccaatcatccaaccaccacctttttttttcttccttttgctgtgttttcttacaaacttaaatctactcgaatattgattcaagatattatggagagtaattgaatatttcgatgaatcacgtcatgtatatatttcttaattacaaacttctatcactatcaaataaagtaaacataatgctatttgcaataatatatactatgtaaacatgactaaatcaaaataaatgtttaatttaatagttaccaagtttgcaagaaaacatttccttccaaagcacatatcatgatgctggcaatttagttttatcagctataataatatcattgacaacacattttgtaagtccacattctaaataaattctgtacattataactaaaacattatatcatatcagcataaaaaataaattaagtattctatgtttctataaaacaataaaccatgcattgtacggtgactgcgtctagggaaaattataccgcatgctagcaaaacgtcctccagtatttcattagacacattggtgtcaacctaatatcctatttgttgtcgttccaactaaccccctcttatagtgctacaacactatcatctatgtgtgatggttttactcgtgatacctcacttacaggtgaaaataagaccctcaaaaaggaggcgaatgagctcattcacgtcttaattaaagcctacagtagtgaggctcacttgcttaaatgcttggatagtcaaagattctaggtattagactaagtaactattctgttttgctacaaatgtattagactaatgctgaagggcaaatgccgaaggtaggtttcgaccagggcgcgcaccaagactttggtgatcattgtgaacatgcttcggacgaagctaattccgaaggtcctatgggcgacgacaaactaag

SEQ ID NO: 11 - DNA, 11129 bp, Artificial sequence
  Nucleic acid
tacacaagaggagcgaagcaagtttcatccaacaggcttcatcacggtgaaacgggtatacaacgtggcttcggtataactcaaaggaagtataacgaagcacacttcatcaagcaggcttcgtcataacgaaacaatgagatgcaaggagaaggtgttactcaagttattaaatacaactgacgacattgcccttatgacatattgtaaactctgtatgtaattattgagggtataaatgtaatagcgtacgaacccttgcgattgtctataaatagatgaacattaccccgtactattcacgttggattgtaatcgctctcgcgtcaacaccttcaaacaagccgaagatatcactgcaatataaattttgataatgtttatatatttatcatggaataaatatatgactggatatcacactcatgtatttaaatccttattgatataaatgatgaggatgtgttattcacgacctttgtctgaagattattatatccgaaaggagatattttttcgaaggacgaatgttcttaacatttaataactgtgttaccttgttcttgaacatgtgacaaacattaggttttgagtatttcaattaatagtgaaattaaattaaattttagttgtccaaaatgacagacttcgatatttgttataattgttgagttaaattttaacagcatccataaacaagtaaacaacgactatgtgtgcaccagtgtctcctgtatcaacggggtggtccgtctggaggaggtaccgaggtccgagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttccgccgttcgagagacgagctacgcatttaagccggggaggtcggagcagtgagtgggagtagttcagggactgggagaggagaggaagcaggacctgggagacctcgcctcgtccgtcctcctgcctacttccttgcttttggtaagtgaaagtgattgaagtggagatgcctccaatctgcagtgctgtttcacgcgtaaaccctggccccctcgccgctggaagccccattgttcctctttcatttcttaccgctgatgcagcttatggtttcctagggttttcgagatatttggtttcttgcatccagaggacgaagagctttcatagggttttcgagacattgtcctcacaatcgcatccattctgcagtgctgtttcacgcgtaaaccctagcccctcgtcgctggaagcccacatggttcctctttcatttcttaccgctgatgcagcttatgggttcctagggttttcgagacatttggcttcttgcatccagaggacgaagagctttcctagggttttcgagacattgtcctcacaatcgcagatttggttcgagacatttgtttnnnnnnnnnncgtcccgcgcttgagtccttgtgatctgcacagcatgatgctgccaccaaatgcatgcagttagacaagcttaatgttgcatctgataagactgtacaacagattcgtgtgccaaatgtgtccttgctactcaaggaggtcatatggaacaagtaaaacgaaatttctgcatcagcaatcaggcacaagactgaatgacgtggttagtgccaatgaggtggggacttgggaatggaattagagttatagctattcctataattcatctcactacagaggctctccatcctactaggactgtacaacagattcgtgtgccaaatgtgtccttgctactcaaggaggtcatatggaacaagtaaaacaaaatttctgcatcagcaatcaggcacaagactgaatgacgtggttagtgccaatgaggtgggaacttgggaatggaattagagttatagctattctatagctattcctataattcatctcactacagaggctctccatcctactagaatttaatctgataaagtaggcagaaacatggaacttataatgtcgaagattgcatgtatagaatctaagtaacggaaagattcaactttgtgattatatgctgctgttacatggtattggtaaaacattctttcaagtgctcattgtcttcccttgcaaccaattcattgttgtccttgtttccttgcaggtaggtgctgcttgttttatcttgaaatgggctctcatgatggcgaggatgaagagttgccacccccccctccggtgccaccagatgtgattcccattaaagctgaagatgctgtgggtgaatcaccagcaaaccatatattaaagccaaagagattactgatggacaggcctggtataggaagaaaagggcagccgacccagctctattcaaatcactttaaagtcgctgtgaagagtacagaagacgtcttctttcactactatgtatgtctgctgactgagttcatgatcctttgttcaaaatatgtcattgtctgtcccttgtttataactaatctttggctgatgtgatattgttattttatttgactaggtaaacctgaagtatgaggatgatcgacccgttgatggtaaagggatcggcagaaaggtgattgataaactgcagcagacatatcgtgcagagctttctaacaaggactttgtatatgatggagaaaagagcctgtttacagttggtggtcttccacaaaaaaagaatgagttcaccgttgtcttggaggacgtatctactggaaagttagttttagtctttgatctgctttcttgttgtttatgcttacccagaatcagtaattgccatgttttttgtttgtggttgtaggactgctgccaatgggagccctggaggtaatgacagtcctggaggtggtgataggaagagagtgaggaggccataccagacgaaaactttcaaagtggagataaattttgcagcagaggttcctatgagtgctattggtcaagtcattagaggcgaagaatctgagaactccctggaggcgcttcgtgttcttgatatcatactgaggcagcattccgcagaacagtatgtagctatgcatcttgatggatgattataggtagatttgaggatcttctgcagttacgacatgacaatttttctcaatacttgcagaggctgccttttggttaagcaatcatttttctacaacaacccttcatgctttgttgacttgggtggtggtgtgatgggttgtcgtggatttcattcaagcttccgtggcacacagagtggactttccctcaatgttggtgcgtgttcgtcctgtatggctgtattggtgccatgtaactgcattgtctattctataatcttacatctttcatgatcatgatagcctttttttttgaaatccacagatgtctcaacaacaatgatcgtgaaacctggccctgttattgattttcttctttctaaccagaatgttaatgatcctagcagaattgattggcaaaaggtaaatgccatctttggatgaaacttctcgaataaccatgctagtgcattgaaatatctattttgatagaatgataacacaattttggggtgtgcatattcaatacaggccaagcgtgctctcaagggcttgaggattagaaccactcctgcaaattcagaattcaagatttttggtctcagcgagaggatctgcaaagaacaaacgtgtggacatgttttcttaccgctctgtttattgtcatgcaagctttactttacatttttaaaaaattgtaccctagtgtgttaagcttgtttcagaagaattttatttaacaaatattgcctttattgattttgttctgattccaatgccaagcttgcttgatggtgatatctgtgtgcttaattgctttctcattgacatatgcaattaggtttccgctgaggcagagaaatggtagcaacggagattgtgataccattgaaataactgtctatgactactatgcaaagaaaggaatcgatctaaagtattctggtgatttcccctgtataaatacagggaaggcaaagcgcccaacatattttccaatcgaggtttgtttcagttttgttagttacatcctgtcaaatctctgtttattaaatatatcttgcatctcatattcgatgggcaggcttggtggggctgtctcactgagtcactaggtcgtgggttcaaagcagcctctccacatttgtgggggggggaaggcttgcttcggtttatcccttccttagaccctaggtctgcccctttttttgcatctcatttttttgtattgtgtttctagctatgctcgcttgttccgcttcaaagatacaccaaagctttgtctacgctacaaaggtcatcccttgtggagaagtctagacagaagcctgaagaaaggatgaccgttctaaatgatgtgagctgttaccttgattttagcatgctgcgaccattcataactgcatgggatttatttctacgactgaattaatcaacagcttatgattaccctaaatgctaggcactgcaacgcagtaactacgattctgaccccatgttgatgtgcnnnnnnnnnnaggttgagggcatgtggtgtttcagttgctccaaaatttacccaagttgaaggaaggatccttcaagccccaaaggtnnnnnnnnnngcttgnnagagaatcctagggtgtttggatggaattctaggatttaaagatttttttcctatcctgattgttaatatctctatggatccaaacgctcccttagttgttttctctctaaatgttgtaccaagtcattatctcagtaaaatgcatctgactaaatctaatatcttgtagctgaaagccggcaatggtgatgatatcttttcacgaaatggacggtggaatttcactaatagggtcagcagctcaacatgtttcttttttaaccttctttttttctttattaggaaggatttttctgatgtttatttgccaatactttgcccctttcagaagttttatgaaacctgctctgtgaataagtgggcggtcgttaatttctctgcacgttgtgatgttcggaatcttatccgtgacctgatgaggaatgcatctgcaaagggaattgtatggtttagcttttcacatatcttctattgtagcagttttctgctgctgacgttttcttctggcagcaaatggaggaaccttttgatgtgtttgaagagagtccctctatgaggcgtgcacctgtgtcaagaagggtggatgatatgtttgggcagataaaatcaaaacttcctggagctcctaggttcctcttgtgccttctccctgagaggaaaaattgtgaaatctatggttggtttattttttgttaccactccacataaatatctcacatcttgcaataattatacctttttggtttcaaccaggtccttggaagagaaagtgcctggccgagtttggtattgtcacacagtgtctagctccattaagagtcaatgatccgtacctgcttaatttgctgatgaagatcaatgcaaaggtttgggacatgattttatctttcttttgcactgtattgttccctggttcatgattatattatcctctttcctctgtttttaactagcttggtggtctgaactcgttgctgcaagttgaagcatcttcgtcaataccacatgtgtcgcaagtacccaccatcatcttaggtatggatgtttcacatggtcatccaggacaagatagaccttcggttgcagcggtaagatgggtgtacctttacttgtacaattcttctgcccacatggaatctgagtggttttgtaaaatatgtgaaatggatgtctgttaatctgatgtaaaccatacctttcttctttgtttaggtggttagttctcgtcaatggcctcttatctctagatatagagcatcagtgcacacccaatctgccagactagaaatgatgtcctcgttgtttaagccgcggggtactgatgatgatggcctcatccggtaagagatatcttcttagtatgttgatgtatggtaagcgtgatgacttttggatgctaagtttatcttcgtatttgggacttccagggaatcactgatcgacttctacactagctctggaaagcgaaaaccagaacacataattattttcaggtctgaagctttggggttattgttagcttgtatcactaaatctttttgtagaaacaaattcttttttttgtttttgttttgagaagtatgtatggtacattctttgctgggatggattattgatactttcttagccgccgtggcgtaaatgtaaccatttgctgggttttagggatggagtcagtgaaagtcagtttacccaggtcatcaacattgagctggatcagatcatcgaggtaatacatttgtttgattcgtcctttgcacatttgcttgcttagttcaggaacgaattgcaaggtaacacatccttgtaattaatgcaggcatgtaagtttctggatgagaagtggtcacccaagttcactgtgattgttgctcaaaagaaccaccacaccaagttctttcagacggcatcaccagacaatgttcttcctggtaatatgtttatttttgtcatactaaagggagtgtggtgagatgcggtgacaataataaatgtgtttgtttgttacgcaggaactgtggtggatagtaaagtttgccatcctaagaacttcgacttctacatgtgtgcacatgctgggatgattgtgagttgatctcagaattgagcaaaaagatgctgctgtcttgttcttgtctctgaatcctagatggttcttcgtctcagggaacaacaaggccgacccactatcatgttctgcacgacgagataggtttcagtgccgacgagatgcaggagtttgttcattcgctctcttacgtgtaagtagtagtagtaaaatgctgttttggctgtgtgtgtgtgtgtgtgttttgtgcttacgctgcgtgtcctgctgtgcaggtaccagaggagcacgacagccatctcagtgggtaggccgcttgaatctaaagccatcttgtgcagcacagggacagcaggttgatttttttctagtgtctgtttttgtgtttgcagttgctccagtgtgctacgcccacctcgctgcagcccaggtgagcacgttcctgagattggaggagatgtcagacgcgtcctccagccagggaggagggcatacctcggctggcagtgctcctgtgccggagctgcctcgcctgcatgacaaagtcaggagctccatgttcttctgctagctgatgtgcgtgcgcatcaggatcgagctccatgttttgtgttagtaaggcctagttagtaaggctgtagaaagaatgtttaatgtttgcatgctaaagtccaaacaatcaaaaccactactatatctaccagagcactgatcgatcaaacaacaagagtcagcatcaatcaatcaaaaccaccactattctatctaccgatcaatctactctatacctgaagcactcactcgctcgcaaacaaccaagggatcatggatccttgagtagctccattgtccagtccctgaccttgacgaagcatttcatggacagcctctcgtacgccatctcgtacatccggaggttcatgtaggcgaagctcaagtaggtggcgatgcactcttggtgcctgtggtacagcgccgggatggtcagagcggccaccacacttgcgtagcaggcggtcgggaaatctgtaacgctgccgacgatggacacgctccacaggcagaggaaggctctgtagaagagcagcgagtccctgcccagagcgatgtcccggaaagcgtccgaggcggcgtccagagcagagcgcagcagcgccgccgcctcgtccacggcctgctgcggaacgcgcagctccgggacgggaggctgcggcctgttcaggaggcgcgccgctttggcccacaggaagagcacggtgaggagcaggagcagcacgttggaggcgagggacaccaaggtgtagccgccgtacaagagcagccaggaggagacggtgcccgccagcaggcaggcgctcacgtcggcgcggccccggcgccacagcagcacgtcccagactgctgcagcaatccgatccgattccgatccagatatattagttagtagatccaatctcgagcggccaatccgtaataataattggctgagagaaaaggtgggagagagtaccgaatcctccgacggcacatgtcccggcgccgccgccgccgccgccaacaccaacaccagcatccaccaccatcgtcgtagttaatactaagggaagggaactagagaaggaagggaggggccgatgcaacggaagaaatggatggaattcgaccaacagctagctagagcgaagcagaacccaatcccaatcaaaacccgagaacgaacctgcttttactttcccaaccagacggtggctggcttggaatcccactatccctcattccctctaccaacagggcaccggcctggcctccctccccaatcatccaaccaccacctttttttttcttccttttgctgtgttttcttacaaacttaaatctactcgaatattgattcaagatattatggagagtaattgaatatttcgatgaatcacgtcatgtatatatttcttaattacaaacttctatcactatcaaataaagtaaacataatgctatttgcaataatatatactatgtaaacatgactaaatcaaaataaatgtttaatttaatagttaccaagtttgcaagaaaacatttccttccaaagcacatatcatgatgctggcaatttagttttatcagctataataatatcattgacaacacattttgtaagtccacattctaaataaattctgtacattataactaaaacattatatcatatcagcataaaaaataaattaagtattctatgtttctataaaacaataaaccatgcattgtacggtgactgcgtctagggaaaattataccgcatgctagcaaaacgtcctccagtatttcattagacacattggtgtcaacctaatatcctatttgttgtcgttccaactaaccccctcttatagtgctacaacactatcatctatgtgtgatggttttactcgtgatacctcacttacaggtgaaaataagaccctcaaaaaggaggcgaatgagctcattcacgtcttaattaaagcctacagtagtgaggctcacttgcttaaatgcttggatagtcaaagattctaggtattagactaagtaactattctgttttgctacaaatgtattagactaatgctgaagggcaaatgccgaaggtaggtttcgaccagggcgcgcaccaagactttggtgatcattgtgaacatgcttcggacgaagctaattccgaaggtcctatgggcgacgacaaactaag

SEQ ID NO: 12 - DNA, 11120 bp, Artificial sequence
  Nucleic acid
tacacaagaggagcgaagcaagtttcatccaacaggcttcatcacggtgaaacgggtatacaacgtggcttcggtataactcaaaggaagtataacgaagcacacttcatcaagcaggcttcgtcataacgaaacaatgagatgcaaggagaaggtgttactcaagttattaaatacaactgacgacattgcccttatgacatattgtaaactctgtatgtaattattgagggtataaatgtaatagcgtacgaacccttgcgattgtctataaatagatgaacattaccccgtactattcacgttggattgtaatcgctctcgcgtcaacaccttcaaacaagccgaagatatcactgcaatataaattttgataatgtttatatatttatcatggaataaatatatgactggatatcacactcatgtatttaaatccttattgatataaatgatgaggatgtgttattcacgacctttgtctgaagattattatatccgaaaggagatattttttcgaaggacgaatgttcttaacatttaataactgtgttaccttgttcttgaacatgtgacaaacattaggttttgagtatttcaattaatagtgaaattaaattaaattttagttgtccaaaatgacagacttcgatatttgttataattgttgagttaaattttaacagcatccataaacaagtaaacaacgactatgtgtgcaccagtgtctcctgtatcaacggggtggtccgtctggaggaggtaccgaggtcccgccgttcgagagacgagctacgcatttaagccggggaggtcggagcagtgagtgggagtagttcagggactgggagaggagaggaagcaggacctgggagacctcgcctcgtccgtcctcctgcctacttccttgcttttggtaagtgaaagtgattgaagtggagatgcctccaatctgcagtgctgtttcacgcgtaaaccctggccccctcgccgctggaagccccattgttcctctttcatttcttaccgctgatgcagcttatggtttcctagggttttcgagatatttggtttcttgcatccagaggacgaagagctttcatagggttttcgagacattgtcctcacaatcgcatccattctgcagtgctgtttcacgcgtaaaccctagcccctcgtcgctggaagcccacatggttcctctttcatttcttaccgctgatgcagcttatgggttcctagggttttcgagacatttggcttcttgcatccagaggacgaagagctttcctagggttttcgagacattgtcctcacaatcgcagatttggttcgagacatttgtttgagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttcncgtcccgcgcttgagtccttgtgatctgcacagcatgatgctgccaccaaatgcatgcagttagacaagcttaatgttgcatctgataagactgtacaacagattcgtgtgccaaatgtgtccttgctactcaaggaggtcatatggaacaagtaaaacgaaatttctgcatcagcaatcaggcacaagactgaatgacgtggttagtgccaatgaggtggggacttgggaatggaattagagttatagctattcctataattcatctcactacagaggctctccatcctactaggactgtacaacagattcgtgtgccaaatgtgtccttgctactcaaggaggtcatatggaacaagtaaaacaaaatttctgcatcagcaatcaggcacaagactgaatgacgtggttagtgccaatgaggtgggaacttgggaatggaattagagttatagctattctatagctattcctataattcatctcactacagaggctctccatcctactagaatttaatctgataaagtaggcagaaacatggaacttataatgtcgaagattgcatgtatagaatctaagtaacggaaagattcaactttgtgattatatgctgctgttacatggtattggtaaaacattctttcaagtgctcattgtcttcccttgcaaccaattcattgttgtccttgtttccttgcaggtaggtgctgcttgttttatcttgaaatgggctctcatgatggcgaggatgaagagttgccacccccccctccggtgccaccagatgtgattcccattaaagctgaagatgctgtgggtgaatcaccagcaaaccatatattaaagccaaagagattactgatggacaggcctggtataggaagaaaagggcagccgacccagctctattcaaatcactttaaagtcgctgtgaagagtacagaagacgtcttctttcactactatgtatgtctgctgactgagttcatgatcctttgttcaaaatatgtcattgtctgtcccttgtttataactaatctttggctgatgtgatattgttattttatttgactaggtaaacctgaagtatgaggatgatcgacccgttgatggtaaagggatcggcagaaaggtgattgataaactgcagcagacatatcgtgcagagctttctaacaaggactttgtatatgatggagaaaagagcctgtttacagttggtggtcttccacaaaaaaagaatgagttcaccgttgtcttggaggacgtatctactggaaagttagttttagtctttgatctgctttcttgttgtttatgcttacccagaatcagtaattgccatgttttttgtttgtggttgtaggactgctgccaatgggagccctggaggtaatgacagtcctggaggtggtgataggaagagagtgaggaggccataccagacgaaaactttcaaagtggagataaattttgcagcagaggttcctatgagtgctattggtcaagtcattagaggcgaagaatctgagaactccctggaggcgcttcgtgttcttgatatcatactgaggcagcattccgcagaacagtatgtagctatgcatcttgatggatgattataggtagatttgaggatcttctgcagttacgacatgacaatttttctcaatacttgcagaggctgccttttggttaagcaatcatttttctacaacaacccttcatgctttgttgacttgggtggtggtgtgatgggttgtcgtggatttcattcaagcttccgtggcacacagagtggactttccctcaatgttggtgcgtgttcgtcctgtatggctgtattggtgccatgtaactgcattgtctattctataatcttacatctttcatgatcatgatagcctttttttttgaaatccacagatgtctcaacaacaatgatcgtgaaacctggccctgttattgattttcttctttctaaccagaatgttaatgatcctagcagaattgattggcaaaaggtaaatgccatctttggatgaaacttctcgaataaccatgctagtgcattgaaatatctattttgatagaatgataacacaattttggggtgtgcatattcaatacaggccaagcgtgctctcaagggcttgaggattagaaccactcctgcaaattcagaattcaagatttttggtctcagcgagaggatctgcaaagaacaaacgtgtggacatgttttcttaccgctctgtttattgtcatgcaagctttactttacatttttaaaaaattgtaccctagtgtgttaagcttgtttcagaagaattttatttaacaaatattgcctttattgattttgttctgattccaatgccaagcttgcttgatggtgatatctgtgtgcttaattgctttctcattgacatatgcaattaggtttccgctgaggcagagaaatggtagcaacggagattgtgataccattgaaataactgtctatgactactatgcaaagaaaggaatcgatctaaagtattctggtgatttcccctgtataaatacagggaaggcaaagcgcccaacatattttccaatcgaggtttgtttcagttttgttagttacatcctgtcaaatctctgtttattaaatatatcttgcatctcatattcgatgggcaggcttggtggggctgtctcactgagtcactaggtcgtgggttcaaagcagcctctccacatttgtgggggggggaaggcttgcttcggtttatcccttccttagaccctaggtctgcccctttttttgcatctcatttttttgtattgtgtttctagctatgctcgcttgttccgcttcaaagatacaccaaagctttgtctacgctacaaaggtcatcccttgtggagaagtctagacagaagcctgaagaaaggatgaccgttctaaatgatgtgagctgttaccttgattttagcatgctgcgaccattcataactgcatgggatttatttctacgactgaattaatcaacagcttatgattaccctaaatgctaggcactgcaacgcagtaactacgattctgaccccatgttgatgtgcnnnnnnnnnnaggttgagggcatgtggtgtttcagttgctccaaaatttacccaagttgaaggaaggatccttcaagccccaaaggtnnnnnnnnnngcttgnnagagaatcctagggtgtttggatggaattctaggatttaaagatttttttcctatcctgattgttaatatctctatggatccaaacgctcccttagttgttttctctctaaatgttgtaccaagtcattatctcagtaaaatgcatctgactaaatctaatatcttgtagctgaaagccggcaatggtgatgatatcttttcacgaaatggacggtggaatttcactaatagggtcagcagctcaacatgtttcttttttaaccttctttttttctttattaggaaggatttttctgatgtttatttgccaatactttgcccctttcagaagttttatgaaacctgctctgtgaataagtgggcggtcgttaatttctctgcacgttgtgatgttcggaatcttatccgtgacctgatgaggaatgcatctgcaaagggaattgtatggtttagcttttcacatatcttctattgtagcagttttctgctgctgacgttttcttctggcagcaaatggaggaaccttttgatgtgtttgaagagagtccctctatgaggcgtgcacctgtgtcaagaagggtggatgatatgtttgggcagataaaatcaaaacttcctggagctcctaggttcctcttgtgccttctccctgagaggaaaaattgtgaaatctatggttggtttattttttgttaccactccacataaatatctcacatcttgcaataattatacctttttggtttcaaccaggtccttggaagagaaagtgcctggccgagtttggtattgtcacacagtgtctagctccattaagagtcaatgatccgtacctgcttaatttgctgatgaagatcaatgcaaaggtttgggacatgattttatctttcttttgcactgtattgttccctggttcatgattatattatcctctttcctctgtttttaactagcttggtggtctgaactcgttgctgcaagttgaagcatcttcgtcaataccacatgtgtcgcaagtacccaccatcatcttaggtatggatgtttcacatggtcatccaggacaagatagaccttcggttgcagcggtaagatgggtgtacctttacttgtacaattcttctgcccacatggaatctgagtggttttgtaaaatatgtgaaatggatgtctgttaatctgatgtaaaccatacctttcttctttgtttaggtggttagttctcgtcaatggcctcttatctctagatatagagcatcagtgcacacccaatctgccagactagaaatgatgtcctcgttgtttaagccgcggggtactgatgatgatggcctcatccggtaagagatatcttcttagtatgttgatgtatggtaagcgtgatgacttttggatgctaagtttatcttcgtatttgggacttccagggaatcactgatcgacttctacactagctctggaaagcgaaaaccagaacacataattattttcaggtctgaagctttggggttattgttagcttgtatcactaaatctttttgtagaaacaaattcttttttttgtttttgttttgagaagtatgtatggtacattctttgctgggatggattattgatactttcttagccgccgtggcgtaaatgtaaccatttgctgggttttagggatggagtcagtgaaagtcagtttacccaggtcatcaacattgagctggatcagatcatcgaggtaatacatttgtttgattcgtcctttgcacatttgcttgcttagttcaggaacgaattgcaaggtaacacatccttgtaattaatgcaggcatgtaagtttctggatgagaagtggtcacccaagttcactgtgattgttgctcaaaagaaccaccacaccaagttctttcagacggcatcaccagacaatgttcttcctggtaatatgtttatttttgtcatactaaagggagtgtggtgagatgcggtgacaataataaatgtgtttgtttgttacgcaggaactgtggtggatagtaaagtttgccatcctaagaacttcgacttctacatgtgtgcacatgctgggatgattgtgagttgatctcagaattgagcaaaaagatgctgctgtcttgttcttgtctctgaatcctagatggttcttcgtctcagggaacaacaaggccgacccactatcatgttctgcacgacgagataggtttcagtgccgacgagatgcaggagtttgttcattcgctctcttacgtgtaagtagtagtagtaaaatgctgttttggctgtgtgtgtgtgtgtgtgttttgtgcttacgctgcgtgtcctgctgtgcaggtaccagaggagcacgacagccatctcagtgggtaggccgcttgaatctaaagccatcttgtgcagcacagggacagcaggttgatttttttctagtgtctgtttttgtgtttgcagttgctccagtgtgctacgcccacctcgctgcagcccaggtgagcacgttcctgagattggaggagatgtcagacgcgtcctccagccagggaggagggcatacctcggctggcagtgctcctgtgccggagctgcctcgcctgcatgacaaagtcaggagctccatgttcttctgctagctgatgtgcgtgcgcatcaggatcgagctccatgttttgtgttagtaaggcctagttagtaaggctgtagaaagaatgtttaatgtttgcatgctaaagtccaaacaatcaaaaccactactatatctaccagagcactgatcgatcaaacaacaagagtcagcatcaatcaatcaaaaccaccactattctatctaccgatcaatctactctatacctgaagcactcactcgctcgcaaacaaccaagggatcatggatccttgagtagctccattgtccagtccctgaccttgacgaagcatttcatggacagcctctcgtacgccatctcgtacatccggaggttcatgtaggcgaagctcaagtaggtggcgatgcactcttggtgcctgtggtacagcgccgggatggtcagagcggccaccacacttgcgtagcaggcggtcgggaaatctgtaacgctgccgacgatggacacgctccacaggcagaggaaggctctgtagaagagcagcgagtccctgcccagagcgatgtcccggaaagcgtccgaggcggcgtccagagcagagcgcagcagcgccgccgcctcgtccacggcctgctgcggaacgcgcagctccgggacgggaggctgcggcctgttcaggaggcgcgccgctttggcccacaggaagagcacggtgaggagcaggagcagcacgttggaggcgagggacaccaaggtgtagccgccgtacaagagcagccaggaggagacggtgcccgccagcaggcaggcgctcacgtcggcgcggccccggcgccacagcagcacgtcccagactgctgcagcaatccgatccgattccgatccagatatattagttagtagatccaatctcgagcggccaatccgtaataataattggctgagagaaaaggtgggagagagtaccgaatcctccgacggcacatgtcccggcgccgccgccgccgccgccaacaccaacaccagcatccaccaccatcgtcgtagttaatactaagggaagggaactagagaaggaagggaggggccgatgcaacggaagaaatggatggaattcgaccaacagctagctagagcgaagcagaacccaatcccaatcaaaacccgagaacgaacctgcttttactttcccaaccagacggtggctggcttggaatcccactatccctcattccctctaccaacagggcaccggcctggcctccctccccaatcatccaaccaccacctttttttttcttccttttgctgtgttttcttacaaacttaaatctactcgaatattgattcaagatattatggagagtaattgaatatttcgatgaatcacgtcatgtatatatttcttaattacaaacttctatcactatcaaataaagtaaacataatgctatttgcaataatatatactatgtaaacatgactaaatcaaaataaatgtttaatttaatagttaccaagtttgcaagaaaacatttccttccaaagcacatatcatgatgctggcaatttagttttatcagctataataatatcattgacaacacattttgtaagtccacattctaaataaattctgtacattataactaaaacattatatcatatcagcataaaaaataaattaagtattctatgtttctataaaacaataaaccatgcattgtacggtgactgcgtctagggaaaattataccgcatgctagcaaaacgtcctccagtatttcattagacacattggtgtcaacctaatatcctatttgttgtcgttccaactaaccccctcttatagtgctacaacactatcatctatgtgtgatggttttactcgtgatacctcacttacaggtgaaaataagaccctcaaaaaggaggcgaatgagctcattcacgtcttaattaaagcctacagtagtgaggctcacttgcttaaatgcttggatagtcaaagattctaggtattagactaagtaactattctgttttgctacaaatgtattagactaatgctgaagggcaaatgccgaaggtaggtttcgaccagggcgcgcaccaagactttggtgatcattgtgaacatgcttcggacgaagctaattccgaaggtcctatgggcgacgacaaactaag

SEQ ID NO: 13 - DNA, 28526 bp, Artificial sequence
  Nucleic acid
ccaacaaactgactgatactcctttcgtccacaaatctctgtcgcccgatcttttttcatttttctattagtcttcgttgtattagcatgttcctctgtcttctcagtatgtctgtcgcttcattacttctgcgcattctttaagaatctttattgatatcgggtattatgaaaacttgtagtgattaaaagcaaatcgacgctccttggtcactgcaccaatgcctaatacgctggagatttaggaatggatggagtataatatacttagaaattaattatataaacatagttaaaaattaatatatatgaagagtcgtgtaaagataaacttttaaatatagttgagagtcaatatatttgagaagaccatcttttatcttttttatttacattaaatgatattgtacctgctctgactaaagcggtcagacggtggagacagacagaggacgactatcttttatatttttatttacattaaatgatattgtatctttattgtcttgccctaaagcggtcagtgggtggaggcagccaagagtcaccctgtcgccgttgccgtcgccgaggcaggcagagtctccctgtcgccgttgccgtcgccggcctcctcctcctctgccacttcctcccaactcccagccgcaggggccgacggcgacgggagggaagaggcggcagaggtactccgaggggcgcggagagaggctgtgccaggccacgccgtccagtgcctccgggtccgtcgccgcagcttccggccgcgtcggagaggtagcccccggagctcttcgcggaggctcgggaatggtggtgagagtttcccttcgcgtcccctcgtcttctctgctctgcctccttccgtgtactgtgaagagatgctatctcgcgcaatgaattcttgacgctaataatatggtccgctgctacggatattccgcgcgttagattggggtctttgatgtggcgaggtcattacggtgtcctagccctcatattcagcgctcagtgttggggttttcttaacactggattgaggagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttcgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttcctagagcttgcgttttggatgtttttttcgagattgatggtggtttggtcctgttgttgatcgatgtggctgatatgtacttcttaattcgtgtcacagcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggtaagccagctaacggatccatacatatatgcaacaatgtgtgagttcagcagctagttcgtgttactcaggggtgttcaatttaaaccacttgtgccaaactggccggggtggggttgggtggtggatataataatcttgaacaaacgcaggatcatgattagtctacagtgacacggacaccctcaatgttactgttaccttgtttctagtggctacgattgtaaggtttctatttgtttgtggggttggttttaaggttgctaatggcaatgggaaggctgggcgatctgcctctttggttcagaagtatgtggacatgggtttctcagaagagattgttctgaaggccatgaaggacaatggtacttaactaatagctcatctcttctcatttatttagattctatgtggtttaatataccctatatcacagtttttttttctttcaggggataatggagcagattcattagttgagctccttcttacttaccaggtattcatttgtgttcctgcttctatgttattctttgatgatgttgttgcatggtatcaaagagaatttattttacaaagtgaataagtgatgccaaatgcaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggtattttcatgctccccttggtgattgtaagcgtttactttattcttttttgagaaacctactatgcatttactttgcttcaatcatatatacaacgacaaacagcaacaacaaaatcttttattccaaacaagttgcgtagactagagttaaaaaattaatagaacaacaagtcatggttcggccacatggatagttgttttttatgaactcatattcaaggctaaatctttgggttgggtatatcccatccttatctccatttactgtctcttcccatgtcaactttagtcttcctcttcctcccttctcaattctcataactaaggcgccttagaattccactacgcacttgcgtctatggaggtctacgttggacatgtccaaaccatctcaaacgttgttggacaagtttttcttcaactggtgctactcctagcctatcacgtatatcatcattccggatttgatctcttcttgtatggccacaaattaaacacaacatacacattttcgggacacttaattgttgaacatgtcgtcttttgtagggcaacattctgcaccatacaacatagaaggtttaatcattgtcctataaatcttttcttttagcttctgtggtaccctcttgtcatatagaactccagatgcttgacgccacttcatccatccgcttcgattctatggctaacatcttcattaatatccccgtctctctatagcattgatcctaaatacctaaatgtgtccttctaggcactgcctgactttccaaactaacatctcctttctcatgtgtagtcccgaaatcacatttcatatattttgttttagttgtactgagtctaaaacctttggattctagagtctctcgccacaactctagtttcctatttactcttgcccaccacataacattccttggtatcttgtcataattttccatgtcaatgaaatgtcacacccggatttaagggataaagccgggtgtgtctcatatgtgcgccaaaaaagaacaacacatataatgataaagtgtatagagataaatgtcacaatatcataaaagtaattattacatagcggaagtcttacaaaaataaatgataaatataaaacgaactaatgtccatccatggcgccataacgttgactgggagacgccacctagttcaactcgtatgcttcattgtagggcggctcctcttcgaccacctgctattaacctgtggggggggggggggtttatatatcgcaagagtgagctcacaaaatatcatagcttaagtgattaagtgagaactcaagaagaaaaatcaatcacaataaaagatgacacaagagacacagtgattttatcccgtggttcagccaatgcctactccacgttgtggtgacctccttcggtcaaggattgcactcaatccctctcaagtgatccaacaatcaaacttgagtatcacagttttcttccttagtgatgtttcccgtttgtgaggaatctccacaagttggagcctctcacccttacaatgttgatcacaagaaaagcacaagagtaagggagggaaagaaacacacacaagactagaatcgcaacaacaatacacacacaagccaagaagagagcacacaaaaacaatgccacaagttcacaacacacttagtgctcaaatctcagtcacgatgatccaaatgcgtatttgcggagtctaggcgtcttaggatgtttagagaatgcttggtgtagtgctccatgcgcctaggggttccttttatagccccaaggcagctaggagccgttggaattccatttggaaaggtaattcttgcctttagaagcttatgaaggttcttcattccaacatgtgctagatggcggtgccagagccagcccatattagtcttagcgattaagcatgcatctagatcgacattctcttttgagaaatcaactaaatagagcttgccgtgtaatacagccttaaaagttaatgaacaatcactccttctaaagacagatacatcaatatttgtaaataaacaattatagcccatatgacataattgacttacaaacaataaattatacccgagcgattctactaaaaacacatttgaaattgaatgctcggttgtgatggctatcttgcttaagcccttaaccttgccttggtttccatctccaaaaatgatagtatcttgggaatccctgttcttgatgtaagaggtgaacattttcttctcccccgtcatgtggtttttgcatccgctatcaataatccagcttgagctcccggatgcataaacctgcaaggtaattaagcttggcttttaggtacccaagtcttgttgggtcctacaaggttagttacaacaaattttggaacccaaatgcatgtttctcccttgcatttagatcccaattttctagcaactactttggcatttttacatgacaatacaaaagaaacattacatgtttgatacatagtagttggaccagtgtatactttcctaggtgcatgagaatcactatgattacgcctaggcctacttctaccatgaacatatgtggagctagatgcaaacatagcatgagaattatgataagtaggtagagcaacatgattataacacttatcatgatgggcaacactggaaattttcttgacatgagcataaagataagcatgattcttttgaccatgtgaagaatacaagctactagccataggagccttccctttctctttgttgagactggaagtcctttggcttgttaagttcttggtttcttatcggaaaccaagtccatccttaattgaggggtgtctaccaatgatgtaggcatccctagcaaatttgagtttctcataattatctttgcaagtcttaagttgagcattaagacttgcaacatcattgtttaacttaataatagtagaattatgttcattacaagcatcaacgttaaaatttttgcatctattgcaaataacaacatgctctatggatgaactagcagaatttgcttcttttaacttagcatttaaatcatcatttaaattctttaggctagagatagattcattataagcagataattcagaagtcaatagttcattcttcttagtttctagagcaagggctttttgaacattaatgaatttatcatgttcttcataaagaatatcttcttgcttttctagaagcctatccttttcattaagagcatcaattaattcatttattttgtctactttagttttatctaatcctttaaacaaatcagtatagtccacttcatcatcagatgattcctcatcactagaagaagaatacttgggggtatctcgaatacgtacctttttctccttagccatgaggcacgtgtgtcgttcgttggagaagaggaaagacttgttgaaggtcgaggctagtccttcatcatcgaagtcggatgaagaacagtcggagtcccactctttttcaatgtgcgcctcgcctttcgccttcctgtagttcttctttctttccttcttcccatgcttctcttgtgcctggtcattatcgttatcgaggcgttgtgctataaaatgaccagacttaccgcatttgaagcaggagcattttccccttgatttgctcttgttagggtactccttgcgtccttttaaggcggtcttgaagcgcttgatgatgagtgccatctcatcctcgttgagcccggcatcctccacttgtgctagcttgctaggtagtacctccttgctgctggttgctttgagagcaacgggctgcgattcgtgtagaggaagcggtccgtttaatgcatcatccacatacctcgcttcctttaccatcatgcgcccgctaacgaattttccgatgatttcctcgggcgtcatcttggtgtacctggggttctcacgattaaggtttataacatgaggatcaataacagtaaatgacctgagcatgagtcggacgacgtcatgatccgtccatcttgtactcccgtagcttcggattttgttgacaagggtcttgagcctgttgtacgtttgtgttggctcttctccccttatcatcgcgaatctccctagctcgccttccaccagttccattttagggattatggtggcatcatttccctcgtgagaaattttgagggtgtctcatatttgtttggcattatctaggccactttgttgtattcatccctgcatagagatgctaataaaatagtagtagcttgtgtatttttgtgtatttgctcattgataaacacagggttgttagagctatcaaaatgcattcaattttcaacaatctcccagatactaggatggagggagaataaatgactacgcattttatgactccaaaaagaataatcctctccatcaaagtgtgggggttttccaagtggaatagagagcaaatgggcatttgaattgtaggggatgcgagaataatcaaaggaataattttgattaagcgttttctttttcgatgaagagtcttcgtcgtcgtcatcccttggtgaagaggaggcgtcactgtcgtagtagatgatcttctttatgtgcctcttcttctttctgtccttcttcttgtgacttgagcctgagtcagtgggcttgtcatctcttggctcgttgaagagggactccttctccttgtcgttgatcaccatccccttacccttaggatccatctctttggtcggttagtccctaaatgaagagtacgactctgctaccaattgagagcacctagaggggggtgaataggtgatcctgtaaaatcaagtactaaatagtccaaacttggttacgaaatgttagttagatcaaaaccaagatgctatagcgagaactcttcacttgattgttcctttagaatgagtattaaacttaggaacaagagcacaagtgattaagcggcctatgatggtttctttattggctcaacaacctttaggtcttggaggaggatttggaagagttgggctcctggaaaatgtaaattcttcatgtggctggtggcgcacggccgctgctggaccgctgaccggctggcaaggagaggcctgcctcatcctcaaagttgtctgttatgtgaccaggaggaagaaactatgaatcatctgctagtttcctgtgtcttctcaagggaagtctggtttaatatcctgaaaacggtcggtttgcagcacctctctcccagtctcgattcttcttcttttgatgattggtgggagaatgtaagcatgacccttattagcccctccgataagaagctgcgcaaggggctgaactcccttatcattttaggagcttgggccatttggattcatcgcaatcaatgtgtcttcaacggagagcagcctagggctagcaaggtcataaattgggtcatggctgagtctcatctctggtgtagggctggggctcgcggattatctgatatcctttttacctcggcatactatttgaggagttggtctctagtatgcttagtgcactggtgctcctgctcattttcttttgttgggtggtgtgttgagttgtatgaggttctttaacacctcaccccatctttttctacttaatataatgatacgcagctctcctgcgtattcgagaaaaaaaagaagaaaaaccaatcacaataaaagatgacacaagagacacggtgattttatcccgtgtttgaccaatgcctactccacattgtggtgacctccttcggtccaagaattgcaactcaatccctctcaaatgatccaacgatcaaacttgagtaccaccgttttcttccttagtgatgtttcccgtttgtgaggaatctctacaagttggagcctctcacccttacaatgttgatcacaagaaaagcacaagagtaagggagggaaagaaacacacacaaaacaagaatcgcagcaacaatacacacacaagccaagaagagagcacacaagaacaatgccacaagttcacaacacactcagtggtcaaatctcaatcatgatgatccaaatgcgtatttgcggagtttaggcgtcttaggatgtttagagaatgtttggtgtagtgctccatgcgcttaggggtcccttttatagccccaaggcagctatgagccattggaattccatttggaaggcaattcttgccttctgtcgggcggcacaccggacagtctggtgcaccaccggacatgaacagtccttgttcggtgcccgatctccttccttttctggcgaagccgatcgttgagcccttggtccccttgtcgcaccggacattgtccggtgcgaccaagttaccgttggcccaggccacgcgtcacccgctgattgcgctgccgaccgttggcgagggcgccgttggctcaccggacagtccggtgaattatagccgcagcgccctcggcgaattcccaagaacgacgagttcgtcgccgagccagcctgggcaccggacagtccggtgcacctcaggctggttcaagtttggctgcacttagccaaacttctccaatccaatcttagttgattaaccaaggttcctagcacttagagaaatatgttagtactcaaaaacaatttactaaggccagagtcataccttgattctttgatttgcatctcttccacacttagtacatttgaacctaagtaatttgtgttgggcatctaatcaccaaaacatttatagaaatggcccaagggcacatttccctttcacaaaaccataaagagcaatagcaaaaactacccaaatagttcaggggtaaacaaggtgaaaagataaccagactagggtgacctattgggtcccatcaaaattaaacctatgcatgaataaatgattataaagaacattattgggtaataaaagtggtaaagggcacaacttgccttcaatgagctcctgctcagtaacttctacctgctaaacaccctgatcctcggccactggctcttctactcgtcacaatacaaacaagcatggtataaggaagaaattaacatcacaccaaacatgtatacaaaatacataataataatctacacattaaaacgagatcataggaaaaagaatcactaaattcggagttacagatttcaagttatgattttccgaagtatttatgtgcttggtacaaaattgattgtgtgatcaattctaatgtgggtttcatggtaaaacagtaacactaagtgataaacaatattattacaaaattataggaactggaatggattaatttggacttaatatgaattagttttgtactaaaaatcaatttccataattatttatccaatttctctgttctctgggccgagcactaaatataaggaaacacgggggcagaatcataagatttccccagactcagagcaaccctggggtggacggcgggttgattttaagtattcctaagggctcttttgagatattgccacgcgaaggggtactgtgtgatctgagccgtccgatcaattccagacgctcttgatttaatttacccgagcgtgaaacggtacgctatggcagccattagatcgccgatcgacgacacggattcaaacaccccacgccgccaccgagacgtccgatcgggatctagcggcgcggatctgtccaatcgaaggggtacgcggcgtctaatctaagccgtccatttagcatctaacggttcacactcagtcgtctacctctcagtcgtaatagcgcgacggcggcgaattctcccgtgacggagatctctccggcgagcacccacaggcgcaccaacgccccgaagccgaaattgactaacacatacaccctacgaggacatggcgaataaactggggagtgaatttacctcggttcacggcggcaaggtctctgttcacgacacaaggcggtgcggcacccaagcgaagcaccgacgagcaattaaggttgctctgcttgtcgattcggccagcaagtgcttcggtctccctcgcagcaactcggcgcaggttcctgactcgatctccggcactggacggtgacgggaacgggaacccgtgtctccccacttctgccacccctggcttttctctccctaagctctatacgcgacggcgagggtttggcacggaagcgagaggcgcataaggttcggtgcagcagagttttatggccgggtgaggttgggcggttagcggccagctgatccacaaaccccgcgtggtggcggacatctcggccatggccgttgcggaggtgagggtgatgttttgacacggggggcccagacgcagtgagggcgcaacgcgacgtgttgagtcggtctctcggggcccacgggacagaagcttcgtgcgaccgagttacgtagactggcaagggggccccgcaagtcagtcatggattccggctgggccttgcagagaacgtgctggttgggccggctaagccggattaggcccaggcgcacagttcctttcttcctttttcttttctattttatttttcctatttctttcattttcagttttaagtttcgaatttgaattcatatgtgtggagctcatacttaggttaaatgttttcattcaaataatagtatgaatcgaatttatttatttaattaatttaatttgtatagtatttttcttcttttttctcagatcctagtattccaattagatctttaattccaaattgggccagtaatattttgctattaatattatttttattgttaccaaatgcacacacaaagaaactccgacatgatgcacgatttttttattggttattggttaattacttacttaaaataaggttgttcgcatgtgatatcaaatagaggaaaatcatatagaaaaaaattatctcctttactttgggttacaagtttgggtattacatgaaaatcatgtgtagatccttcttttgctccctatactgctctatcacttgtcttgttaagaaaactagaacatgaggcgcccgtaagggcgccctatcaggactaccgtatacaaagctgaacaatagttataaggacactaaagaaaggttcatgtatgtgaatcacatggtaacaggagtagtgatgatcgttctttattttaccaagatcgtcatccgtagccttggacttctctagaggcaacctaaaccacaacttgaaagaggcagatgaaaaatatatcattcagatcagatataagcatatctagaacaggataaactgaggaaacgaggagtgagacatgaaaggcaatacattcatatttgtcaggccttgcaaaatttgtttcacaactctagcaagatactcgatagggagggactacctctgtgcaaacatggggttgtttgcataagtagcagaccataccagtccatttaacatgtgtttagaaattaggatttcctttgcacaagaattgatcaacatgtacattggccacccgtagcaattaaaacttcacctagagacaaattgtagaatgtgatttatttatatgtagagtagagctataagtttctcgttggatactttatctgtcctaaaataggtgtatatcttgcactccgagtcgacaaattatatatttgattaagtatatgaaaaaacattaatatttatgatatcaaaacattaatatttatgatatcaaatagacatcattaggtgagtcacattgtgtgtagtaaagatattttaagatagaaatattaatactatttttctaaaaatctagtcaaagttgagaatgtctggcttctcaaaatgtgaggtctaagatgtgcacctattgtaggatggagtaactatatagggtacataaaacaaacaaacaacgtagatataccattttaaatcgaaacacaagtaaaaacaaacacctcgattgacgaatcccatttcttagtaaattagttggtggatgtgtcatgtacggtaagccgtacacaaggcgcaggcgcgctcgcgacatccggcgggcagcggagcgcgccaacagtgcagcagcgacgcagatggcaggcactcaagtttaggaaagcagcagccacctatggcaagggataaataggaaagtatcaatttggctcttatggcaaggaataagtaggaaagtagcaatttagcacttatggcaaggaataagtaggaaagtagtattttggcagggaagataattagggagcttgctccatttgtgtggccgccggccctatatatccccaagtgcgcagcactgttgagggcaataaagtatcaagtcatctcccacctctctctctcaagcctgcggttaggaggaatactcctaccggagaagacggccgaaggctacgagttacgtagccgaggcccggcgagtcaagggttcgacgctcttgacagcttggtatcccgagtccggagatccttgcctcacccagtcccgcccaccgatccactaaccacaccaccatccccgaccacgcccaccagcggttcgccatcctccatccacatggccgagcccactttagccgacgtcatcgagatgatgaaggccatgcaggccgacatcaccaggctgaaggagaagtccgactcctcctccgcaagcggcgcccagcccgagcgtcatcgcgacatcgatcgcccgccaaggttccagaaatgggactttccgcggtttgatggcaagtccgatcccctcctcttcatcaacaaatgtgagtcgtattttcgccagcagcgtacaatggtggaggagcaagtgtggatggcctcgtataatctcgagggcatcgcacagcactggtacatacaactacaggaagacgagggcaccccgtcctggagcagattcagggaactgctcaacctgcgcttcgggccagccctccgttcagcgccgttcttcgagctcaccgagtgtcgacgcaccggcacggtcgaggagtactccaaccgattccaggagctcctaccccgtgctggcaggatgacggaggagcaacgcgtccaactctacaccggcggcctgctgccaccgctcagccacgcagtgcggatactcaaccccggcaacctcaacactgctatgagcctagcgcggcaggtggagcagatggagctcgccaagatcccggcaccagccaagacagggccgcgcggcatcctgccgccgccacctccgcggccagcgactcaggcgctcccggcgccacaggcccccttggcgctgccagcaccgccactgtgcgcagcaacgggccgtggcgacggctccaggcgattatctccagaggagatggcggatcggcgccgccaaggactgtgttttaattgtaacgagaaatattctcgtggccacaaccgtttttgcaggcgcatcttcttcttggatggcgtcgaaattgaggaggccgaggcggaaacgggtgccgcggagaccgacgctccctgtttttccctccaggcagtggccggggtcgcgccaggggacaccatgcagatcgccgtcaccctcggttcagtctccctggtggcactgctcgactcgggaagcacccacaattttatatccgcagcggcggcgcaccagtcgggcatccccatccagcgccgaccccgcctcacggccatggtggccaatggcgagcggatcacctgcgcgggcgtccttcgcaatgcgccgctccacgtcaagggcgaactctacccggctgacctcttcgtcatgccattggccgggtacgacgtcgtcctcggcacacgatggcttggtgcgttggggccaatcgtgtggaacctcgccaatcgccgcatgacgttccagcgccaggggcgctccatctcctggacgggcacgcccagccccaaggagccggcgctgcgcgccaccaacggaacggacgccctactggaggcccttctcgcctcgttcgggggactcttcgcagctccgatgggtttgccccccaagcgcgcccacgaccatcgcattctcctcaagcccgatgcgtcaccggtggctgttcggccgtaccgctacccggctgcacacaaggacgagctggagcggcagtgcaacgccatgattgaccaggggatcgtgcgtcgcagcgactccccattctcatcgcccgtgctcctggtcaagaagcccgatggctcgtggcgcttctgcgtcgactacagggccctcaacgccctcaccatcaaggacgcgttccccatcccggtcgtggaggagcttctcgacgagctccacggcgccaaattcttcaccaaactggatctacggtcgggctatcatcaggtccggatgcggccagaagacgtgcacaagacagcgttccgcacacacgatggcttatatgagttcctcgtgatggcgttcgggctctgcaacgccccagccacgttccaggcactgatgaatgacgtgctccggcccttcctccgacggttcgtcctggtattctttgatgacatcctcatttacagcagcacgtgggccgatcacctccgccatctacgggccgtcctcgacgagctacaacgccatcagctgttcatcaagcgttcgaagtgttctttcggagcaacatctgtcgcctacctcggccacatcatctcggccgcaggggtggccatggacccggccaaggtggctgccattcacgactggccgccgccgcgctcggtccgggccgttcgagggttcctcggcctcaccgggtactaccgccgcttcgtccacaactacggcacggtggctgcccccttaacggccctcctcaaaaaggatggcttcgtctgggatgacgcggccatggctgcctttacggcgctcaaggcagccgtgtcgtctgctcctgtcctcgccatgccagatttcggcaagcggttcgtggtggagtgcgatgcatcctccacaggcttcggtggggtgctcgtccaggagggacacccggtcgcgttcttcagccgacctattgcaccccgccaccgcgacctcgccgcctacgagcgcgaacttatcggcctggtccaggctgtccggcactggcggccgtacctatggggacgacacttcatcgtcaagacggaccacttcagtctcaagtatctcctcgaccagaggttggccacgattccccaacaccattgggtgggcaaacttctgggattcgattttacggtcgaataccgctcgggcgccacgaacaccgtcgccgacgcactatccaggagggaccccgacgagggcacactgatggccatttcagcaccccgtttcgacttcgtcgagcgcctgcgccaggcacaggctacggatccggctctcgtcgccctctacgaagagatccgcgtcggcacgcgcgctgcaccatggtccatcatcggtgggatggtcgccatggaggggcgcctctatatcccagccacttcacccctattacgggagatcgtggccgctgtacatgatgacggccacgagggcgtccaccgcaccctgcatcgactacggcgcgactttcattttcccaacatgcgtcgtattgtgcaggaattcgtcagggcgtgcggcacatgccaaacatacaagtccgatcaccttcgtccagctggtctcctacagcccctgccgatcccaaatggggtgtggtcggacatcggcatcgacttcatcgaagctctgccccgcgtgcaaggcaagacggtcattctcactgtcgtcgaccgtttcagcaagtactgccacttcatcccactggcgcacccatacacggcggagtcggtggcgcaagcattctgcgccgaggtggtgaggctgcacggcatcccgcagtccatcgtatcggaccgagaccccgtcttcacgtcggccttctggagggagctaatggccctgactggaaccaagttgttcatgtcctccgccttccatccgcaaactgacggccagtcggaggcagccaaccgcgtcatcatgatgtaccttcgctgcttcaccggcgatcgaccccgacagtggctccggtggcttccatgggcggagtacacctacaacaccgcataccagtcctcactccgcgagacacctttccgggtggtctacggccgcgacccaccgaccatccggtcctacgagccgggtgacgcacgcgtgcccgcggtggcacaggagatggaggaacgggctgccttcctcgacgacgttcgctaccgtctccatcaggcgcaagaatcccagaaacgcgcctacgatcagcatcacaggcaggtcgtctacaatgtcggtgactgggcccttcttcgtctccgtcaacgaccatcggcatccctgccacgttccaccacgggcaagttgaagccccgctacgtcggcccctaccgtgtcaaggagatcatcaacgaggcggccgtacgccttcagcttccatccgacgccaaaatccacgacgtcttccatgtgggcatcctcaagaagttcgttggggcgccaccaacggaaccacctgcgctaccggccacactcaacggcgcagtggtgtccacaccttcccgtgtcctggcgggtcgcctggctcgcggcattcggcaagttctggtacagtggcacggtgaaccagtgtcgtctgcgacctgggaggattttgaggatttcagggcaagcttccccgctttccagctcgaggacgagctgactttcgaggccgggagagatgtcatgtacggtaagccgtacacaaggcgcaggcgcgctcgcgacatccggcgggcagcggagcgcgccaacagtgcagcagcgacgcagatggcaggcactcaagtttaggaaagcagcagccacctatggcaagggataaataggaaagtatcaatttggctcttatggcaaggaataagtaggaaagtagcaatttagcacttatggcaaggaataagtaggaaagtagtattttggcagggaagataattagggagcttgctccatttgtgtggccgccggccctatatatccccaagtgcgcagcactgttgagggcaataaagtatcaagtcatctcccacctctctctctcaagcctgcggttaggaggaatactcctaccggagaagacggccgaaggctacgagttacgtagccgaggcccggcgagtcaagggttcgacgctcttgacaggatgcgagtcaaaatcatgtcatagtagattccatctaactcaccagcacagctcaaacaaaaaatgtaatggacaataaggttattccaaaattctaattccagagtctgatcaccttcgtatgggtagtcccgatagcaacgaaactctccttggtggtggcctcatcgtctggtgtcttgcatggcagcatgctgaatgtccagccttcaaggatgcatgtcctctgggtcatccccttaaacaccatcagttgtaggaagatgaacctcagaagcatcaactctgcatcacacagagacaaagaggtaaggtttgtgccgtaaacatatgtatcttatccggaatcatagagaaaaggaaaggaagactgagggtgtgtttggattattgcatacctgaaaaaagttacgtcttgttgctgtcaacatttttaaatagccaaccgctcagtttcattttggttcaattgtacgaaggacaatcagtgtccggtgttcttgatagtcaaggcttagggcatacaagttttatcctcgtaaaactgactttaccttacattgttttgccatccttttttatctgtattatatcgttatctatgatttacttatcttgaagcgaaaatataaagattgggaaatggaggcaccttctttgaccttgagcagctcctcgaagagcggcttttggttcttcctcttgagcgtctacaggacacatcatcgaagcgctcctcgaaggcgaccccttatcttgaagcgaaaatataaagattgggaaatggaggcaccttctttgaccttgagcagttcctcgaagagcggcctctggttcttcctcttgagcgcctgcaggacacatcatcaaagcgttcctcgaagatgacctcaccggaagcgaggaacaacaacgacaagagggcgaggaggcaatagagcacgccgccacgctggggcgtggagcccgcggtggccatgcgcccgcacgtgctcaaggtcccaagcagtggggatgtcgcgcgccgccgcggcctcgggatctgcatgctcgtggggacgggcgccacgtcgtcgtctcgcgctccggctccgtccgcggaggaggctatgcgaccgtctttcgtggccagtacgagatcctatccacctcggtcgccgttgccgccatcccctgttccctagagcgttccacgaaggtgaaggaaacccttgatgcaggtgggaagagatcgcagacatcaaggtaggtggagggagcaatagcactgtagcagtagcatactactcggcttggctgttgactgttgaggaggctagaacttttcggcacggcaagttttttaatataaggattcatgagaggacatgcatgccgaaatgtttgaccagctgaacaattcgcgttcgtggtcgctttccacacgtcgggaccggtgtggggaaaatcacgcgaaacgcggagagcgttccaaaacccatcgtattttaatactttagatggctttcattgttgaccttccaggcatgaaaccaaattggttcaaagagatcctcgttattgctttcaaacgatgcttgataactctcccccatagcttcatagtatgactcatcaacttaattccttggtaattattacagctttaaatatctcccttattcttgtaaatcggtacaaataatacttctccacttgttaggcgtattgttcgatcgaaagatatggttgaacaacttggttagccatactatagcaatatcctcgaggcatcttcatacctcaattggaatatcattaggatccatcaccttaccccctttcattcttttcaaagcctccctaaccttagattcttgaatcttacgcacaaagagcctattggtgtcatcaaaagagtcatccaactgaaaggttgtgtccccattctccccattgattaatttgtcaaagtactcttaccatatatgcctgatctcgtcctccttcaccaagaggcgctctgtttcatccttaatgcacttaactcgattgaggtctcttgtcttcctgtcacgaaccctggctatcctatatctgcccttctctcctttcttcgtactcaagcattggtaaagatccttagacgcactattctttgctaccatgtatttttctatgctatccacactcctacctactcttataaagcaccagtttccttggtttccaccattgtccccaaggttttaaagtctggcaactagtcgagaagtcgatctgaacgactcgactttctctactagtcgctcgactcgttgactagtcacgagtagtaacgactagtcgcagcgattgagtcgggtgacttatttgggcctaggtataaaaatgtgagcccatccagccatctcaaccctagtaggtatgagatggaatgagatggtatgaaatgctaatagacaagttttctaccttatttatctatgatatggaatgattcatgacttgatgaggccaagacaaatggttgagatcttgtgaggcatgcaactatgttttgcaactactatgtttgcaacatccttccttacattggctaatatgtggaaacaaagacaaggatcgaaagctctctttgttagtaatgaatggtgtgagagcaaaatgaagaacactgaagtaggaaatgcacgtgttgtttgtcactctgttgcattttggcaagcagtagaagattgcatgagagcttcacaacctattctaatattcctgaggatagttgatggtgatgagagactggcaatggctatatggtgggctgccatggaccatgaaaaaagagcatcaaaaaagctttggagcacatagaatcatagaaagaataatagatgaagtaatttctattgttgataaggggtgggacagtcagatggataccaatctgtatagagcaacattgtttttggatccaaacaagttctttgaaaccaaagagaacgatagaagacttactacaatgctatattcaatatttaatgatatattgtagaagatggagatggatgaaaatatactaaacaaggtgtttttagtgttctagcatgtttttagtcttagcgtatgtagaaagttataaaaatataatcactgacttatgttcgactagtcacgactagtttgtcctagtctcgagtctctactagtcagtagttgcacgactcgactcgaaaacatcgattgtgtgggaaaccctaaccctaacaaggggttgggtgatgtattaatagactgggtagttgggcctgacccattacacaggggtgagaaggtaaatacacggggtagaccccaaaccctaaacgagtactctaatactctaacagattgtcccttttgcaaaaaaaggtccttatacttctttctaatctaactgtagcccaacttatagatcttgatttcctcccccatgtcttgcctaacaaccaaataagtgtgtgggtgtgtcacgctcgaagcgtttagatctaatttcctctccttgtctggcgtaacaactagataagataagtcttaccccttgcatgcacaaccaaccaccacgagtgcttcctcctcctcgttattggtgtcgtatattaacccctaacatcacatctctttccccatccctgcctttctcacccaaattcttccgtggaggacatgcagtaggccatcctgctggcctccaactcgccagtcgctgtctccgcgccctaccggcgccgccgcctttaccttcccccgtcggcaagcgcctcgatcttgtagcgcctcctctctttgccgccatgcgcctttgcagtcagcggggcaagaacaaccgcgggagagtaggaggcgttgccgccgctggcggtgcgtggccggacaattgagggagaggaggcgctctagattccgagtttagttttgtattggttgttggcaaaaaaattgtgagatattaaggtcgatgataatatatgtattgctcgttttcatgtaatcgttgtgcgctaatattttatcgaatatttttgtgccgtcacaacacacgtgcattatactagtcatggtacaatgttagcatttttttcttctccttaatagccctttagacttcctcattccactaccaagtatctttagcttcgcctctattctctctagttactccacacacctcttaggtggcctttcgaatgcaggttttcatcttctcccacatgttgtttgtgtcatcttccttccaagtgtcctctttgagaagcaagatgacacaaacattgaacttgcattgtattttatggtgtttggcatgtgaattcatttagctgagagttgattgaattggactacaattggacttccgtttttttacacacatttgggaggggggcgggagggcagacccacccagtggtggaggttcccacatgagtggggttggggaagggataaaccaaggcaagccttcccctcgctaatacggagatgctgattcgaacccgtgtcctcgtgactcactgggacatctctcaccactgcactaggcctgcactttatttgtttttctcatttttttttgttttcctaacttgttcatagttattgtacttcccgcttttttacacgttacaggaattgttgttttttgttcttttgaaggtaattgcaatagtagtttatgtagcttgtcaactgtattctcactgcttacatgtgctttctacacaggatttcttacatgagatgtcacggaaggacgaaaaagttgattccttagttaaaatggggtttcctgaagacgaggctgcactggctattaccagatgcggtgtgctctctgagactttgttccattctcgtctctttgcattagattcctcagcaaaataaacatcaatttccaaccttgaaagtaggatatgctctcatatagaataaatcagttgaataataacattgccatgtttaattatatgtcaagctttctattggtttactctctggattttgagaattctcttcaaatgctgtaacttggaactcgtaaattggctgcaatcttgacctgaggtaacctagaatggttgaatctttccaagcttgatttactgtttgcatcaagtttgggttaaccagttatattcaaatctgaagacaaagcatttaggcatgcccataataatctgtagaaagcaaggcgtatgataaatattatttcttacaagaataactgggtgtttaggaagttatccatccaagtagtacatgtaaacctttgtttctatgagtagcaagcattcagatgctccttcaaagcagtcaacgaacagtttattgttcgtttggggtcagaatattttaatggcatttgctgttaaataaaacctttttttgcatcttgcatgcaacttgtaccgctcagattctgcccccgtccctgttttattttaactggccatgcaccttacgccatctactgtggtctgtggttgaaaattactcagggccggatgcatctatttctgttctggtggattcaatctatgcttcacagaccgcaggagatggttactgtggcaatctgtctgactatgaggtttgtcttactgtccatacaagtctgcaataagatttagttctattataatggcctggtaatggaactgtgtctgcaatactgcaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccgccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacagatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagttcttagaatctttactgttgttttggttctagtagaatactttgtgaccgaacttgtaaatggtttgcaggttgactggtttagttctcttgccactctaatttaggctagttttttttttatttaatcttctttggtgatcttggtgcagttctgtggcactgttgggtattcaaatgcagattgatccgagaggctgccctgttgcattttctttttaaaatttgtatttgtcaatgagagatcaggtattctggttgccaaaaaaaaatctgaagttgtggagctgagaaaaggacggtgctaagctacaaccacccactgccacaccatgcgcacccccaacccaaaagatcctgcttttgctcacgcttatcaccactcccatctccatccaaaaccataaaaaaaaccagtgcgagctgaagcatccaggctctggaatcatcgtcgggatgtctgtgccagcatcagcccaggtgcctcacaccatacgatctgctgggagctgctgcttcaggtcgtcgcatcaactatctgcgcagaatctgggccgacgtgagttcccgatctcagctttgagtggccttctttttgaaagtgagcacaggttcttccaaagcccgttgtgttcgtgtagatcgcaggtggcgggggaccggaaagaagaagatcctttccacccgtggcgtgcttgagtccagaaacggcgcacatcctggtggccttatgaagagaaggtgagaacctggtgaaagacatgcggagctgtagctagacagctagctgatctagtagcagcgcacgccttgcaggaaggtcgtggagcacatcatcctgctcagagccaagcccaacatctcagacgctgaagagaaagacatgctggactacctatacacatcg

SEQ ID NO: 14 - DNA, 26772 bp, Artificial sequence
  Nucleic acid
ccaacaaactgactgatactcctttcgtccacaaatctctgtcgcccgatcttttttcatttttctattagtcttcgttgtattagcatgttcctctgtcttctcagtatgtctgtcgcttcattacttctgcgcattctttaagaatctttattgatatcgggtattatgaaaacttgtagtgattaaaagcaaatcgacgctccttggtcactgcaccaatgcctaatacgctggagatttaggaatggatggagtataatatacttagaaattaattatataaacatagttaaaaattaatatatatgaagagtcgtgtaaagataaacttttaaatatagttgagagtcaatatatttgagaagaccatcttttatcttttttatttacattaaatgatattgtacctgctctgactaaagcggtcagacggtggagacagacagaggacgactatcttttatatttttatttacattaaatgatattgtatctttattgtcttgccctaaagcggtcagtgggtggaggcagccaagagtcaccctgtcgccgttgccgtcgccgaggcaggcagagtctccctgtcgccgttgccgtcgccggcctcctcctcctctgccacttcctcccaactcccagccgcaggggccgacggcgacgggagggaagaggcggcagaggtactccgaggggcgcggagagaggctgtgccaggccacgccgtccagtgcctccgggtccgtcgccgcagcttccggccgcgtcggagaggtagcccccggagctcttcgcggaggctcgggaatggtggtgagagtttcccttcgcgtcccctcgtcttctctgctctgcctccttccgtgtactgtgaagagatgctatctcgcgcaatgaattcttgacgctaataatatggtccgctgctacggatattccgcgcgttagattggggtctttgatgtggcgaggtcattacggtgtcctagccctcatattcagcgctcagtgttggggttttcttaacactggattgaggagataagtgctattatggacgaagagggaaggggattcgacgaaatggaggcgttggcgttggcttctgtgttttggagacgcacgcgacagccaaactccaaaacggatacgagacagctcttggggctgcgtaaacaggtattagttttctgtccccgtttaccgttcccgtgcgcagacgccgtcacgcgtactcttcttgtctccgtcgccgcgctctacggcggtgctggcgtccgtgctgtactccgcgccggcagaggccgcgcgcgcgtcgcccgcggacggcgccacgccgctccacctcgccgcggccgtgcacatcctcctcgccgcgggcgcgtccgtggacgcgcgcgccttctcgggcctccgcgccggggacctcctcctcccgcgcgccaacgaggccgccgccgccgcagaccgggcgctccgcgtgctcctgaagttccccgcggcgtcactgtcgtcctcgcccaagaagtccgcctcgccgccgccggccccggaggcgaggaaggagtacccgcccgacctgacgctgccggacctcaagagcgggctgttcagcaccgacgagcaacggacgcaccttacagctgtcctagcactaattactcctaaattcatcatacaccaaagtttcaattagtaaaaggttttggtattattttctttacaagactaaaagcatccactcgtatttgccatggaaatattgccaaaatggttaccgaagaaccaatgaaaaatggcggtatccgtccacctgtaacttgtccaaggctcttccagcagagtagtgccgccatgacgattgacagagacacgagacgaaacaagcagaaggtccccgcggcgtcactgtcgtcctcgcccaagaagtccgcctcgccgccgccggccccggaggcgaggaaggagtacccgcccgacctgacgctgccggacctcaagagcgggctgttcagcaccgacgagttccgcatgtacagcttcaaggtgaagccctgctcccgcacctactcgcacaactggaccgagtgcccttcgtgcacccgggcgagaacgcgcggcgccgtgacccgcgcgtactcctacagctgcgtgccctgccccgagttccgcaagggccggcgctgccgcaagggcgacggctgcgagtacgcgcacgacgtcttcgagtgctggctccaccccggcgcagtaccggacgcgcctctgcaaggacgaggtgggctgcgcgcgccgcatctgcttcttcgcgcacaagcgcgaggagctccgcgccgtcaacccctccgcggtgttcgtcggcatgcagatgcagcccaccgtgtcgccgccgccgcccaacggcctcggcgacatgctcagcccggcggcctggccctcctcccccgcgagcaggctcaataaggccgcgctcggcggccgggacctggaccagtaccagcacatgttgttcgacaaggtgtcgtcgccgagggccagttggagacaagaagagtacgcgtgacggagatgcgacggagaaaaagggtacgcgtgacggcgtctgcacacgggaacggtaaacggggacaagaaactaatacctgtttacgcagccccaagtgctgtctcgtatccgttttggagtttggctgtcgcgtgcgtctccaaaacagagaagccaacgccaacgcctccatttcgtcgaatccccttccctcttcgtccataatggcaattatctcctcgtgttcctagagcttgcgttttggatgtttttttcgagattgatggtggtttggtcctgttgttgatcgatgtggctgatatgtacttcttaattcgtgtcacagcactgggttagcgacagtgatggcagtgataacttcgaatgggacagtgatggtaacggggagcagacagtgagcttcaacgctgctggtgctggttcatcagctctggcagcgacgaacactgatgctcctggcccatcgacacgggtaagccagctaacggatccatacatatatgcaacaatgtgtgagttcagcagctagttcgtgttactcaggggtgttcaatttaaaccacttgtgccaaactggccggggtggggttgggtggtggatataataatcttgaacaaacgcaggatcatgattagtctacagtgacacggacaccctcaatgttactgttaccttgtttctagtggctacgattgtaaggtttctatttgtttgtggggttggttttaaggttgctaatggcaatgggaaggctgggcgatctgcctctttggttcagaagtatgtggacatgggtttctcagaagagattgttctgaaggccatgaaggacaatggtacttaactaatagctcatctcttctcatttatttagattctatgtggtttaatataccctatatcacagtttttttttctttcaggggataatggagcagattcattagttgagctccttcttacttaccaggtattcatttgtgttcctgcttctatgttattctttgatgatgttgttgcatggtatcaaagagaatttattttacaaagtgaataagtgatgccaaatgcaggaactaggcaatgacctcaaagtggataatgactttgcttctagttgtgcccccaaaactgctgacgatagtgatgatgatgacacactggaaatctgggatgatgaggatgctggagggagaagcaccagggttgctaactctgttgatgattctgatgacgaggtattttcatgctccccttggtgattgtaagcgtttactttattcttttttgagaaacctactatgcatttactttgcttcaatcatatatacaacgacaaacagcaacaacaaaatcttttattccaaacaagttgcgtagactagagttaaaaaattaatagaacaacaagtcatggttcggccacatggatagttgttttttatgaactcatattcaaggctaaatctttgggttgggtatatcccatccttatctccatttactgtctcttcccatgtcaactttagtcttcctcttcctcccttctcaattctcataactaaggcgccttagaattccactacgcacttgcgtctatggaggtctacgttggacatgtccaaaccatctcaaacgttgttggacaagtttttcttcaactggtgctactcctagcctatcacgtatatcatcattccggatttgatctcttcttgtatggccacaaattaaacacaacatacacattttcgggacacttaattgttgaacatgtcgtcttttgtagggcaacattctgcaccatacaacatagaaggtttaatcattgtcctataaatcttttcttttagcttctgtggtaccctcttgtcatatagaactccagatgcttgacgccacttcatccatccgcttcgattctatggctaacatcttcattaatatccccgtctctctatagcattgatcctaaatacctaaatgtgtccttctaggcactgcctgactttccaaactaacatctcctttctcatgtgtagtcccgaaatcacatttcatatattttgttttagttgtactgagtctaaaacctttggattctagagtctctcgccacaactctagtttcctatttactcttgcccaccacataacattccttggtatcttgtcataattttccatgtcaatgaaatgtcacacccggatttaagggataaagccgggtgtgtctcatatgtgcgccaaaaaagaacaacacatataatgataaagtgtatagagataaatgtcacaatatcataaaagtaattattacatagcggaagtcttacaaaaataaatgataaatataaaacgaactaatgtccatccatggcgccataacgttgactgggagacgccacctagttcaactcgtatgcttcattgtagggcggctcctcttcgaccacctgctattaacctgtggggggggggggggtttatatatcgcaagagtgagctcacaaaatatcatagcttaagtgattaagtgagaactcaagaagaaaaatcaatcacaataaaagatgacacaagagacacagtgattttatcccgtggttcagccaatgcctactccacgttgtggtgacctccttcggtcaaggattgcactcaatccctctcaagtgatccaacaatcaaacttgagtatcacagttttcttccttagtgatgtttcccgtttgtgaggaatctccacaagttggagcctctcacccttacaatgttgatcacaagaaaagcacaagagtaagggagggaaagaaacacacacaagactagaatcgcaacaacaatacacacacaagccaagaagagagcacacaaaaacaatgccacaagttcacaacacacttagtgctcaaatctcagtcacgatgatccaaatgcgtatttgcggagtctaggcgtcttaggatgtttagagaatgcttggtgtagtgctccatgcgcctaggggttccttttatagccccaaggcagctaggagccgttggaattccatttggaaaggtaattcttgcctttagaagcttatgaaggttcttcattccaacatgtgctagatggcggtgccagagccagcccatattagtcttagcgattaagcatgcatctagatcgacattctcttttgagaaatcaactaaatagagcttgccgtgtaatacagccttaaaagttaatgaacaatcactccttctaaagacagatacatcaatatttgtaaataaacaattatagcccatatgacataattgacttacaaacaataaattatacccgagcgattctactaaaaacacatttgaaattgaatgctcggttgtgatggctatcttgcttaagcccttaaccttgccttggtttccatctccaaaaatgatagtatcttgggaatccctgttcttgatgtaagaggtgaacattttcttctcccccgtcatgtggtttttgcatccgctatcaataatccagcttgagctcccggatgcataaacctgcaaggtaattaagcttggcttttaggtacccaagtcttgttgggtcctacaaggttagttacaacaaattttggaacccaaatgcatgtttctcccttgcatttagatcccaattttctagcaactactttggcatttttacatgacaatacaaaagaaacattacatgtttgatacatagtagttggaccagtgtatactttcctaggtgcatgagaatcactatgattacgcctaggcctacttctaccatgaacatatgtggagctagatgcaaacatagcatgagaattatgataagtaggtagagcaacatgattataacacttatcatgatgggcaacactggaaattttcttgacatgagcataaagataagcatgattcttttgaccatgtgaagaatacaagctactagccataggagccttccctttctctttgttgagactggaagtcctttggcttgttaagttcttggtttcttatcggaaaccaagtccatccttaattgaggggtgtctaccaatgatgtaggcatccctagcaaatttgagtttctcataattatctttgcaagtcttaagttgagcattaagacttgcaacatcattgtttaacttaataatagtagaattatgttcattacaagcatcaacgttaaaatttttgcatctattgcaaataacaacatgctctatggatgaactagcagaatttgcttcttttaacttagcatttaaatcatcatttaaattctttaggctagagatagattcattataagcagataattcagaagtcaatagttcattcttcttagtttctagagcaagggctttttgaacattaatgaatttatcatgttcttcataaagaatatcttcttgcttttctagaagcctatccttttcattaagagcatcaattaattcatttattttgtctactttagttttatctaatcctttaaacaaatcagtatagtccacttcatcatcagatgattcctcatcactagaagaagaatacttgggggtatctcgaatacgtacctttttctccttagccatgaggcacgtgtgtcgttcgttggagaagaggaaagacttgttgaaggtcgaggctagtccttcatcatcgaagtcggatgaagaacagtcggagtcccactctttttcaatgtgcgcctcgcctttcgccttcctgtagttcttctttctttccttcttcccatgcttctcttgtgcctggtcattatcgttatcgaggcgttgtgctataaaatgaccagacttaccgcatttgaagcaggagcattttccccttgatttgctcttgttagggtactccttgcgtccttttaaggcggtcttgaagcgcttgatgatgagtgccatctcatcctcgttgagcccggcatcctccacttgtgctagcttgctaggtagtacctccttgctgctggttgctttgagagcaacgggctgcgattcgtgtagaggaagcggtccgtttaatgcatcatccacatacctcgcttcctttaccatcatgcgcccgctaacgaattttccgatgatttcctcgggcgtcatcttggtgtacctggggttctcacgattaaggtttataacatgaggatcaataacagtaaatgacctgagcatgagtcggacgacgtcatgatccgtccatcttgtactcccgtagcttcggattttgttgacaagggtcttgagcctgttgtacgtttgtgttggctcttctccccttatcatcgcgaatctccctagctcgccttccaccagttccattttagggattatggtggcatcatttccctcgtgagaaattttgagggtgtctcatatttgtttggcattatctaggccactttgttgtattcatccctgcatagagatgctaataaaatagtagtagcttgtgtatttttgtgtatttgctcattgataaacacagggttgttagagctatcaaaatgcattcaattttcaacaatctcccagatactaggatggagggagaataaatgactacgcattttatgactccaaaaagaataatcctctccatcaaagtgtgggggttttccaagtggaatagagagcaaatgggcatttgaattgtaggggatgcgagaataatcaaaggaataattttgattaagcgttttctttttcgatgaagagtcttcgtcgtcgtcatcccttggtgaagaggaggcgtcactgtcgtagtagatgatcttctttatgtgcctcttcttctttctgtccttcttcttgtgacttgagcctgagtcagtgggcttgtcatctcttggctcgttgaagagggactccttctccttgtcgttgatcaccatccccttacccttaggatccatctctttggtcggttagtccctaaatgaagagtacgactctgctaccaattgagagcacctagaggggggtgaataggtgatcctgtaaaatcaagtactaaatagtccaaacttggttacgaaatgttagttagatcaaaaccaagatgctatagcgagaactcttcacttgattgttcctttagaatgagtattaaacttaggaacaagagcacaagtgattaagcggcctatgatggtttctttattggctcaacaacctttaggtcttggaggaggatttggaagagttgggctcctggaaaatgtaaattcttcatgtggctggtggcgcacggccgctgctggaccgctgaccggctggcaaggagaggcctgcctcatcctcaaagttgtctgttatgtgaccaggaggaagaaactatgaatcatctgctagtttcctgtgtcttctcaagggaagtctggtttaatatcctgaaaacggtcggtttgcagcacctctctcccagtctcgattcttcttcttttgatgattggtgggagaatgtaagcatgacccttattagcccctccgataagaagctgcgcaaggggctgaactcccttatcattttaggagcttgggccatttggattcatcgcaatcaatgtgtcttcaacggagagcagcctagggctagcaaggtcataaattgggtcatggctgagtctcatctctggtgtagggctggggctcgcggattatctgatatcctttttacctcggcatactatttgaggagttggtctctagtatgcttagtgcactggtgctcctgctcattttcttttgttgggtggtgtgttgagttgtatgaggttctttaacacctcaccccatctttttctacttaatataatgatacgcagctctcctgcgtattcgagaaaaaaaagaagaaaaaccaatcacaataaaagatgacacaagagacacggtgattttatcccgtgtttgaccaatgcctactccacattgtggtgacctccttcggtccaagaattgcaactcaatccctctcaaatgatccaacgatcaaacttgagtaccaccgttttcttccttagtgatgtttcccgtttgtgaggaatctctacaagttggagcctctcacccttacaatgttgatcacaagaaaagcacaagagtaagggagggaaagaaacacacacaaaacaagaatcgcagcaacaatacacacacaagccaagaagagagcacacaagaacaatgccacaagttcacaacacactcagtggtcaaatctcaatcatgatgatccaaatgcgtatttgcggagtttaggcgtcttaggatgtttagagaatgtttggtgtagtgctccatgcgcttaggggtcccttttatagccccaaggcagctatgagccattggaattccatttggaaggcaattcttgccttctgtcgggcggcacaccggacagtctggtgcaccaccggacatgaacagtccttgttcggtgcccgatctccttccttttctggcgaagccgatcgttgagcccttggtccccttgtcgcaccggacattgtccggtgcgaccaagttaccgttggcccaggccacgcgtcacccgctgattgcgctgccgaccgttggcgagggcgccgttggctcaccggacagtccggtgaattatagccgcagcgccctcggcgaattcccaagaacgacgagttcgtcgccgagccagcctgggcaccggacagtccggtgcacctcaggctggttcaagtttggctgcacttagccaaacttctccaatccaatcttagttgattaaccaaggttcctagcacttagagaaatatgttagtactcaaaaacaatttactaaggccagagtcataccttgattctttgatttgcatctcttccacacttagtacatttgaacctaagtaatttgtgttgggcatctaatcaccaaaacatttatagaaatggcccaagggcacatttccctttcacaaaaccataaagagcaatagcaaaaactacccaaatagttcaggggtaaacaaggtgaaaagataaccagactagggtgacctattgggtcccatcaaaattaaacctatgcatgaataaatgattataaagaacattattgggtaataaaagtggtaaagggcacaacttgccttcaatgagctcctgctcagtaacttctacctgctaaacaccctgatcctcggccactggctcttctactcgtcacaatacaaacaagcatggtataaggaagaaattaacatcacaccaaacatgtatacaaaatacataataataatctacacattaaaacgagatcataggaaaaagaatcactaaattcggagttacagatttcaagttatgattttccgaagtatttatgtgcttggtacaaaattgattgtgtgatcaattctaatgtgggtttcatggtaaaacagtaacactaagtgataaacaatattattacaaaattataggaactggaatggattaatttggacttaatatgaattagttttgtactaaaaatcaatttccataattatttatccaatttctctgttctctgggccgagcactaaatataaggaaacacgggggcagaatcataagatttccccagactcagagcaaccctggggtggacggcgggttgattttaagtattcctaagggctcttttgagatattgccacgcgaaggggtactgtgtgatctgagccgtccgatcaattccagacgctcttgatttaatttacccgagcgtgaaacggtacgctatggcagccattagatcgccgatcgacgacacggattcaaacaccccacgccgccaccgagacgtccgatcgggatctagcggcgcggatctgtccaatcgaaggggtacgcggcgtctaatctaagccgtccatttagcatctaacggttcacactcagtcgtctacctctcagtcgtaatagcgcgacggcggcgaattctcccgtgacggagatctctccggcgagcacccacaggcgcaccaacgccccgaagccgaaattgactaacacatacaccctacgaggacatggcgaataaactggggagtgaatttacctcggttcacggcggcaaggtctctgttcacgacacaaggcggtgcggcacccaagcgaagcaccgacgagcaattaaggttgctctgcttgtcgattcggccagcaagtgcttcggtctccctcgcagcaactcggcgcaggttcctgactcgatctccggcactggacggtgacgggaacgggaacccgtgtctccccacttctgccacccctggcttttctctccctaagctctatacgcgacggcgagggtttggcacggaagcgagaggcgcataaggttcggtgcagcagagttttatggccgggtgaggttgggcggttagcggccagctgatccacaaaccccgcgtggtggcggacatctcggccatggccgttgcggaggtgagggtgatgttttgacacggggggcccagacgcagtgagggcgcaacgcgacgtgttgagtcggtctctcggggcccacgggacagaagcttcgtgcgaccgagttacgtagactggcaagggggccccgcaagtcagtcatggattccggctgggccttgcagagaacgtgctggttgggccggctaagccggattaggcccaggcgcacagttcctttcttcctttttcttttctattttatttttcctatttctttcattttcagttttaagtttcgaatttgaattcatatgtgtggagctcatacttaggttaaatgttttcattcaaataatagtatgaatcgaatttatttatttaattaatttaatttgtatagtatttttcttcttttttctcagatcctagtattccaattagatctttaattccaaattgggccagtaatattttgctattaatattatttttattgttaccaaatgcacacacaaagaaactccgacatgatgcacgatttttttattggttattggttaattacttacttaaaataaggttgttcgcatgtgatatcaaatagaggaaaatcatatagaaaaaaattatctcctttactttgggttacaagtttgggtattacatgaaaatcatgtgtagatccttcttttgctccctatactgctctatcacttgtcttgttaagaaaactagaacatgaggcgcccgtaagggcgccctatcaggactaccgtatacaaagctgaacaatagttataaggacactaaagaaaggttcatgtatgtgaatcacatggtaacaggagtagtgatgatcgttctttattttaccaagatcgtcatccgtagccttggacttctctagaggcaacctaaaccacaacttgaaagaggcagatgaaaaatatatcattcagatcagatataagcatatctagaacaggataaactgaggaaacgaggagtgagacatgaaaggcaatacattcatatttgtcaggccttgcaaaatttgtttcacaactctagcaagatactcgatagggagggactacctctgtgcaaacatggggttgtttgcataagtagcagaccataccagtccatttaacatgtgtttagaaattaggatttcctttgcacaagaattgatcaacatgtacattggccacccgtagcaattaaaacttcacctagagacaaattgtagaatgtgatttatttatatgtagagtagagctataagtttctcgttggatactttatctgtcctaaaataggtgtatatcttgcactccgagtcgacaaattatatatttgattaagtatatgaaaaaacattaatatttatgatatcaaaacattaatatttatgatatcaaatagacatcattaggtgagtcacattgtgtgtagtaaagatattttaagatagaaatattaatactatttttctaaaaatctagtcaaagttgagaatgtctggcttctcaaaatgtgaggtctaagatgtgcacctattgtaggatggagtaactatatagggtacataaaacaaacaaacaacgtagatataccattttaaatcgaaacacaagtaaaaacaaacacctcgattgacgaatcccatttcttagtaaattagttggtggatgtgtcatgtacggtaagccgtacacaaggcgcaggcgcgctcgcgacatccggcgggcagcggagcgcgccaacagtgcagcagcgacgcagatggcaggcactcaagtttaggaaagcagcagccacctatggcaagggataaataggaaagtatcaatttggctcttatggcaaggaataagtaggaaagtagcaatttagcacttatggcaaggaataagtaggaaagtagtattttggcagggaagataattagggagcttgctccatttgtgtggccgccggccctatatatccccaagtgcgcagcactgttgagggcaataaagtatcaagtcatctcccacctctctctctcaagcctgcggttaggaggaatactcctaccggagaagacggccgaaggctacgagttacgtagccgaggcccggcgagtcaagggttcgacgctcttgacagcttggtatcccgagtccggagatccttgcctcacccagtcccgcccaccgatccactaaccacaccaccatccccgaccacgcccaccagcggttcgccatcctccatccacatggccgagcccactttagccgacgtcatcgagatgatgaaggccatgcaggccgacatcaccaggctgaaggagaagtccgactcctcctccgcaagcggcgcccagcccgagcgtcatcgcgacatcgatcgcccgccaaggttccagaaatgggactttccgcggtttgatggcaagtccgatcccctcctcttcatcaacaaatgtgagtcgtattttcgccagcagcgtacaatggtggaggagcaagtgtggatggcctcgtataatctcgagggcatcgcacagcactggtacatacaactacaggaagacgagggcaccccgtcctggagcagattcagggaactgctcaacctgcgcttcgggccagccctccgttcagcgccgttcttcgagctcaccgagtgtcgacgcaccggcacggtcgaggagtactccaaccgattccaggagctcctaccccgtgctggcaggatgacggaggagcaacgcgtccaactctacaccggcggcctgctgccaccgctcagccacgcagtgcggatactcaaccccggcaacctcaacactgctatgagcctagcgcggcaggtggagcagatggagctcgccaagatcccggcaccagccaagacagggccgcgcggcatcctgccgccgccacctccgcggccagcgactcaggcgctcccggcgccacaggcccccttggcgctgccagcaccgccactgtgcgcagcaacgggccgtggcgacggctccaggcgattatctccagaggagatggcggatcggcgccgccaaggactgtgttttaattgtaacgagaaatattctcgtggccacaaccgtttttgcaggcgcatcttcttcttggatggcgtcgaaattgaggaggccgaggcggaaacgggtgccgcggagaccgacgctccctgtttttccctccaggcagtggccggggtcgcgccaggggacaccatgcagatcgccgtcaccctcggttcagtctccctggtggcactgctcgactcgggaagcacccacaattttatatccgcagcggcggcgcaccagtcgggcatccccatccagcgccgaccccgcctcacggccatggtggccaatggcgagcggatcacctgcgcgggcgtccttcgcaatgcgccgctccacgtcaagggcgaactctacccggctgacctcttcgtcatgccattggccgggtacgacgtcgtcctcggcacacgatggcttggtgcgttggggccaatcgtgtggaacctcgccaatcgccgcatgacgttccagcgccaggggcgctccatctcctggacgggcacgcccagccccaaggagccggcgctgcgcgccaccaacggaacggacgccctactggaggcccttctcgcctcgttcgggggactcttcgcagctccgatgggtttgccccccaagcgcgcccacgaccatcgcattctcctcaagcccgatgcgtcaccggtggctgttcggccgtaccgctacccggctgcacacaaggacgagctggagcggcagtgcaacgccatgattgaccaggggatcgtgcgtcgcagcgactccccattctcatcgcccgtgctcctggtcaagaagcccgatggctcgtggcgcttctgcgtcgactacagggccctcaacgccctcaccatcaaggacgcgttccccatcccggtcgtggaggagcttctcgacgagctccacggcgccaaattcttcaccaaactggatctacggtcgggctatcatcaggtccggatgcggccagaagacgtgcacaagacagcgttccgcacacacgatggcttatatgagttcctcgtgatggcgttcgggctctgcaacgccccagccacgttccaggcactgatgaatgacgtgctccggcccttcctccgacggttcgtcctggtattctttgatgacatcctcatttacagcagcacgtgggccgatcacctccgccatctacgggccgtcctcgacgagctacaacgccatcagctgttcatcaagcgttcgaagtgttctttcggagcaacatctgtcgcctacctcggccacatcatctcggccgcaggggtggccatggacccggccaaggtggctgccattcacgactggccgccgccgcgctcggtccgggccgttcgagggttcctcggcctcaccgggtactaccgccgcttcgtccacaactacggcacggtggctgcccccttaacggccctcctcaaaaaggatggcttcgtctgggatgacgcggccatggctgcctttacggcgctcaaggcagccgtgtcgtctgctcctgtcctcgccatgccagatttcggcaagcggttcgtggtggagtgcgatgcatcctccacaggcttcggtggggtgctcgtccaggagggacacccggtcgcgttcttcagccgacctattgcaccccgccaccgcgacctcgccgcctacgagcgcgaacttatcggcctggtccaggctgtccggcactggcggccgtacctatggggacgacacttcatcgtcaagacggaccacttcagtctcaagtatctcctcgaccagaggttggccacgattccccaacaccattgggtgggcaaacttctgggattcgattttacggtcgaataccgctcgggcgccacgaacaccgtcgccgacgcactatccaggagggaccccgacgagggcacactgatggccatttcagcaccccgtttcgacttcgtcgagcgcctgcgccaggcacaggctacggatccggctctcgtcgccctctacgaagagatccgcgtcggcacgcgcgctgcaccatggtccatcatcggtgggatggtcgccatggaggggcgcctctatatcccagccacttcacccctattacgggagatcgtggccgctgtacatgatgacggccacgagggcgtccaccgcaccctgcatcgactacggcgcgactttcattttcccaacatgcgtcgtattgtgcaggaattcgtcagggcgtgcggcacatgccaaacatacaagtccgatcaccttcgtccagctggtctcctacagcccctgccgatcccaaatggggtgtggtcggacatcggcatcgacttcatcgaagctctgccccgcgtgcaaggcaagacggtcattctcactgtcgtcgaccgtttcagcaagtactgccacttcatcccactggcgcacccatacacggcggagtcggtggcgcaagcattctgcgccgaggtggtgaggctgcacggcatcccgcagtccatcgtatcggaccgagaccccgtcttcacgtcggccttctggagggagctaatggccctgactggaaccaagttgttcatgtcctccgccttccatccgcaaactgacggccagtcggaggcagccaaccgcgtcatcatgatgtaccttcgctgcttcaccggcgatcgaccccgacagtggctccggtggcttccatgggcggagtacacctacaacaccgcataccagtcctcactccgcgagacacctttccgggtggtctacggccgcgacccaccgaccatccggtcctacgagccgggtgacgcacgcgtgcccgcggtggcacaggagatggaggaacgggctgccttcctcgacgacgttcgctaccgtctccatcaggcgcaagaatcccagaaacgcgcctacgatcagcatcacaggcaggtcgtctacaatgtcggtgactgggcccttcttcgtctccgtcaacgaccatcggcatccctgccacgttccaccacgggcaagttgaagccccgctacgtcggcccctaccgtgtcaaggagatcatcaacgaggcggccgtacgccttcagcttccatccgacgccaaaatccacgacgtcttccatgtgggcatcctcaagaagttcgttggggcgccaccaacggaaccacctgcgctaccggccacactcaacggcgcagtggtgtccacaccttcccgtgtcctggcgggtcgcctggctcgcggcattcggcaagttctggtacagtggcacggtgaaccagtgtcgtctgcgacctgggaggattttgaggatttcagggcaagcttccccgctttccagctcgaggacgagctgactttcgaggccgggagagatgtcatgtacggtaagccgtacacaaggcgcaggcgcgctcgcgacatccggcgggcagcggagcgcgccaacagtgcagcagcgacgcagatggcaggcactcaagtttaggaaagcagcagccacctatggcaagggataaataggaaagtatcaatttggctcttatggcaaggaataagtaggaaagtagcaatttagcacttatggcaaggaataagtaggaaagtagtattttggcagggaagataattagggagcttgctccatttgtgtggccgccggccctatatatccccaagtgcgcagcactgttgagggcaataaagtatcaagtcatctcccacctctctctctcaagcctgcggttaggaggaatactcctaccggagaagacggccgaaggctacgagttacgtagccgaggcccggcgagtcaagggttcgacgctcttgacaggatgcgagtcaaaatcatgtcatagtagattccatctaactcaccagcacagctcaaacaaaaaatgtaatggacaataaggttattccaaaattctaattccagagtctgatcaccttcgtatgggtagtcccgatagcaacgaaactctccttggtggtggcctcatcgtctggtgtcttgcatggcagcatgctgaatgtccagccttcaaggatgcatgtcctctgggtcatccccttaaacaccatcagttgtaggaagatgaacctcagaagcatcaactctgcatcacacagagacaaagaggtaaggtttgtgccgtaaacatatgtatcttatccggaatcatagagaaaaggaaaggaagactgagggtgtgtttggattattgcatacctgaaaaaagttacgtcttgttgctgtcaacatttttaaatagccaaccgctcagtttcattttggttcaattgtacgaaggacaatcagtgtccggtgttcttgatagtcaaggcttagggcatacaagttttatcctcgtaaaactgactttaccttacattgttttgccatccttttttatctgtattatatcgttatctatgatttacttatcttgaagcgaaaatataaagattgggaaatggaggcaccttctttgaccttgagcagctcctcgaagagcggcttttggttcttcctcttgagcgtctacaggacacatcatcgaagcgctcctcgaaggcgaccccttatcttgaagcgaaaatataaagattgggaaatggaggcaccttctttgaccttgagcagttcctcgaagagcggcctctggttcttcctcttgagcgcctgcaggacacatcatcaaagcgttcctcgaagatgacctcaccggaagcgaggaacaacaacgacaagagggcgaggaggcaatagagcacgccgccacgctggggcgtggagcccgcggtggccatgcgcccgcacgtgctcaaggtcccaagcagtggggatgtcgcgcgccgccgcggcctcgggatctgcatgctcgtggggacgggcgccacgtcgtcgtctcgcgctccggctccgtccgcggaggaggctatgcgaccgtctttcgtggccagtacgagatcctatccacctcggtcgccgttgccgccatcccctgttccctagagcgttccacgaaggtgaaggaaacccttgatgcaggtgggaagagatcgcagacatcaaggtaggtggagggagcaatagcactgtagcagtagcatactactcggcttggctgttgactgttgaggaggctagaacttttcggcacggcaagttttttaatataaggattcatgagaggacatgcatgccgaaatgtttgaccagctgaacaattcgcgttcgtggtcgctttccacacgtcgggaccggtgtggggaaaatcacgcgaaacgcggagagcgttccaaaacccatcgtattttaatactttagatggctttcattgttgaccttccaggcatgaaaccaaattggttcaaagagatcctcgttattgctttcaaacgatgcttgataactctcccccatagcttcatagtatgactcatcaacttaattccttggtaattattacagctttaaatatctcccttattcttgtaaatcggtacaaataatacttctccacttgttaggcgtattgttcgatcgaaagatatggttgaacaacttggttagccatactatagcaatatcctcgaggcatcttcatacctcaattggaatatcattaggatccatcaccttaccccctttcattcttttcaaagcctccctaaccttagattcttgaatcttacgcacaaagagcctattggtgtcatcaaaagagtcatccaactgaaaggttgtgtccccattctccccattgattaatttgtcaaagtactcttaccatatatgcctgatctcgtcctccttcaccaagaggcgctctgtttcatccttaatgcacttaactcgattgaggtctcttgtcttcctgtcacgaaccctggctatcctatatctgcccttctctcctttcttcgtactcaagcattggtaaagatccttagacgcactattctttgctaccatgtatttttctatgctatccacactcctacctactcttataaagcaccagtttccttggtttccaccattgtccccaaggttttaaagtctggcaactagtcgagaagtcgatctgaacgactcgactttctctactagtcgctcgactcgttgactagtcacgagtagtaacgactagtcgcagcgattgagtcgggtgacttatttgggcctaggtataaaaatgtgagcccatccagccatctcaaccctagtaggtatgagatggaatgagatggtatgaaatgctaatagacaagttttctaccttatttatctatgatatggaatgattcatgacttgatgaggccaagacaaatggttgagatcttgtgaggcatgcaactatgttttgcaactactatgtttgcaacatccttccttacattggctaatatgtggaaacaaagacaaggatcgaaagctctctttgttagtaatgaatggtgtgagagcaaaatgaagaacactgaagtaggaaatgcacgtgttgtttgtcactctgttgcattttggcaagcagtagaagattgcatgagagcttcacaacctattctaatattcctgaggatagttgatggtgatgagagactggcaatggctatatggtgggctgccatggaccatgaaaaaagagcatcaaaaaagctttggagcacatagaatcatagaaagaataatagatgaagtaatttctattgttgataaggggtgggacagtcagatggataccaatctgtatagagcaacattgtttttggatccaaacaagttctttgaaaccaaagagaacgatagaagacttactacaatgctatattcaatatttaatgatatattgtagaagatggagatggatgaaaatatactaaacaaggtgtttttagtgttctagcatgtttttagtcttagcgtatgtagaaagttataaaaatataatcactgacttatgttcgactagtcacgactagtttgtcctagtctcgagtctctactagtcagtagttgcacgactcgactcgaaaacatcgattgtgtgggaaaccctaaccctaacaaggggttgggtgatgtattaatagactgggtagttgggcctgacccattacacaggggtgagaaggtaaatacacggggtagaccccaaaccctaaacgagtactctaatactctaacagattgtcccttttgcaaaaaaaggtccttatacttctttctaatctaactgtagcccaacttatagatcttgatttcctcccccatgtcttgcctaacaaccaaataagtgtgtgggtgtgtcacgctcgaagcgtttagatctaatttcctctccttgtctggcgtaacaactagataagataagtcttaccccttgcatgcacaaccaaccaccacgagtgcttcctcctcctcgttattggtgtcgtatattaacccctaacatcacatctctttccccatccctgcctttctcacccaaattcttccgtggaggacatgcagtaggccatcctgctggcctccaactcgccagtcgctgtctccgcgccctaccggcgccgccgcctttaccttcccccgtcggcaagcgcctcgatcttgtagcgcctcctctctttgccgccatgcgcctttgcagtcagcggggcaagaacaaccgcgggagagtaggaggcgttgccgccgctggcggtgcgtggccggacaattgagggagaggaggcgctctagattccgagtttagttttgtattggttgttggcaaaaaaattgtgagatattaaggtcgatgataatatatgtattgctcgttttcatgtaatcgttgtgcgctaatattttatcgaatatttttgtgccgtcacaacacacgtgcattatactagtcatggtacaatgttagcatttttttcttctccttaatagccctttagacttcctcattccactaccaagtatctttagcttcgcctctattctctctagttactccacacacctcttaggtggcctttcgaatgcaggttttcatcttctcccacatgttgtttgtgtcatcttccttccaagtgtcctctttgagaagcaagatgacacaaacattgaacttgcattgtattttatggtgtttggcatgtgaattcatttagctgagagttgattgaattggactacaattggacttccgtttttttacacacatttgggaggggggcgggagggcagacccacccagtggtggaggttcccacatgagtggggttggggaagggataaaccaaggcaagccttcccctcgctaatacggagatgctgattcgaacccgtgtcctcgtgactcactgggacatctctcaccactgcactaggcctgcactttatttgtttttctcatttttttttgttttcctaacttgttcatagttattgtacttcccgcttttttacacgttacaggaattgttgttttttgttcttttgaaggtaattgcaatagtagtttatgtagcttgtcaactgtattctcactgcttacatgtgctttctacacaggatttcttacatgagatgtcacggaaggacgaaaaagttgattccttagttaaaatggggtttcctgaagacgaggctgcactggctattaccagatgcggtgtgctctctgagactttgttccattctcgtctctttgcattagattcctcagcaaaataaacatcaatttccaaccttgaaagtaggatatgctctcatatagaataaatcagttgaataataacattgccatgtttaattatatgtcaagctttctattggtttactctctggattttgagaattctcttcaaatgctgtaacttggaactcgtaaattggctgcaatcttgacctgaggtaacctagaatggttgaatctttccaagcttgatttactgtttgcatcaagtttgggttaaccagttatattcaaatctgaagacaaagcatttaggcatgcccataataatctgtagaaagcaaggcgtatgataaatattatttcttacaagaataactgggtgtttaggaagttatccatccaagtagtacatgtaaacctttgtttctatgagtagcaagcattcagatgctccttcaaagcagtcaacgaacagtttattgttcgtttggggtcagaatattttaatggcatttgctgttaaataaaacctttttttgcatcttgcatgcaacttgtaccgctcagattctgcccccgtccctgttttattttaactggccatgcaccttacgccatctactgtggtctgtggttgaaaattactcagggccggatgcatctatttctgttctggtggattcaatctatgcttcacagaccgcaggagatggttactgtggcaatctgtctgactatgaggtttgtcttactgtccatacaagtctgcaataagatttagttctattataatggcctggtaatggaactgtgtctgcaatactgcaggataattcctatggagggagaagcacagggaacaagaaaaagagaaaaagatatggaggccaagcacagggaagtagaggcccattagatggtagctgtgatgaacccatgcctctcccacatccaatggttggatttaacttgccagaccagtggtcaagacgagtggacagatcgttgcctgcacaagctattggtccgccgtacttctactacgagaacgttgctcttgctccaaaaggtgtctggactaccatatcaagattcttgtatgatattcaaccagagtttgtggactcaaagtacttctgtgctgctgccaggaaaaggggttacatacacaacctgccacttgagaacaggtcacctctcctccccatacccccaaagacgatatcggaagcatttcctcggaccaagaggtggtggccttcatgggacccaagacgacagttcaattgcctccagacttgcgtgtctagtgcaaaattgttagagaggattcgcgtagccctcacaaacagttcagacccacctcctccaagagttcagaagtatgtgttggaggagtgtaggaaatggaacctggcatgggttggcttaaacaaggttgctcctctagagcctgacgagatggagtttctactcggctttccgaaggatcacacgagaggtatcagcaggacagagaggtatcgatctctaggaaattcatttcaggtcgatactgttgcttaccatctctcagttctgaaggatctgttcccacaaggcatgaatgtgctgtctttattctctggtattggaggagcagaggtggctctccacaggcttggcatacagatgaacacggttatttcagtggagaagtccgaggtcaaccggacgattctgaagagttggtgggatcagacgcagacgggtactctgattgagatcactgatgtgcagacactgtcatctgagaggatcgaggcgtatattagaagaattgggggcttcgatcttgtgattggtggaagtccctgtaacaacctcactgggagcaaccgtcaccacagagatggtttggagggcgagcattctgcattgttccatcattattttaggatcttacacgccgtcaagtccatcatggagcgtttgtagttcttagaatctttactgttgttttggttctagtagaatactttgtgaccgaacttgtaaatggtttgcaggttgactggtttagttctcttgccactctaatttaggctagttttttttttatttaatcttctttggtgatcttggtgcagttctgtggcactgttgggtattcaaatgcagattgatccgagaggctgccctgttgcattttctttttaaaatttgtatttgtcaatgagagatcaggtattctggttgccaaaaaaaaatctgaagttgtggagctgagaaaaggacggtgctaagctacaaccacccactgccacaccatgcgcacccccaacccaaaagatcctgcttttgctcacgcttatcaccactcccatctccatccaaaaccataaaaaaaaccagtgcgagctgaagcatccaggctctggaatcatcgtcgggatgtctgtgccagcatcagcccaggtgcctcacaccatacgatctgctgggagctgctgcttcaggtcgtcgcatcaactatctgcgcagaatctgggccgacgtgagttcccgatctcagctttgagtggccttctttttgaaagtgagcacaggttcttccaaagcccgttgtgttcgtgtagatcgcaggtggcgggggaccggaaagaagaagatcctttccacccgtggcgtgcttgagtccagaaacggcgcacatcctggtggccttatgaagagaaggtgagaacctggtgaaagacatgcggagctgtagctagacagctagctgatctagtagcagcgcacgccttgcaggaaggtcgtggagcacatcatcctgctcagagccaagcccaacatctcagacgctgaagagaaagacatgctggactacctatacacatcg